(19) United States
(12) Patent Application Publication
(10) Pub. No.: US 2003/0077591 A1
(43) Pub. Date: Apr. 24, 2003

(54) Nucleic acid for regulating the ABCA7 gene, molecules modulating its activity and therapeutic applications

(76) Inventors: Patrice Denefle, Saint Maur (FR); Marie-Francoise Rosier-Montus, Antony (FR); Catherine Prades, Thiais (FR); Isabelle Arnould-Reguigne, Sur Marne (FR); Jose Osorio Y Fortea, Evry (FR); Nicolas Duverger, Paris (FR); Giovanna Chimini, Marseille (FR)

Correspondence Address: Finnegan, Henderson, Farabow,, Garrett & Dunner, L.L.P., 1300 I Street, NW, Washington, DC, 20005-3315, US

(21) Appl. No.: 09/983,446

(22) Filed: Oct. 24, 2001

Related U.S. Application Data
(60) Provisional application No. 60/253,141, filed on Nov. 28, 2000.

(51) Int. Cl. C12Q 1/68; A61K 48/00; C07H 21/04

(52) U.S. Cl. 435/6; 514/44; 536/23.2

(57) ABSTRACT

The present invention relates to nucleic acid sequences that regulate the transcription of the ABCA7 gene, which may be involved in the metabolism of lipids in hematopoietic tissues, as well as in cell signaling mechanisms linked to the immune reaction and to inflammation. The invention also relates to polypeptides and polynucleotides that may be involved in diseases associated with the genetic locus q13 of chromosome 19.

This application is the national stage of international application No. FR 00/13649, filed Oct. 24, 2000, which is incorporated by reference herein. This application claims the benefit of U.S. Provisional Application No. 60/253,151, filed Nov. 28, 2000, which is incorporated herein in its entirety for any purpose

The present invention relates to a nucleic acid capable of regulating the transcription of the ABCA7 gene, a gene that, under appropriate conditions, is involved in the metabolism of lipids in the hematopoietic tissues, as well as in cell signaling mechanisms linked to the immune reaction and to inflammation.

The present invention also describes polypeptides and polynucleotides, the impairment of whose sequence or expression is potentially implicated in diseases associated with the genetic locus q13 of chromosome 19.

The present invention also relates to nucleotide constructs comprising a polynucleotide encoding a polypeptide or producing a nucleic acid of interest, placed under the control of a nucleic acid for regulating the human or murine ABCA7 gene.

The invention also relates to recombinant vectors, transformed host cells, and nonhuman transgenic mammals comprising a nucleic acid for regulating the transcript ion of the human and mouse ABCA7 gene or an abovementioned nucleotide construct, as well as methods for screening molecules or substances capable of modulating the activity of the nucleic acid for regulating the ABCA7 gene.

The invention in addition relates to methods which make it possible to detect an impairment of the transcription of the ABCA7 gene and thus to diagnose a possible dysfunction in lipid metabolism at the level of hematopoietic tissues and in the cell signaling mechanisms of immunity.

Its subject is also substances or molecules modulating the activity of the nucleic acid for regulating the transcription of the ABCA7 gene as well as pharmaceutical compositions containing such substances or such molecules.

The ABC (ATP-Binding Cassette) transport proteins constitute a superfamily which is extremely well conserved during evolution, from bacteria to humans. These proteins are involved in membrane transport of various substrates, for example ions, amino acids, peptides, sugars, vitamins or steroid hormones (Higgins et al., Annu Rev. Cell Biol, 8, (1992) 67-113).

The characterization of the complete amino acid sequence of some ABC transporters has made it possible to define a common general structure comprising in particular two nucleotide binding folds (NBF) with Walker A and B type units as well as two transmembrane domains, each of the transmembrane domains consisting of six helices (Klein et al., BBA, 1461 (1999), 237-262). The specificity of the ABC transporters for the various transported molecules appears to be determined by the structure of the transmembrane domains, whereas the energy necessary for the transport activity is provided by the degradation of ATP at the level of the NBF fold (Dean et al., Curr. Opin. Genet. Dev, 5 (1995) 779-785).

Several ABC transport proteins have been identified in humans and a number of them have been associated with various diseases.

For example, cystic fibrosis is caused by mutations in the CFTR (cystic fibrosis transmembrane conductance regulator) gene, also designated ABCC7,

Moreover, some multidrug resistance phenotypes in tumor cells have been associated with mutations in the genes encoding MDR (multidrug resistance) proteins, also designated ABCB, which also have an ABC transporter structure.

Other ABC transporters have been associated with neuronal and tumor conditions (U.S. Pat. No. 5,858,719) or are potentially involved in diseases caused by impairment of the homeostasis of metals, in particular the ABC-3 protein.

Likewise, another ABC transporter, designated PFIC2 or ABCB11, appears to be involved in a form of progressive familial intrahepatic cholestasia, this protein being potentially responsible, in humans, for the export of bile salts.

A subfamily A of ABC transporters, designated ABCA, has also been identified. It is characterized by the presence of a highly hydrophobic segment (HH1: highly hydrophobic) between the two transmembrane domains, bound to the two NBF units (Broccardo et al., BBA 1461 (1999) 395-404). Four members of this subfamily have so far been characterized. They are the transporters ABCA1 and ABCA2, both located on chromosome 9, at the loci 9q22-9q31 and 9q34, respectively, as well as the transporter ABCA3 located on chromosome 16p13.3, and finally the transporter ABCA4 or ABCR located on chromosome 1p22 (Broccardo et al., 1999). The members of this subfamily are also highly conserved during evolution of multicellular eukaryotes. By way of examples, the transporters ABCA1 and ABCA4, which are the best known, exhibit 95% and 88% identity, respectively, with their murine orthologs. Members of this subfamily are in addition closely related since, for example, the transporters ABCA1 and ABCA4 exhibit a protein sequence identity of 50.9%, as well as a very similar genomic organization (Allikmets et al., Nat. Genet. (1997) 15, 236-246; Broccardo et al., Biochim. Biophys. Acta (1999) 1461, 395-404; Luciani et al., Genomics (1994) 21(1), 150-9; Remaley et al., Proc. Natl. Acad. Sci. USA (1999) 96(22), 12685-90).

Moreover, members of the subfamily A appear to exhibit a similar functional specialization at the level of the transport of membrane lipids and phospholipids. It has indeed been shown that the loss of the function of these transporters affects the renewal of the phospholipids of the cell membrane bilayer. In the case of ABCA4, there is observed, in a first instance, a normal renewal of phosphatidyl-ethanolamine (PE) in the rod cell of the membrane portion, which leads, via a succession of events, to a total loss of visual acuity (Weng et al., Cell (1999) 98(1), 13-23). In the case of ABCA1, an abnormal distribution of the membrane phospholipids in plasma membrane layers is observed, which results more precisely in the presence of a larger quantity of phosphatidylserine in the outer layer, and in a disruption of the Ca2⁺ concentration.

The transporters ABCA1 and ABCA4 have been particularly studied. The ABCA1 gene indeed appears to be involved in pathologies linked to a cholesterol metabolism dysfunction which induces diseases such as atherosclerosis, or familial HDL deficiencies (FHD) such a Tangier disease (FR 99/7684000; Rust et al., Nat. Genet., 22 (1999) 352-355; Brooks-Wilson et al., Nat. Genet., 22 (1999) 336-345; Bodzioch et al., Nat. Genet. 22 (1999) 347-351; Orso et al., Nat. Genet, 24 (2000) 192-196). Tangier disease would appear to be linked to a cellular defect in the translocation of cellular cholesterol which causes degradation of the HDLs, and thereby a disruption in lipoprotein metabolism. Thus, it would appear that the HDL particles which do not incorporate cholesterol from peripheral cells are not metabolized correctly but are on the contrary rapidly eliminated from the body. The plasma HDL concentration in these patients is therefore extremely reduced and the HDLs no longer ensure the return of cholesterol to the liver. This cholesterol accumulates in these peripheral cells and causes characteristic clinical manifestations such as the formation of orange-colored tonsils. Furthermore, other lipoprotein disruptions such as overproduction of triglycerides as well as increased intracellular synthesis and catabolism of phospholipids are observed.

The ABCA4 transporter has moreover been associated with degenerative and inflammatory eye diseases such as Stargardt's recessive disease (Allikmets et al., 1997) and degeneration of the macular region of the retina linked to age (AMD) (Allikmets et al., Nat. Genet. 15 (1997) 236-246; Allikmets et al., Science, 277 (1997) 1805-1807; Cremers et al., Hum. Mol. Genet. (1998), 7(3), 355-62; Martinez-Mir et al., Nat. Genet. 18 (1998) 11-12; Weng et al., Cell (1999) 98(1), 13-23).

In humans, a cDNA comprising the entire open reading frame of a new member of the A subfamily of ABC (ATP-Binding Cassette) transporters was recently cloned from human macrophage RNA, and designated ABCA7 (Kaminski et al., BBR, 273(2000), 532-538).

The characterization of the complete amino acid sequence of ABCA7 indicates that the protein product has the general structure characteristic of ABCA transporters in that it comprises the symmetrical structure comprising the two transmembrane domains and two NBF units. In addition to these characteristic units the ABCA7 protein has other units which were recently identified as being characteristic of the ABCA transporters, namely the HH1 region and the hot spot region (Broccardo et al., Biochim. Biophys. Acta (1999) 1461, 395-404).

Like the other members of the A subfamily of ABC transporters, the sequence of the ABCA7 protein is highly conserved in mice and in humans, with an inter-species identity of 79%. The ABCA7 protein exhibits furthermore an intron-exon organization characteristic of the members of the ABCA subfamily, as well as a high sequence homology in particular with the human transporters ABCA1 and ABCA4, of 54% and 49%, respectively.

Moreover, the protein transporter ABCA7 appears to exhibit a regulatory profile dependent on the flows of sterol, similar to that of the other members of the A subfamily, and in particular the ABCA1 transporter (Langman et al., BBR Com; 257(1999), 29-33; Laucken et al., PNAS, 97(2000) 817-822). There has indeed been observed by Kaminski et al. (supra) an increase in the expression of ABCA7 after incubation of human macrophages in the presence of acetylated low-density lipoproteins (AcLDL) which induce a sterol load, as well as a decrease in expression in the presence of the HDL3 cholesterol acceptor which causes a decrease in the sterol load.

Moreover, ABCA7 exhibits, like the other ABCA members, a degree of specialization of its tissue expression, the ABCA7 messenger being predominantly present in the hematopoietic tissues consisting of the lymphocytes, granulocytes, thymus, spleen, bone marrow or fetal tissues, whereas the expression of ABCA1 is predominant in the macrophages and the placenta, and that of ABCA4 is restricted in the retina (Rust et al., Nat. Genet, 22, (1999) 352-355).

All the data disclosed above, relating to the identity of the protein sequences, to the regulatory mechanism and the specificity of expression suggests that the ABCA7 gene constitutes another transporter of the A subfamily, and that it has a similar, or even redundant, function to that of the other transporters and in particular to that of the ABCA1 transporter. This transporter could therefore presumably act as mediator in the metabolism of lipids, and it is highly possible that it is, in the same way as the ABCA1 transporter, responsible for certain metabolic dysfunctions or deficiencies. Moreover, the specialization of the expression of the ABCA7 transporter presumably indicates that the latter plays a role in the transmembrane transport (export) of lipids in the hematopoietic tissues, and possibly in the lymphocyte signaling aA mechanisms of immunity, for example in the case of the pathogenesis of atherosclerosis as indicated by Kaminski et al. (Supra)

Although the expression of the human ABCA7 gene appears to be regulated according to the type of cell or the metabolic situation of a given cell type, the sequence(s) making it possible to regulate this gene were not known.

However, a need exists in the state of the art to identify these regulatory sequences for the following reasons:

a) These sequences are capable of being mutated in patients suffering from a pathology linked to a defect in the transport of lipids, possible substrates of the ABCA7 protein, or in patients who are likely to develop such pathologies.

The characterization of the regulatory sequences of the human ABCA7 gene would make it possible to detect mutations in patients, in particular to diagnose the individuals belonging to at-risk family groups. In addition, the isolation of these regulatory sequences would allow the complementation of the mutated sequence with a functional sequence capable of compensating for the metabolic dysfunctions induced by the mutation(s) diagnosed, by virtue of the construction of targeted therapeutic means, such as means intended for gene therapy.

b) The characterization of the regulatory sequences of the ABCA7 gene would make available to persons skilled in the art means capable of allowing the construction, by genetic engineering, and then the expression of defined genes in the cell types in which the ABCA7 gene is preferably expressed.

c) Moreover, some parts of the regulatory sequences of the ABCA7 gene could constitute constitutive promoter sequences with a high level of expression, of the type which will allow the construction of novel means for the expression of defined sequences in the cells, supplementing a range of means which already exist.

It has to be noted that despite the efforts undertaken, the regulatory sequences of the ABCA7 gene have so far remained completely unknown.

The inventors have now isolated and analyzed a human genomic DNA of 33.5 kb comprising the 46 exons of the open reading frame of the ABCA7 gene as well as the nontranscribed region of about 1.1 kb located on the 5′ side of exon 1, upstream of the transcriptional site +1, and comprising signals for regulating the human ABCA7 gene.

The inventors have also isolated and analyzed a murine genomic DNA of 20 Kb comprising the 45 exons of the open reading frame of the ABCA7 gene as well as the nontranscribed region of about 1.2 Kb in mice located on the 5′ side of exon 1, upstream of the transcription site +1, and comprising signals for regulating the murine ABCA7 gene.

General Definitions

The term “isolated” for the purposes of the present invention designates a biological material (nucleic acid or protein) which has been removed from its original environment (the environment in which it is naturally present).

For example, a polynucleotide present in the natural state in a plant or an animal is not isolated. The same polynucleotide separated from the adjacent nucleic acids in which it is naturally inserted in the genome of the plant or animal is considered as being “isolated”.

Such a polynucleotide may be included in a vector and/or such a polynucleotide may be included in a composition and remain nevertheless in the isolated state because of the fact that the vector or the composition does not constitute its natural environment.

The term “purified” does not require the material to be present in a form of absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

For the purposes of the present description, the expression “nucleotide sequence” may be used to designate either a polynucleotide or a nucleic acid. The expression “nucleotide sequence” covers the genetic material itself and is therefore not restricted to the information relating to its sequence.

The terms “nucleic acid”, “polynucleotide”, “oligonucleotide” or “nucleotide sequence” cover RNA, DNA or cDNA sequences or alternatively RNA/DNA hybrid sequences of more than one nucleotide, either in the single-chain form or in the duplex form.

The term “nucleotide” designates both the natural nucleotides (A, T, G, C) as well as the modified nucleotides which comprise at least one modification such as (1) an analog of a purine, (2) an analog of a pyrimidine, or (3) an analogous sugar, examples of such modified nucleotides being described, for example, in the PCT application No. WO 95/04 064.

For the purposes of the present invention, a first polynucleotide is considered as being “complementary” to a second polynucleotide when each base of the first nucleotide is paired with the complementary base of the second polynucleotide whose orientation is reversed. The complementary bases are A and T (or A and U), or C and G.

“Variant” of a nucleic acid according to the invention will be understood to mean a nucleic acid which differs by one or more bases relative to the reference polynucleotide. A variant nucleic acid may be of natural origin, such as an allelic variant which exists naturally, or may also be a nonnatural variant obtained, for example, by mutagenic techniques.

In general, the differences between the reference nucleic acid and the variant nucleic acid are small such that the nucleotide sequences of the reference nucleic acid and of the variant nucleic acid are very similar and, in many regions, identical. The nucleotide modifications present in a variant nucleic acid may be silent, which means that they do not alter the amino acid sequences encoded by said variant nucleic acid.

However, the changes in nucleotides in a variant nucleic acid may also result in substitutions, additions or deletions in the polypeptide encoded by the variant nucleic acid in relation to the peptides encoded by the reference nucleic acid. In addition, such nucleotide modifications in the coding regions may produce conservative or nonconservative substitutions in the amino acid sequence.

Preferably, the variant nucleic acids according to the invention encode polypeptides which substantially conserve the same function or biological activity as the polypeptide of the reference nucleic acid or, alternatively, the capacity to be recognized by antibodies directed against the polypeptides encoded by the initial nucleic acid.

Some variant nucleic acids will thus encode mutated forms of the polypeptides whose systematic study will make it possible to deduce structure-activity relationships of the proteins in question. Knowledge of these variants in relation to the disease studied is essential since it makes it possible to understand the molecular cause of the pathology.

“Fragment” will be understood to mean a reference nucleic acid according to the invention, a nucleotide sequence of reduced length relative to the reference nucleic acid and comprising, over the common portion, a nucleotide sequence identical to the reference nucleic acid.

Such a nucleic acid “fragment” according to the invention may be, where appropriate, included in a larger polynucleotide of which it is a constituent.

Such fragments comprise, or alternatively consist of, oligonucleotides ranging in length from 20 to 25, 30, 40, 50, 70, 80, 100, 200, 500,1000 or 1500 consecutive nucleotides of a nucleic acid according to the invention.

“Biologically active fragment” of a nucleic acid for regulating transcription according to the invention is understood to mean a nucleic acid capable of modulating the transcription of a DNA sequence placed under its control. Such a biologically active fragment comprises a core promoter and/or a regulatory element, as defined in the present description.

“Regulatory nucleic acid” according to the invention is understood to mean a nucleic acid which activates and/or regulates the expression of a DNA sequence selected and placed under its control.

“Promoter” is understood to mean a DNA sequence recognized by the proteins of the cell which are involved in the initiation of the transcription of a gene. The core promoter is the minimum regulatory nucleic acid capable of initiating the transcription of a defined DNA sequence which is placed under its control. In general, the core promoter consists of a genomic DNA region upstream of the site for initiation of transcription where there is very often present a CAAT sequence (where one or more transcriptional protein factors bind) as well as, except in rare cases such as in some housekeeping genes, the TATA or “TATA box” sequence or a related box. It is at the level of this box that RNA polymerase binds as well as one or more transcription factors, such as the “TATA” box binding proteins (TBPs).

A nucleotide sequence is “placed under the control” of a regulatory nucleic acid when this regulatory nucleic is located, relative to the nucleotide sequence, in such a manner as to control the initiation of transcription of the nucleotide sequence by an RNA polymerase.

“Regulatory element” or “regulatory sequence” for the purposes of the invention, is understood to mean a nucleic acid comprising elements capable of modulating transcription initiated by a core promoter, such as binding sites for various transcription factors, “enhancer” sequences for increasing transcription or “silencer” sequences for inhibiting transcription.

“Enhancer” sequence is understood to mean a DNA sequence included in a regulatory nucleic acid capable of increasing or stimulating transcription initiated by a core promoter.

“Silencer” sequence is understood to mean a DNA sequence included in a regulatory acid capable of decreasing or inhibiting transcription initiated by a core promoter.

Regulatory elements may be present outside the sequence located on the 5′ side of the site for initiation of transcription, for example in the introns and exons, including in the coding sequences.

The core promoter and the regulatory element may be “specific to one or more tissues” if they allow transcription of a defined DNA sequence placed under their control, preferably in certain cells (for example tissue-specific cells), that is to say either exclusively in the cells of certain tissues, or at different levels of transcription according to the tissues.

“Transcription factor” is understood to mean proteins which preferably interact with elements for regulating a regulatory nucleic acid according to the invention, and which stimulate or on the contrary repress transcription. Some transcription factors are active in the form of monomers, others being active in the form of homo- or heterodimers.

The term “modulation” refers to either a positive regulation (increase, stimulation) of transcription, or a negative regulation (decrease, inhibition, blockage) of transcription.

The “percentage identity” between two nucleotide or amino acid sequences, for the purposes of the present invention, may be determined by comparing two sequences aligned optimally, through a window for comparison.

The portion of the nucleotide or polypeptide sequence in the window for comparison may thus comprise additions or deletions (for example “gaps”) relative to the reference sequence (which does not comprise these additions or these deletions) so as to obtain an optimum alignment of the two sequences.

The percentage is calculated by determining the number of positions at which an identical nucleic base or an identical amino acid residue is observed for the two sequences (nucleic or peptide) compared, and then by dividing the number of positions at which there is identity between the two bases or amino acid residues by the total number of positions in the window for comparison, and then multiplying the result by 100 in order to obtain the percentage sequence identity.

The optimum sequence alignment for the comparison may be achieved using a computer with the aid of known algorithms contained in the package from the company WISCONSIN GENETICS SOFTWARE PACKAGE, GENETICS COMPUTER GROUP (GCG), 575 Science Doctor, Madison, Wis.

By way of illustration, it will be possible to produce the percentage sequence identity with the aid of the BLAST software (versions BLAST 1.4.9 of March 1996, BLAST 2.0.4 of February 1998 and BLAST 2.0.6 of September 1998), using exclusively the default parameters (Altschul et al, J. Mol. Biol. (1990) 215: 403-410; Altschul et al, Nucleic Acids Res. (1997) 25: 3389-3402). Blast searches for sequences similar/homologous to a reference “request” sequence, with the aid of the Altschul et al. (Supra) algorithm. The request sequence and the databases used may be of the peptide or nucleic type, any combination being possible.

“High stringency hybridization conditions” for the purposes of the present invention will be understood to mean the following conditions:

1—Membrane Competition and Prehybridization:

Mix: 40 μl salmon sperm DNA (10 mg/ml)

+40 μl human placental DNA (10 mg/ml)

Denature for 5 min at 96° C., then immerse the mixture in ice.

Remove the 2× SSC buffer and pour 4 ml of formamide mix into the hybridization tube containing the membranes.

Add the mixture of the two denatured DNAs.

Incubate at 42° C. for 5 to 6 hours, with rotation.

2—Labeled Probe Competition:

Add to the labeled and purified probe 10 to 50 μl Cot I DNA, depending on the quantity of nonspecific hybridizations.

Denature for 7 to 10 min at 95° C.

Incubate at 65° C. for 2 to 5 hours.

3-Hybridization:

Remove the prehybridization mix.

Mix 40 μl salmon sperm DNA +40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4-Washes:

One wash at room temperature in 2× SSC, to rinse.

Twice 5 minutes at room temperature 2× SSC and 0.1% SDS.

Twice 15 minutes 0.1× SSC and 0.1% SDS at 65° C.

Envelope the membranes in Saran wrap and expose.

The hybridization conditions described above are adapted to hybridization, under high stringency conditions, of a molecule of nucleic acid of varying length from 20 nucleotides to several hundreds of nucleotides.

It goes without saying that the hybridization conditions described above may be adjusted as a function of the length of the nucleic acid whose hybridization is sought or of the type of labeling chosen, according to techniques known to persons skilled in the art.

Suitable hybridization conditions may for example be adjusted to the teaching contained in the manual by HAMES and HIGGINS (1985) (Nucleic acid Hybridization: A Practical Approach, Hames and Higgins Ed., IRL Press, Oxford) or in the manual by F. AUSUBEL et al (1999) (Currents Protocols in Molecular Biology, Green Publishing Associates and Wiley Interscience, N.Y).

“Transformation” for the purposes of the invention is understood to mean the introduction of a nucleic acid (or of a recombinant vector) into a host cell. The term “transformation” also covers a situation in which the genotype of a cell has been modified by an exogenous nucleic acid, and that this cell thus transformed expresses said exogenous nucleic acid, for example in the form of a recombinant polypeptide or in the form of a sense or antisense nucleic acid.

“Transgenic animal” for the purposes of the invention is understood to mean a nonhuman animal, preferably a mammal, in which one or more cells contain a heterologous nucleic acid introduced by virtue of human intervention, such as by transgenesis techniques well known to persons skilled in the art. The heterologous nucleic acid is introduced directly or indirectly into the cell or the precursor of the cell, by genetic engineering such as microinjection or infection with a recombinant virus. The heterologous nucleic acid may be integrated into the chromosome or may be provided in the form of DNA replicating extrachromosomally.

NUCLEIC ACID FOR REGULATING THE ABCA7 GENE

Using BAC-type vector libraries prepared from human and murine genomic material, the inventors succeeded in isolating a nucleic acid for regulating the human and murine ABCA7 genes.

The inventors determined, by comparative analysis of the human and murine genomic sequences, a regulatory nucleic acid comprising in particular two regulatory modules conserved in humans and mice. The inventors therefore determined that the nucleic acid for regulating transcription of the ABCA7 gene, when it is most broadly defined, consists of a polynucleotide comprising, from the 5′ end to the 3′ end:

a nontranscribed region of about 1.2 kb located upstream of the site for initiation of transcription of the ABCA7 gene, and

the partial sequence of the first exon of the ABCA7 gene.

In its broadest definition, the nucleic acid for regulating transcription of the ABCA7 gene comprises all the nucleotide regions as defined above and is identified in the sequence SEQ ID No. 1 according to the invention.

Thus, a first subject of the invention consists of a nucleic acid comprising a polynucleotide having at least 20 consecutive nucleotides of the nucleotide sequence SEQ ID No. 1, or a nucleic acid having a complementary sequence.

The region of about 1.1 Kb located upstream of the site for initiation of transcription of the ABCA7 gene, and comprising the core promoter and multiple elements for regulating transcription is also included in the sequence identified as SEQ ID No. 2 according to the invention.

More precisely, the nucleotide at position 1 of the sequence SEQ ID No. 2 is the nucleotide at position −1111, relative to the site for initiation of transcription of the ABCA7 gene.

According to a second aspect, the invention relates to a nucleic acid comprising a polynucleotide having at least 20 consecutive nucleotides having the nucleotide sequence SEQ ID No. 2, or a nucleic acid having a complementary sequence.

As already specified above, the nucleic acid for regulating the transcription of the ABCA7 gene having the sequence SEQ ID No. 1 also comprises, in addition to a nontranscribed 5′ regulatory region, the 5′ part of the first exon of the human ABCA7 gene.

The partial sequence of the first exon of the ABCA7 gene is defined as the sequence SEQ ID No. 3.

According to a third aspect, the invention relates to a nucleic acid comprising a polynucleotide having at least 20 consecutive nucleotides having the nucleotide sequence SEQ ID No. 3, or a nucleic acid having a complementary sequence.

Preferably, a nucleic acid according to the invention will be in isolated and/or purified form.

Also forming part of the invention is any “biologically active” fragment of a nucleic acid as defined above.

According to yet another aspect, the invention relates to a nucleic acid having at least 80% nucleotide identity with a nucleic acid as defined above.

In particular, this nucleic acid may be of murine origin, and consists of a polynucleotide having the nucleotide sequence SEQ ID NO: 4 comprising from the 5′ to the 3′ end:

a nontranscribed region of about 1.2 Kb located upstream of the site for initiation of transcription of the murine ABCA7 gene, and

the partial sequence of the first exon of the ABCA7 gene.

The region of about 1.2 Kb located upstream of the site for initiation of transcription of the ABCA7 gene, and comprising the core promoter and multiple elements for regulating transcription, is also included in the sequence identified as SEQ ID NO: 5 according to the invention.

The invention also includes a nucleic acid characterized in that it hybridizes, under high stringency conditions, with any of the nucleic acids according to the invention.

The invention also relates to a nucleic acid having at least 80%, advantageously 90%, preferably 95% and most preferably 98% nucleotide identity with a nucleic acid comprising at least 20 consecutive nucleotides of a polynucleotide chosen from the group consisting of the nucleotide sequences SEQ ID No. 1 to SEQ ID No. 5.

Detailed Analysis of the Sequences SEQ ID No.2 and SEQ ID No. 5

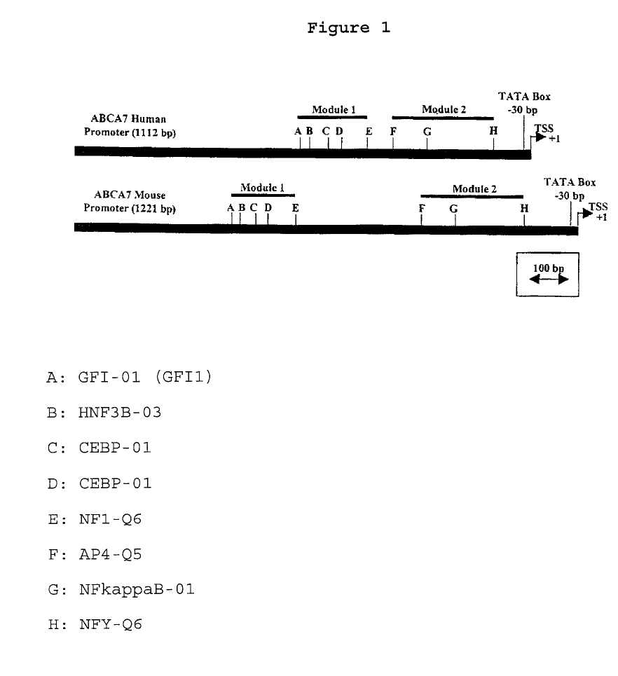

According to a principal characteristic, the nucleic acid having the sequence SEQ ID No. 2, included in the nucleic acid for regulating the human ABCA7 gene having the sequence SEQ ID No. 1, comprises the constituent elements of a core promoter, respectively a degenerate “TATA” box (TTAAG) located 30 bp upstream of the site of initiation of transcription. Likewise, a degenerate “TATA” box (TTAAA) is located 30 bp upstream of the site of initiation of transcription, on the murine nucleic acid having the sequence SEQ ID NO: 5, included in the nucleic acid for regulating the murine ABCA7 gene having the sequence SEQ ID NO: 4. The “TATA” boxes on the promoters of the human and murine ABCA7 genes as well as the position of the sites of initiation of transcription are represented in FIG. 1.

The regulatory sequences SEQ ID No. 2 and SEQ ID No. 5 also comprise numerous binding sites for various transcription factors capable of positively or negatively regulating the activity of the core promoter.

Thus, the various sequences characteristic of the sites for the binding of various transcription factors in the sequences SEQ ID No. 2 and SEQ ID No. 5 were identified by the inventors in the manner detailed below.

The sequences SEQ ID No. 2 and SEQ ID No. 5 were used as reference sequences and treated according to the algorithms of the MatInspector software packages (Quandt et al., Nucl Acid Res (1995) 23(23), 4878-4884) and compared with the data stored in several databases such as Transfac and the presence as well as the location of the various sites characteristic of the sequences SEQ ID No 2 and 5, and particularly the sites for the binding of the transcription factors were determined according to methods well known to persons skilled in the art.

More particularly, a detailed analysis was carried out using the software packages NNPP (Reese et al. J. Comput Biol. (1997) 4(3) 311-23), TSSG and TSSW (Soloryev et al., Ismb (1995), 5, 294-302), on the 1.1 kb and 1.2 Kb upstream of the site of initiation of the sequences SEQ ID No. 2 and 5, respectively, making it possible to identify 193 and 233 putative sites for binding to the transcription factors, in humans and mice during the first stage of the search. These are collated in Tables 1 and 2. After compiling and filtering as described above, and comparing the human and murine regulatory sequences, two modules common to the human and murine regulatory sequences were determined, and 5 and 3 possible sites for binding of various transcription factors were selected in the modules 1 and 2, respectively, on the human and murine sequences. The position with the filtration scores for the sites for binding to the transcription factors identified in the 1111 bp of the sequence SEQ ID No. 2 according to the invention, as well as in the 1220 bp of the sequence SEQ ID NO: 5 according to the invention, are presented in Table 3. The various binding sites are also schematically represented in FIG. 1.

The positions of the starting nucleotides in each of the sites for binding to the transcription factors are designated with reference to the numbering of the nucleotides of the sequences SEQ ID No. 2 and No: 5 relative to the site of initiation of transcription +1, contained in the sequences SEQ ID No. 1 and No. 4, as represented in FIG. 1.

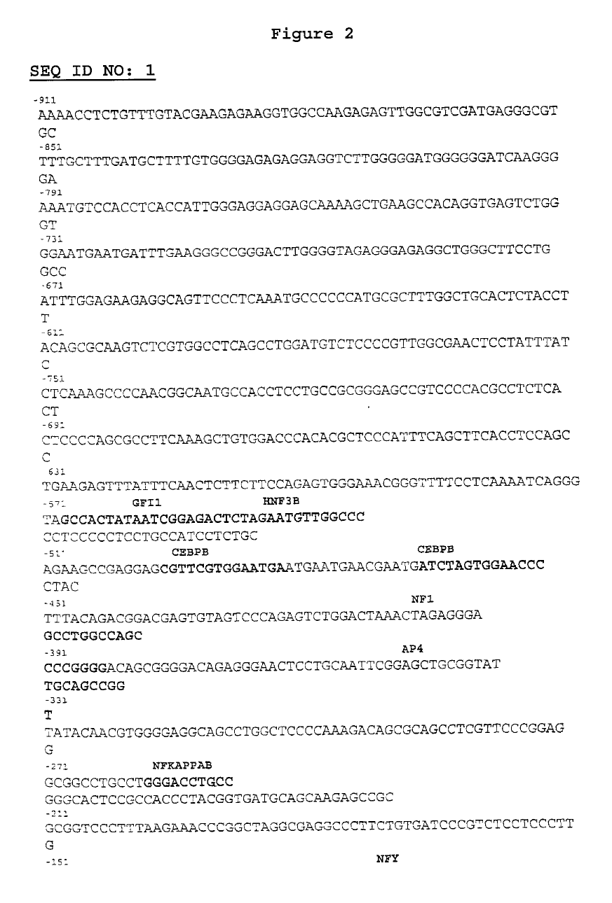

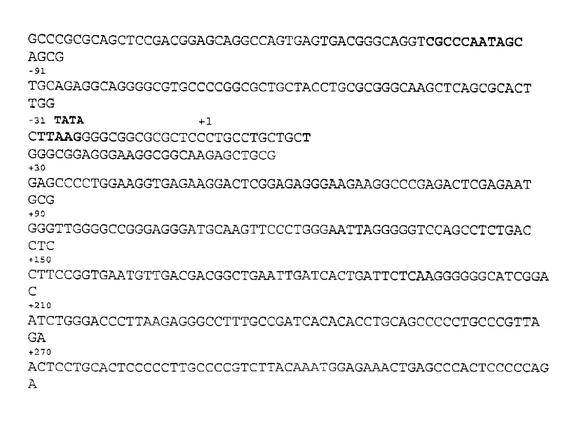

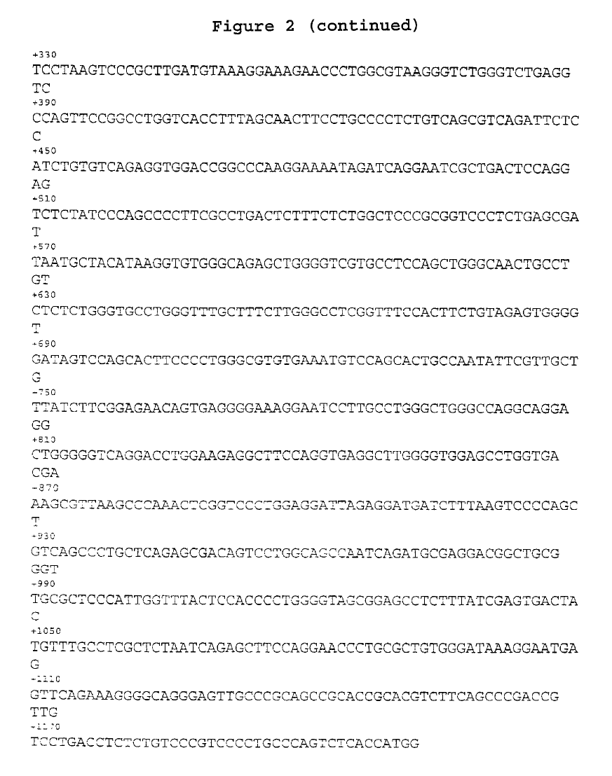

FIG. 2 represents the sequence SEQ ID NO: 1, which contains the sequence SEQ ID No. 2. The first nucleotide at position 5′ of the sequence of FIG. 2 is also the first nucleotide at position 5′ of one of the nucleotide sequences SEQ ID No. 1 and SEQ ID No. 2. In FIG. 2, the sites for binding to the transcription factors are illustrated in bold characters which delimit their respective positions, and their respective designations are indicated above each of the corresponding boxes. The numbering of the nucleotides of the sequence represented in FIG. 2 was carried out relative to the site of initiation of transcription, numbered “+1”, the nucleotide 5′ of the nucleotide +1 being itself numbered “−1”.

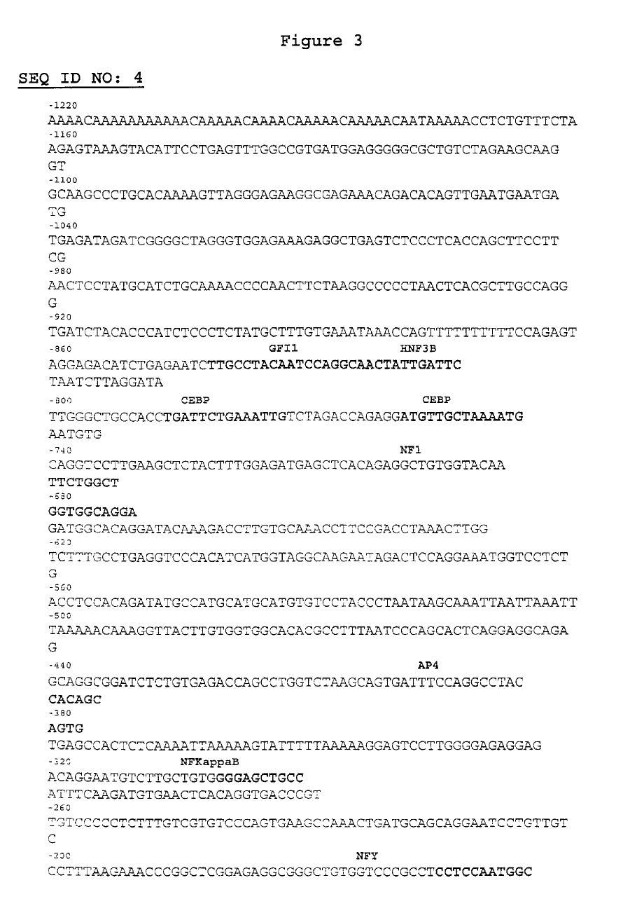

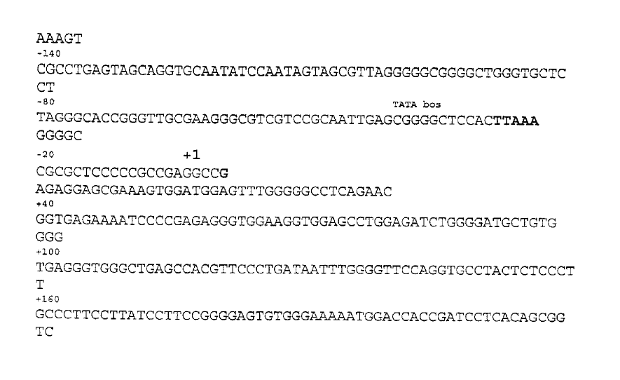

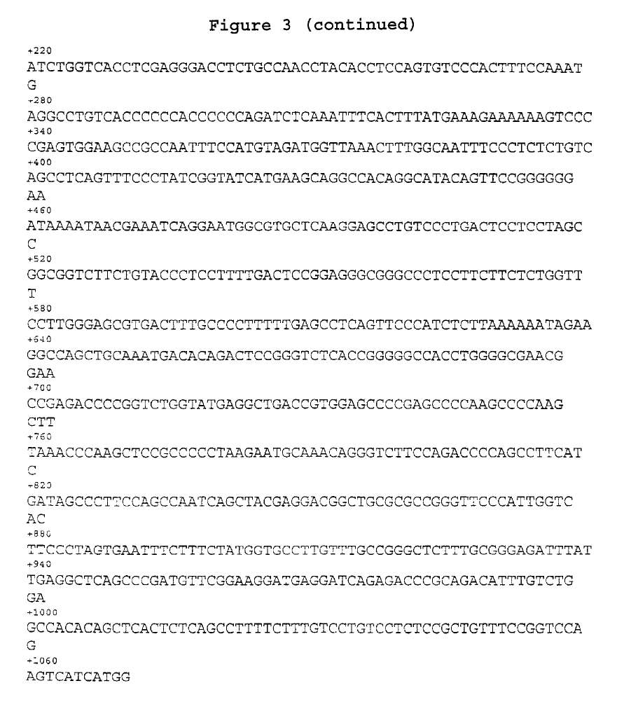

FIG. 3 represents the sequence SEQ ID NO: 4, which contains the sequence SEQ ID No. 5. The first nucleotide at position 5′ of the sequence of FIG. 3 is also the first nucleotide at position 5′ of one of the nucleic sequences SEQ ID No. 4 and SEQ ID No. 5. In FIG. 3, the sites for binding to the transcription factors are illustrated in bold characters which delimit their respective positions, and their respective designations are indicated above each of the corresponding boxes. The numbering of the nucleotides of the sequence represented in FIG. 3 is relative to the site of initiation of transcription, numbered “+1”, the nucleotide in 5′ of the nucleotide +1 being itself numbered “−1”.

The genomic analysis of the nucleic acids regulating the human and murine sequences SEQ ID NO: 2 and 5, revealed two regulatory modules which were denoted module 1 and module 2, and are particularly conserved in humans and mice. These two regulatory modules comprise ubiquitous transcription factor binding sites, such as NF1, NFY and AP4, as well as sites for binding of transcription factors specific to the liver such as CEBP and HNF3B. This is compatible with the experimental expression data presented in Example 3 below, and provided by Kaminski et al. (supra), which show expression of the ABCA7 gene in human fetal hepatic tissues.

The two regulatory modules conserved in mice and humans also comprise sites for binding of transcription factors such as GFI1 and NFkappaB (NFkB), which are essentially present in the lymphatic organs.

The description of the characteristics of the sites for binding to each of the transcription factors designated in FIGS. 2 and 3 as well as in Table 3 can be easily found by persons skilled in the art. A short description of some of them is made below.

NFI factor:

The binding characteristics of the NF1 factor can be found in particular in the following entries of the Medline database: 88319941, 91219459, 86140112, 87237877, 90174951,89282387, 90151633,892618136, 86274639,87064414, 89263791. The NF1 factor recognizes the following palindromic sequence: “TGGCANNNTGCCA (NNTTGGCNNNNNNNNCCNN)” which is present in viral and cellular promoters and at the level of the origin of replication of type 2 adenoviruses. These proteins are capable of activating transcription and replication. They bind to DNA in the form of a homodimer.

NFY Factor:

The NFY factor is in particular described in entry No. P25.208 of the Swissprot database. It is a factor which recognizes a CCAAT″ unit in the promoter sequences such as those of the gene encoding type 1 collagen, albumin and -actin. It is a stimulator of transcription.

AP4 Factor:

Persons skilled in the art will be able to advantageously refer to the articles corresponding to the following entries of the Medline database: 2123466, 2833704, 8530024. The AP4 factor has a domain for binding to DNA of the “helix loop helix” (bHLH) type as well as two dimerization domains. The consensus sit e of the AP4 factor is the following “CWCAGCTGGN”, and the latter generally overlaps with a binding site for the AP1 factor.

CEBP

The characteristics for binding to the CEBP factor may be found in particular in the following entries of the Medline database: 93315489, 91248826, 94193722, 93211931, 92390404, 90258863, 94088523, 90269225 and 96133958. It is an important transcription activator in the regulation of genes involved in the immune and inflammatory responses. It binds specifically to an IL-1 response element in the gene for IL-6. It presumably plays a role in the regulation of the acute phase of the inflammation and in hematopoiesis. The consensus recognition site is the following: “T(T/G) NNGNAA(T/G)”.

HNF3B Factor:

Persons skilled in the art will be able to advantageously refer to the article by Overdier et al. (1994, Mol. Cell Biol. 14: 2755-2766), as well as to the following entries of the Medline database: 91352065, 91032994, 92345837, 89160814, 91187609, 91160974, 91029477, 94301798 and 94218249. This transcription factor acts as activator of numerous genes in the liver such as the AFT gene and the genes for albumin and tyrosine aminotransferase and interacts with cis-acting regulatory regions of these genes.

GFI1

The characteristics for binding to the GFI1 factor may be found in particular in the following entries of the Medline database: 10762661, 9931446, 9571157, 9285685, 9070650 and 7789186. The GFI1 gene encodes a zinc finger protein involved in the transcriptional regulation and more particularly in the interleukin-2 signaling pathway. The consensus recognition site is the following: “NNNNNNAAATCANNGNNNNNNN”

NFkappa-B Factor:

Persons skilled in the art will be able to advantageously refer to the articles corresponding to the following entries of the Medline database: 95369245, 91204058,94280766, 89345587, 93024383,88248039, 94173892,91088538, 91239561, 91218850,92390404, 90156535,93377072, 92097536, 93309429, 93267517, 92037544, 914266911, 91105848 and 95073993. The NFkappa-B factor is a heterodimer consisting of a first subunit of 50 kDa and a second subunit of 65 kDa. Two heterodimers may form a labile tetramer. Its binding to DNA depends on the presence of zinc (Zn++). It may be induced by numerous agents such as TNF, PKA or PKC. It is a key regulator of genes involved in responses to infection, inflammation and stress.

An essential characteristic of the regulatory nucleic acid according to the invention, and more particularly of the sequence located upstream of the site of initiation of transcription included both in the sequence SEQ ID No. 2 and in the sequence SEQ ID No. 5 is the presence of motifs characteristic of putative sites for binding to transcription factors involved in the gene expression of the T lymphocytes, such as the transcription factors CEBP, NFKB and GFI1.

GFI1 is a protooncogene which encodes a zinc finger nuclear protein involved in the cytokine signaling pathway and in the clonal amplification of the T cells (Zweidler-McKay, et al., Mol. Cell. Biol. (1966), 16(8), 4024-4034). The transcription factor GFI1 which acts as a transcriptional repressor of the genes which inhibit the activation of the T cells and oncogenesis. It is specifically present in the thymus, the spleen and the T lymphocytes.

The transcription factors CEBP and NFkappaB which are expressed in the thymus, the spleen and the T lymphocytes are well known to persons skilled in the art and act in cooperation in the mediation of the induction of the expression of the genes of the T lymphocytes (Runch et al., 1994) and of the HepB3 cells (Shimizu et al., Gene, (1994) 149, 305-310).

The positions of the starting nucleotides, relative to the site of initiation of transcription which are at −498 and −469 for the CEBP sites, and at −260 for the NFkB site, on the human regulatory module, and at −787 and −760, for the CEBP sites, and at −301 for the NFKB site, show that the two regulatory sites are more distant in the mouse promoter. However, it is probable that the two sites are closer in a three-dimensional structure so as to allow coactivation by the two factors CEBP and NFkB.

The presence of these potential sites for binding to CEBP and to NFkB in a manner conserved in humans and mice on the regulatory nucleic acids according to the invention is compatible with the observation according to which the expression of the gene encoding the human ABCA7 protein is predominant in the hematopoietic tissues and the T lymphocytes, and is thought to be most probably involved in cellular mediation of immunity, in particular in the pathogenesis of atherosclerosis (Kaminski et al., Supra).

As already mentioned above, the invention relates to a nucleic acid comprising a polynucleotide having at least 20 consecutive nucleotides of one of the nucleotide sequences SEQ ID No. 1 or 2, and SEQ ID No. 4 or 5, as well as a nucleic acid having a complementary sequence.

Included in the above definition are the nucleic acids comprising one or more “biologically active” fragments of one of the sequences SEQ ID No. 1 or 2, and SEQ ID No. 4 or 5. Persons skilled in the art can easily obtain biologically active fragments of these sequences, by referring in particular to Table 3 above as well as to FIGS. 2 and 3 in which the various characteristic units of the sequence for regulating the ABCA7 gene are present. Persons skilled in the art can thus obtain such biological active fragments by total or partial chemical synthesis of the corresponding polynucleotides or by causing restriction endonucleases to act in order to obtain desired DNA fragments, it being possible for the restriction sites present on the sequences SEQ ID No. 1 to SEQ ID No. 5 to be easily found from the sequence information, with the aid of common software packages for restriction mapping such as GCG version 9.1 map module.

The production of defined nucleic acid fragments with the aid of restriction endonucleases is for example described in the manual by Sambrook et al., (Molecular cloning: a laboratory manual, 2ed. Cold Spring Harbor Laboratory, Cold spring Harbor, N.Y. (1989).

The invention therefore also relates to a nucleic acid as defined above, which is capable of modulating the transcription of a polynucleotide placed under its control.

According to a first preferred embodiment, a biologically active fragment of a nucleic acid for regulating transcription according to the invention comprises a first conserved module (module 2) which comprises the core promoter (TATA box) ranging from the nucleotide at position −1 to the nucleotide at position −390, relative to the site of initiation of transcription, the first nucleotide transcribed being the nucleotide at position 1112 of the nucleotide sequence SEQ ID No. 1, or the nucleotide at position 1221 of the nucleotide sequence SEQ ID NO: 4.

According to a second embodiment, a biologically active fragment of a nucleic acid for regulating transcription according to the invention comprises the conserved modules 1 and 2 (FIG. 1) from the nucleotide at position −1 to the nucleotide at position −860, relative to the site of initiation of transcription, the first nucleotide transcribed being the nucleotide at position 1112 of the nucleotide sequence SEQ ID No. 1, or the nucleotide at position 1221 of the nucleotide sequence SE ID NO: 4.

According to a third embodiment, such a biologically active fragment of an acid for regulating transcription according to the invention comprises, in addition to the core promoter and the proximal regulatory elements, also other regulatory elements such as the various sites GFI1, HNF3B, CEBPB, NF1 and extends from the nucleotide at position −1 to the nucleotide at position −1111, relative to the site of initiation of transcription, the first nucleotide transcribed being the nucleotide at position 1112 of the nucleotide sequence SEQ ID No. 1, and to the nucleotide at position −1220, relative to the site of initiation of transcription, the first nucleotide transcribed being the nucleotide at position 1221 of the nucleotide sequence SEQ ID No. 4.

Analysis of Exon 1

The applicant has also identified the nucleotide sequences located downstream of the site of initiation of transcription and corresponding to the 5′ end of exon 1, human and murine genes encoding the ABCA7 protein.

More precisely, the 5′ end of exon 1, having a size of 1210 nucleotides, starts with the nucleotide at position 1112 of the sequence SEQ ID No. 1 and ends with the nucleotide at position 2322 of the sequence SEQ ID No. 1. The 5′ end of exon 1 is identified as the sequence SEQ ID No. 3 and the complete sequence of exon 1 is identified as the sequence SEQ ID No. 6.

Exon 1 contains the beginning of the open reading phase of the human ABCA7 gene, the nucleotide A of the ATG codon being located at position 1208 of the sequences SEQ ID No. 3 and 6. Exon 1 encodes the polypeptide having the sequence SEQ ID No. 7.

Exon 1 is likely to contain elements for regulating the expression of the ABCA7 gene, in particular elements of the amplifying enhancer type and/or elements of the silencer or repressor type.

Consequently, a nucleic acid for regulating transcription according to the invention may also contain, in addition to biologically active fragments of the sequence SEQ ID No. 1, also nucleotide fragments, or even the entire sequences SEQ ID No. 2 to SEQ ID No. 3 and 6.

The nucleotide sequences SEQ ID No. 1 to SEQ ID No. 3 and 6, as well as their fragments, may in particular be used as nucleotide probes or primers for detecting the presence of at least one copy of the ABCA7 gene in a sample, or for amplifying a defined target sequence in the sequence for regulating the ABCA7 gene.

The subject of the invention is therefore also a nucleic acid having at least 80% nucleotide identity with a nucleic acid as defined above, in particular obtained from one of the sequences SEQ ID No. 1 to SEQ ID No. 3 and 6.

The invention also relates to a nucleic acid which hybridizes, under high stringency conditions, with any one of the nucleic acids according to the invention, in particular a nucleic acid obtained from a sequence chosen from the sequences SEQ ID No. 1 to SEQ ID No. 3 and 6.

The invention also relates to a nucleic acid as defined above and characterized, in addition, in that it is capable of modulating the transcription of a polynucleotide of interest placed under its control.

According to a first aspect, such a nucleic acid is capable of activating the transcription of the polynucleotide of interest placed under its control.

According to a second aspect, a regulatory nucleic acid according to the invention may be characterized in that it is capable of inhibiting the transcription of the polynucleotide of interest placed under its control.

Preferably, a nucleic acid for regulating transcription according to the invention, when it is suitably located relative to a polynucleotide of interest whose expression is sought, will allow the transcription of said polynucleotide of interest, either constitutively or inducibly.

The inducible character of the transcription initiated by a regulatory nucleic acid according to the invention may be conferred by one or more of the regulatory elements which it contains, for example the presence of one or more sites as defined above in the sequence SEQ ID No. 1 or SEQ ID No. 2.

Furthermore, a tissue-specific expression of the polynucleotide of interest may be sought by placing this polynucleotide of interest under the control of a regulatory nucleic acid according to the invention which is capable, for example, of initiating the transcription of this polynucleotide of interest specifically in certain categories of cells, for example cells of the hematopoietic tissue, such as the peripheral leukocytes, thymus cells, spleen cells and bone marrow.

Preferably, a regulatory nucleic acid according to the invention may comprise one or more “discrete” regulatory elements such as enhancer and silencer elements. In particular, such a regulatory nucleic acid may comprise one or more potential sites for binding to the transcription factors as defined in FIG. 2.

A regulatory acid according to the invention also includes a sequence which does not comprise the core promoter, that is to say the sequence ranging from the nucleotide at position −1 to the nucleotide at position −25, relative to the site of initiation of transcription.

Such a regulatory nucleic acid will then preferably comprise a so-called “heterologous” core promoter, that is to say a polynucleotide comprising a “TATA” box and a “homeobox” not derived from the nucleic acid for regulating the ABCA7 gene.

Also forming part of the invention is a nucleic acid for regulating transcription comprising all or part of the sequence SEQ ID No. 1 which has been modified, for example, by addition, deletion or substitution of one or more nucleotides. Such modifications may modulate the transcriptional activity by causing an increase or on the contrary a decrease in the activity of the promoter or of the regulatory element.

Such a modification may also affect the tissue specificity of the promoter or of the regulatory element. Thus, for example, a regulatory nucleic acid according to the invention may be modified so as to stimulate transcription in only one of the tissues in which it is naturally expressed.

An acid for regulating transcription according to the invention may also be modified and be rendered inducible by a particular compound, for example by creating in the sequence an inducible site by a given therapeutic compound.

The modifications in a sequence comprising all or part of the sequence SEQ ID No. 1 and comprising the promoter or a regulatory element may be carried out with methods well known to persons skilled in the art, such as mutagenesis. The activity of the modified promoter or regulatory element may then be tested, for example by cloning the modified promoter upstream of a reporter gene, by transfecting the resulting DNA construct into a host cell and by measuring the level of expression of the reporter gene in the transfected host cell. The activity of the modified promoter can also be analyzed in vivo in transgenic animals. It is also possible to construct libraries of modified fragments which may be screened using functional tests in which, for example, only the promoters or regulatory elements having the desired activity will be selected.

Such tests may be based, for example on the use of reporter genes conferring resistance to defined compounds, for example to antibiotics. The selection of cells having a regulatory nucleic acid/reporter gene construct and containing a promoter or a regulatory element having a desired modification may then be isolated by culturing host cells transformed with such a construct in the presence of the defined compound, for example of the defined antibiotic.

The reporter gene may also encode any protein which is easily detectable, for example an optically detectable protein such as luciferase.

Consequently, the subject of the invention is also a nucleic acid comprising:

a) a nucleic acid for regulating transcription as defined above; and

b) a polynucleotide of interest encoding a polypeptide or a nucleic acid of interest.

According to a first aspect, the polynucleotide of interest whose transcription is desired encodes a protein or a peptide. The protein may be of any type, for example a protein of therapeutic interest, including cytokines, structural proteins, receptors or transcription factors. For example, in the case where transcription specifically in certain tissues is desired, such as for example in cells of the hematopoietic tissue, that is to say of the spleen, of the bone marrow, or in the peripheral leukocytes, the nucleic acid regulating transcription will advantageously comprise a nucleic acid ranging from the nucleotide at position −1 to the nucleotide at position −1111, relative to the site of initiation of transcription of the sequence SEQ ID No. 1 or 2, and ranging from the nucleotide at position −1 to the nucleotide at position −1220 SEQ ID No. 4 or 5.

In this case, the polynucleotide of interest will encode a gene involved in combating inflammation, such as a receptor for cytokines or for a superoxide dismutase. If an antitumor effect is desired, it will then be sought to stimulate the number and the activation of the cytotoxic T lymphocytes specific for a given tumor antigen.

In another embodiment, a regulatory nucleic acid according to the invention will be used in combination with a polynucleotide of interest encoding the ABCA7 protein.

As already mentioned, the polynucleotide of interest may also produce a nucleic acid, such as an antisense nucleic acid specific for a gene, the inhibition of whose translation is sought.

According to another aspect, the polynucleotide of interest whose 3 transcription is regulated by the regulatory nucleic acid is a reporter gene, such as any gene encoding a detectable protein.

Among the preferred reporter genes, there may be mentioned in particular the gene for luciferase, for -galactosidase (LacZ), for chloramphenicol acety-transferase (CAT) or any gene encoding a protein conferring resistance to a particular compound, in particular to an antibiotic.

Recombinant Vectors

The term “vector” for the purpose of the present invention will be understood to mean a circular or linear DNA or RNA molecule which is either in single-stranded or double-stranded form.

According to a first embodiment, a recombinant vector according to the invention is used in order to amplify the regulatory nucleic acid according to the invention which is inserted therein after transformation or transfection of the desired cellular host.

According to a second embodiment, it corresponds to expression vectors comprising, in addition to a regulatory nucleic acid in accordance with the invention, sequences whose expression is sought in a host cell or in a defined multicellular organism.

According to an advantageous embodiment, a recombinant vector according to the invention will comprise in particular the following elements:

(1) a regulatory nucleic acid according to the invention;

(2) a polynucleotide of interest comprising a coding sequence included in the nucleic acid to be inserted into such a vector, said coding sequence being placed in phase with the regulatory signals described in (1); and

(3) appropriate sequences for initiation and termination of transcription.

In addition, the recombinant vectors according to the invention may include one or more origins for replication in the cellular hosts in which their amplification or their expression is sought, markers or selectable markers.

By way of example, the bacterial promoters may be the Lacl or LacZ promoters, the T3 or T7 bacteriophage RNA polymerase promoters, the lambda phage PR or PL promoters.

The promoters for eukaryotic cells will comprise the HSV virus thymidine kinase promoter or alternatively the mouse metallothionein-L promoter.

Generally, for the choice of a suitable promoter, persons skilled in the art can advantageously refer to the book by Sambrook et al. (1989) cited above or to the techniques described by Fuller et al. (1996; Immunology in Current Protocols in Molecular Biology).

When the expression of the genomic sequence of the ABCA7 gene is sought, use will preferably be made of the vectors capable of containing large insertion sequences. In this particular embodiment, bacteriophage vectors such as the P1 bacteriophage vectors such as the vector p158 or the vector p158/neo8 described by Sternberg (Trends Genet., (1992) 8: 1-16; Mamm. Genome (1994) 5: 397-404) will be preferably used.

The preferred bacterial vectors according to the invention are for example the vectors pBR322(ATCC37017) or alternatively vectors such as pAA223-3 (Pharmacia, Uppsala, Sweden), and pGEM1 (Promega Biotech, Madison, Wis., UNITED STATES).

There may also be cited other commercially available vectors such as the vectors pQE70, pQE60, pQE9 (Qiagen), psiX174, pBluescript SA, pNH8A, pNH16A, pNH18A, pNH46A, PWLNEO, pSV2CAT, pOG44, pXTI, pSG(Stratagene).

These may also include the recombinant vector PXP1 described by Nordeen S K et al. (Bio Techniques, (1988), 6: 454-457).

They may also be vectors of the Baculovirus type such as the vector pVL1392/1393 (Pharmingen) used to transfect cells of the Sf9 line (ATCC No. CRL 1711) derived from Spodoptera frugiperda.

They may also be adenoviral vectors such as the human adenovirus of type 2 or 5.

A recombinant vector according to the invention may also be a retroviral vector or an adeno-associated vector (AAV). Such adeno-associated vectors are for example described by Flotte et al., Am. J. Respir., Cell Mol. Biol. (1992) 7: 349-356; Samulski et al., J. Virol. (1989) 63: 3822-3828; or McLaughlin et al., Am. J. Hum. Genet. (1996) 59: 561-569).

To allow the expression of a polynucleotide of interest under the control of a regulatory nucleic acid according to the invention, the polynucleotide construct comprising the regulatory sequence and the coding sequence must be introduced into a host cell. The introduction of such a polynucleotide construct according to the invention into a host cell may be carried out in vitro, according to the techniques well known to persons skilled in the art for transforming or transfecting cells, either in primary culture, or in the form of cell lines. It is also possible to carry out the introduction of the polynucleotides according to the invention in vivo or ex vivo, for the prevention or treatment of diseases linked to a deficiency in the transport of the ABCA7 protein.

To introduce the polynucleotides or the vectors into a host cell, persons skilled in the art can advantageously refer to various techniques, such as the technique for precipitation with calcium phosphate (Graham et al., Virology (1973) 52 456-457; Chen et al., Mol. Cell. Biol. (1987) 7: 2745-2752), DEAE Dextran (Gopal et al., Mol. Cell. Biol., (1985) 5: 1188-1190), electroporation (Tur-Kaspa et al., Mol. Cel. Biol, (1986) 6: 716-718.; Potter et al., Proc. Natl. Acad. Sci. USA (1984), 81(22), 7161-5), direct microinjection (Harland et al., J. Cell Biol (1985) 101: 1094-1095), liposomes charged with DNA (Nicolau et al., Methods Enzymol (1987) 149: 157-76; Fraley et al., Proc. Natl. Acad. Sci. USA (1979) 76: 3348-3352).

Once the polynucleotide has been introduced into the host cell, it may be stably integrated into the genome of the cell. The integration may be achieved at a precise site of the genome, by homologous recombination, or it may be randomly integrated. In some embodiments, the polynucleotide may be stably maintained in the host cell in the form of an episome fragment, the episome comprising sequences allowing the retention and the replication of the latter, either independently, or in a synchronized manner with the cell cycle.

According to a specific embodiment, a method of introducing a polynucleotide according to the invention into a host cell, in particular a host cell obtained from a mammal, in vivo, comprises a step during which a preparation comprising a pharmaceutically compatible vector and a “naked” polynucleotide according to the invention, placed under the control of appropriate regulatory sequences, is introduced by local injection at the level of the chosen tissue, for example a smooth muscle tissue, the “naked” polynucleotide being absorbed by the cells of this tissue.

Compositions for use in vitro and in vivo comprising “naked” polynucleotides are for example described in PCT Application No. WO 95/11307 as well as in the articles by Tacson et al. (Nature Med. (1996) 2(8), 888-892) and by Huygen et al., (Nat. Med. (1996) 2(8), 893-898).

According to a specific embodiment of the invention, a composition is provided for the in vivo production of a protein of interest. This composition comprises a polynucleotide encoding the polypeptide of interest placed under the control of a regulatory sequence according to the invention, in solution in a physiologically acceptable vector.

The quantity of vector which is injected into the host organism chosen varies according to the site of injection. As a guide, there may be injected between about 0.1 and about 100 μg of regulatory sequence/coding sequence polynucleotide construct into the body of an animal.

When the regulatory nucleic acid according to the invention is located on the polynucleotide construct (or vector), in such a manner as to control the transcription of a sequence comprising an open reading frame encoding the ABCA7 protein, the vector is preferably injected into the body of a patient likely to develop a disease linked to a deficiency in the ABCA7 protein.

Consequently, the invention also relates to a pharmaceutical composition intended for the prevention of or treatment of subjects affected by an ABCA7 protein dysfunction, comprising a regulatory nucleic acid according to the invention and a polynucleotide of interest encoding the ABCA7 protein, in combination with one or more physiologically compatible excipients.

Advantageously, such a composition will comprise the regulatory nucleic acid defined by one of the sequences SEQ ID No. 1 or 2, and SEQ ID No. 4 or 5, or a biologically active fragment of this regulatory nucleic acid.

The subject of the invention is, in addition, a pharmaceutical composition intended for the prevention of or treatment of subjects affected by a dysfunction in the metabolism of lipids, comprising a recombinant vector as defined above, in combination with one or more physiologically compatible excipients.

The subject of the invention is also a pharmaceutical composition intended for the prevention of or treatment of subjects affected by a dysfunction in the processes involving the immune system and inflammation, comprising a recombinant vector as defined above, in combination with one or more physiologically compatible excipients.

The invention also relates to the use of a polynucleotide construct in accordance with the invention and comprising a nucleic acid for regulating the ABCA7 gene as well as a sequence encoding the ABCA7 protein, for the manufacture of a medicament intended for the prevention of or treatment of subjects affected by a dysfunction in the metabolism of lipids or by a problem of immunological origin or of inflammatory origin.

The invention also relates to the use of a recombinant vector according to the invention, comprising, in addition to a regulatory nucleic acid of the invention, a nucleic acid encoding the ABCA7 protein, for the manufacture of a medicament intended for the prevention of or treatment of subjects affected by a dysfunction in the processes involving the immune system and inflammation.

Vectors Useful in Methods of Somatic Gene Therapy and Compositions Containing Such Vectors

The present invention also relates to a new therapeutic approach for the treatment and/or prevention of pathologies linked to the metabolism of lipids as well as for the treatment and/or prevention of pathologies linked to the dysfunction in the mechanisms of lymphocyte mediation of inflammation. It provides an advantageous solution to the disadvantages of the prior art, by demonstrating the possibility of treating pathologies, in particular pathologies linked to a dysfunction in the metabolism of lipids in myelo-lymphatic tissues, by gene therapy, by the transfer and the expression in vivo of a polynucleotide construct comprising, in addition to a regulatory nucleic acid according to the invention, a sequence encoding an ABCA7 protein which is highly presumed to be involved in the transport and/or metabolism of lipids. The invention thus offers a simple means allowing a specific and effective treatment of subjects affected by a dysfunction in the processes involving the immune system and inflammation.

Gene therapy consists in correcting a deficiency or an abnormality (mutation, aberrant expression and the like) or in bringing about the expression of a protein of therapeutic interest by introducing genetic information into the affected cell or organ. This genetic information may be introduced either ex vivo into a cell extracted from the organ, the modified cell then being reintroduced into the body, or directly in vivo into the appropriate tissue. In this second case, various techniques exist, among which various transfection techniques involving complexes of DNA and DEAE-dextran (Pagano et al., J.Virol. 1(1967) 891), of DNA and nuclear proteins (Kaneda et al., Science 243 (1989) 375), of DNA and lipids (Felgner et al., PNAS 84 (1987) 7413), the use of liposomes (Fraley et al., J.Biol.Chem. 255 (1980) 10431), and the like. More recently, the use of viruses as vectors for the transfer of genes has appeared as a promising alternative to these physical transfection techniques. In this regard, various viruses have been tested for their capacity to infect certain cell populations. In particular, the retroviruses (RSV, HMS, MMS, and the like), the HSV virus, the adeno-associated viruses and the adenoviruses.

The present invention therefore also relates to a new therapeutic approach for the treatment of pathologies linked to the transport of lipids, consisting in transferring and expressing in vivo genes encoding ABCA7 placed under the control of a regulatory acid according to the invention. It is particularly advantageous to construct recombinant viruses containing a DNA sequence comprising a regulatory nucleic acid according to the invention and a sequence encoding an ABCA7 protein involved in the metabolism of lipids, to administer these recombinant viruses in vivo, and that this administration allows a stable and effective expression of a biologically active ABCA7 protein in vivo and with no cytopathological effect.

The adenoviruses constitute particularly efficient vectors for the transfer and expression of the ABCA7 gene. In particular, the use of recombinant adenoviruses as vectors makes it possible to obtain sufficiently high levels of expression of the gene of interest to produce the desired therapeutic effect. Other viral vectors, such as retroviruses or adeno-associated viruses (AAV), allowing a stable expression of the gene are also claimed.

The present invention thus offers a new approach for the treatment and prevention of pathologies linked to dysfunctions in the metabolism of lipids and in the signaling pathways for inflammation by the lymphocytes.

The subject of the invention is therefore also a defective recombinant virus comprising a regulatory nucleic acid according to the invention and a nucleic sequence encoding an ABCA7 protein involved in the metabolism of lipids or in processes involving the immune system and inflammation.

The invention also relates to the use of such a defective recombinant virus for the preparation of a pharmaceutical composition intended for the treatment and/or prevention of dysfunctions in the signaling of inflammation by the lymphocytes.

The present invention also relates to the use of cells genetically modified ex vivo with a virus as described above, or of cells producing such as viruses, implanted in the body, allowing a prolonged and effective expression in vivo of a biologically active ABCA7 protein.

The present invention shows that it is possible to incorporate a DNA sequence encoding ABCA7 under the control of a regulatory nucleic acid as defined above into a viral vector, and that these vectors make it possible to effectively express a biologically active mature form. More particularly, the invention shows that the in vivo expression of ABCA7 may be obtained by direct administration of an adenovirus or by implantation of a producing cell or of a cell genetically modified by an adenovirus or by a retrovirus incorporating such a DNA.

The present invention is particularly advantageous because it makes it possible to induce a controlled expression, and with no harmful effect, of ABCA7 in organs which are not normally involved in the expression of this protein. In particular, a significant release of the ABCA7 protein is obtained by implantation of cells producing vectors of the invention, or infected ex vivo with vectors of the invention.

The mediator activity in the metabolism of lipids produced in the context of the present invention may be of the human or animal ABCA7 type. The nucleic sequence used in the context of the present invention may be a cDNA, a genomic DNA (gDNA), an RNA (in the case of retroviruses) or a hybrid construct consisting, for example, of a cDNA into which one or more introns would be inserted. It may also involve synthetic or semisynthetic sequences. In a particularly advantageous manner, a cDNA or a gDNA is used. In particular, the use of a gDNA allows a better expression in human cells. To allow their incorporation into a viral vector according to the invention, these sequences are advantageously modified, for example by site-directed mutagenesis, in particular for the insertion of appropriate restriction sites. The sequences described in the prior art are indeed not constructed for use according to the invention, and prior adaptations may prove necessary, in order to obtain substantial expressions. In the context of the present invention, the use of a nucleic sequence encoding a human ABCA7 protein is preferred. Moreover, it is also possible to use a construct encoding a derivative of these ABCA7 proteins. A derivative of these ABCA7 proteins comprises, for example, any sequence obtained by mutation, deletion and/or addition relative to the native sequence, and encoding a product retaining the activity of mediator of the metabolism of lipids. These modifications may be made by techniques F known to a person skilled in the art (see general molecular biological techniques below). The biological activity of the derivatives thus obtained can then be easily determined, as indicated in particular in the examples describing the measurement of the efflux of lipids from cells. The derivatives for the purposes of the invention may also be obtained by hybridization from nucleic acid libraries, using as probe the native sequence or a fragment thereof.

These derivatives are in particular molecules having a higher affinity for their binding sites, molecules exhibiting greater resistance to proteases, molecules having a higher therapeutic efficacy or fewer side effects, or optionally having new biological properties. The derivatives also include the modified DNA sequences allowing improved expression in vivo.

In a first embodiment, the present invention relates to a defective recombinant virus comprising a regulatory nucleic acid according to the invention and a cDNA sequence encoding an ABCA7 protein involved in the transport and metabolism of cholesterol. In another preferred embodiment of the invention, the DNA sequence is a gDNA sequence. The cDNA sequence encoding the ABCA7 protein, and which can be used in a vector according to the invention, is advantageously the sequence SEQ ID No. 8.

The vectors of the invention may be prepared from various types of viruses. Preferably, vectors derived from adenoviruses, adeno-associated viruses (AAV), herpesviruses (HSV) or retroviruses are used. It is most particularly advantageous to use an adenovirus, for direct administration or for the ex vivo modification of cells intended to be implanted, or a retrovirus, for the implantation of producing cells.

The viruses according to the invention are defective, that is to say that they are incapable of autonomously replicating in the target cell. Generally, the genome of the defective viruses used in the context of the present invention therefore lacks at least the sequences necessary for the replication of said virus in the infected cell. These regions may be either eliminated (completely or partially), or made nonfunctional, or substituted with other sequences and in particular with the nucleic sequence encoding the ABCA7 protein. Preferably, the defective virus retains, nevertheless, the sequences of its genome which are necessary for the encapsidation of the viral particles.

As regards more particularly adenoviruses, various serotypes, whose structure and properties vary somewhat, have been characterized. Among these serotypes, human adenoviruses of type 2 or 5 (Ad 2 or Ad 5) or adenoviruses of animal origin (see Application WO 94/26914) are preferably used in the context of the present invention. Among the adenoviruses of animal origin which can be used in the context of the present invention, there may be mentioned adenoviruses of canine, bovine, murine (example: Mav1, Beard et al., Virology 75 (1990) 81), ovine, porcine, avian or simian (example: SAV) origin. Preferably, the adenovirus of animal origin is a canine adenovirus, more preferably a CAV2 adenovirus [Manhattan or A26/61 strain (ATCC VR-800) for example]. Preferably, adenoviruses of human or canine or mixed origin are used in the context of the invention. Preferably, the defective adenoviruses of the invention comprise the ITRs, a sequence allowing the encapsidation and the sequence encoding the ABCA7 protein placed under the control of a nucleic acid according to the invention. Advantageously, in the genome of the adenoviruses of the invention, the E1 region at least is made nonfunctional. Still more preferably, in the genome of the adenoviruses of the invention, the E1 gene and at least one of the E2, E4 and L1-L5 genes are nonfunctional. The viral gene considered may be made nonfunctional by any technique known to a person skilled in the art, and in particular by total suppression, by substitution, by partial deletion or by addition of one or more bases in the gene(s) considered. Such modifications may be obtained in vitro (on the isolated DNA) or in situ, for example, by means of genetic engineering techniques, or by treatment by means of mutagenic agents. Other regions may also be modified, and in particular the E3 (WO95/02697), E2 (WO94/28938), E4 (WO94/28152, WO94/12649, WO95/02697) and L5 (WO95/02697) region. According to a preferred embodiment, the adenovirus according to the invention comprises a deletion in the E1 and E4 regions and the sequence encoding ABCA7 is inserted at the level of the inactivated E1 region. According to another preferred embodiment, it comprises a deletion in the E1 region at the level of which the E4 region and the sequence encoding ABCA7 (French Patent Application FR94 13355) are inserted.

The defective recombinant adenoviruses according to the invention may be prepared by any technique known to persons skilled in the art (Levrero et al., Gene (1991) 101: 195, EP 185 573; Graham, EMBO J. (1984) 3: 2917). In particular, they may be prepared by homologous recombination between an adenovirus and a plasmid carrying, inter alia, the DNA sequence encoding the ABCA7 protein. The homologous recombination occurs after cotransfection of said adenoviruses and plasmid into an appropriate cell line. The cell line used must preferably (i) be transformable by said elements, and (ii), contain the sequences capable of complementing the part of the defective adenovirus genome, preferably in integrated form in order to avoid the risks of recombination. By way of example of a line, there may be mentioned the human embryonic kidney line 293 (Graham et al., J. Gen. Virol. (1977) 36: 59) which contains in particular, integrated into its genome, the left part of the genome of an Ad5 adenovirus (12%) or lines capable of complementing the E1 and E4 functions as described in particular in Applications No. WO 94/26914 and WO95/02697.

Next, the adenoviruses which have multiplied are recovered and purified according to conventional molecular biological techniques, as illustrated in the examples.

As regards the adeno-associated viruses (AAV), they are DNA viruses of a relatively small size, which integrate into the genome of the cells which they infect, in a stable and site-specific manner. They are capable of infecting a broad spectrum of cells, without inducing any effect on cellular growth, morphology or differentiation. Moreover, they do not appear to be involved in pathologies in humans. The genome of AAVs has been cloned, sequenced and characterized. It comprises about 4700 bases, and contains at each end an inverted repeat region (ITR) of about 145 bases, serving as replication origin for the virus. The remainder of the genome is divided into 2 essential regions carrying the encapsidation functions: the left hand part of the genome, which contains the rep gene involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see in particular WO 91/18088; WO 93/09239; U.S. Pat. No. 4,797,368, U.S. Pat. No. 5,139,941, EP 488 528). These documents describe various constructs derived from AAVs, in which the rep and/or cap genes are deleted and replaced by a gene of interest, and their use for transferring in vitro (on cells in culture) or in vivo (directly into an organism) said gene of interest. However, none of these documents either describes or suggests the use of a recombinant AAV for the transfer and expression in vivo or ex vivo of an ABCA7 protein, or the advantages of such a transfer. The defective recombinant AAVs according to the invention may be prepared by cotransfection, into a cell line infected with a human helper virus (for example an adenovirus), of a plasmid containing the sequence encoding the ABCA7 protein bordered by two AAV inverted repeat regions (ITR), and of a plasmid carrying the AAV encapsidation genes (rep and cap genes). The recombinant AAVs produced are then purified by conventional techniques.

As regards the herpesviruses and the retroviruses, the construction of recombinant vectors has been widely described in the literature: see in particular Breakfield et al., (New Biologist 3 (1991) 203); EP 453242, EP 178220, Bernstein et al. (Genet. Eng. 7 (1985) 235); McCormick, (BioTechnology 3 (1985) 689), and the like.

In particular, the retroviruses are integrating viruses, infecting dividing cells. The genome of the retroviruses essentially comprises two LTRs, an encapsidation sequence and three coding regions (gag, pol and env). In the recombinant vectors derived from retroviruses, the gag, pol and env genes are generally deleted, completely or partially, and replaced with a heterologous nucleic acid sequence of interest. These vectors may be produced from various types of retroviruses such as in particular MoMuLV (“murine moloney leukemia virus”; also called MOMLV), MSV (“murine moloney sarcoma virus”), HaSV (“harvey sarcoma virus”); SNV (“spleen necrosis virus”); RSV (“rous sarcoma virus”) or Friend's virus.

To construct recombinant retroviruses containing a sequence encoding the ABCA7 protein placed under the control of a regulatory nucleic acid according to the invention, a plasmid containing in particular the LTRs, the encapsidation sequence and said coding sequence is generally constructed, and then used to transfect a so-called encapsidation cell line, capable of providing in trans the retroviral functions deficient in the plasmid. Generally, the encapsidation lines are therefore capable of expressing the gag, pol and env genes. Such encapsidation lines have been described in the prior art, and in particular the PA317 line (U.S. Pat. No. 4,861,719), the PsiCRIP line (WO 90/02806) and the GP+envAm-12 line (WO 89/07150). Moreover, the recombinant retroviruses may contain modifications at the level of the LTRs in order to suppress the transcriptional activity, as well as extended encapsidation sequences, containing a portion of the gag gene (Bender et al., J. Virol. 61 (1987) 1639). The recombinant retroviruses produced are then purified by conventional techniques.

To carry out the present invention, it is most particularly advantageous to use a defective recombinant adenovirus. The results given below indeed demonstrate the particularly advantageous properties of adenoviruses for the in vivo expression of a protein having a lipid metabolism mediator activity. The adenoviral vectors according to the invention are particularly advantageous for a direct administration in vivo of a purified suspension, or for the ex vivo transformation of cells, in particular autologous cells, in view of their implantation. Furthermore, the adenoviral vectors according to the invention exhibit, in addition, considerable advantages, such as in particular their very high infection efficiency, which makes it possible to carry out infections using small volumes of viral suspension.

According to another particularly advantageous embodiment of the invention, a line producing retroviral vectors containing a regulatory nucleic acid according to the invention and the sequence encoding the ABCA7 protein is used for implantation in vivo. The lines which can be used to this end are in particular the PA317 (U.S. Pat. No. 4,861,719), PsiCrip (WO 90/02806) and GP+envAm-12 (U.S. Pat. No. 5,278,056) cells modified so as to allow the production of a retrovirus containing a nucleic sequence encoding an ABCA7 protein according to the invention. For example, totipotent stem cells, precursors of blood cell lines, may be collected and isolated from the subject. These cells, when cultured, may then be transfected with the retroviral vector containing the sequence encoding the ABCA7 protein under the control of its own promoter. These cells are then reintroduced into the subject. The differentiation of these cells will be responsible for cells of the hematopoietic tissue expressing the ABCA7 protein, in particular T lymphocytes which participate in the signaling of inflammation.

Advantageously, in the vectors of the invention, the sequence encoding the ABCA7 protein is placed under the control of a regulatory acid according to the invention comprising the regulatory elements allowing its expression in the infected cells, and most particularly the regulatory elements of the NFkappaB, CEBP and GFI1 type.

As indicated above, the present invention also relates to any use of a virus as described above for the preparation of a pharmaceutical composition intended for the treatment and/or prevention of pathologies linked to the metabolism of lipids or to the dysfunction linked to the processes involving the immune system and inflammation.

The present invention also relates to a pharmaceutical composition comprising one or more defective recombinant viruses as described above. These pharmaceutical compositions may be formulated for administration by the topical, oral, parenteral, intranasal, intravenous, intramuscular, subcutaneous, intraocular or transdermal route and the like. Preferably, the pharmaceutical compositions of the invention contain a pharmaceutically acceptable vehicle for an injectable formulation, in particular for an intravenous injection, such as for example into the patient's portal vein. They may relate in particular to isotonic sterile solutions or dry, in particular freeze-dried, compositions which, upon addition depending on the case of sterilized water or physiological saline, allow the preparation of injectable solutions. Direct injection into the patient's portal vein is advantageous because it makes it possible to target the infection at the level of the liver and thus to concentrate the therapeutic effect at the level of this organ.

The doses of defective recombinant virus used for the injection may be adjusted as a function of various parameters, and in particular as a function of the viral vector, of the mode of administration used, of the relevant pathology or of the desired duration of treatment. In general, the recombinant adenoviruses according to the invention are formulated and administered in the form of doses of between 104 and 1014 pfu/ml, and preferably 106 to 1010 pfu/ml. The term pfu (“plaque forming unit”) corresponds to the infectivity of a virus solution, and is determined by infecting an appropriate cell culture and measuring, generally after 48 hours, the number of plaques of infected cells. The techniques for determining the pfu titer of a viral solution are well documented in the literature.

As regards retroviruses, the compositions according to the invention may directly contain the producing cells, with a view to their implantation.

In this regard, another subject of the invention relates to any mammalian cell infected with one or more defective recombinant viruses as described above. More particularly, the invention relates to any population of human cells infected with these viruses. These may be in particular cells of blood origin (totipotent stem cells or precursors), fibroblasts, myoblasts, hepatocytes, keratinocytes, smooth muscle and endothelial cells, glial cells and the like.

The cells according to the invention may be derived from primary cultures. These may be collected by any technique known to persons skilled in the art and then cultured under conditions allowing their proliferation. As regards more particularly fibroblasts, these may be easily obtained from biopsies, for example according to the technique described by Ham (Methods Cell Biol (1980) 21a: 255). These cells may be used directly for infection with the viruses, or stored, for example by freezing, for the establishment of autologous libraries, in view of a subsequent use. The cells according to the invention may be secondary cultures, obtained for example from preestablished libraries (see for example EP 228458, EP 289034, EP 400047, EP 456640).

The cells in culture are then infected with the recombinant viruses, in order to confer on them the capacity to produce a biologically active ABCA7 protein. The infection is carried out in vitro according to techniques known to persons skilled in the art. In particular, depending on the type of cells used and the desired number of copies of virus per cell, persons skilled in the art can adjust the multiplicity of infections and optionally the number of infectious cycles produced. It is clearly understood that these steps must be carried out under appropriate conditions of sterility when the cells are intended for administration in vivo. The doses of recombinant virus used for the infection of the cells may be adjusted by persons skilled in the art according to the desired aim. The conditions described above for the administration in vivo may be applied to the infection in vitro. For the infection with retroviruses, it is also possible to coculture the cells which it is desired to infect with cells producing the recombinant retroviruses according to the invention. This makes it possible to dispense with the purification of the retroviruses.

Another subject of the invention relates to an implant comprising mammalian cells infected with one or more defective recombinant viruses as described above or cells producing recombinant viruses, and an extracellular matrix. Preferably, the implants according to the invention comprise 10⁵ to 10¹⁰ cells. More preferably, they comprise 10⁶ to 10⁸ cells.

More particularly, in the implants of the invention, the extracellular matrix comprises a gelling compound and optionally a support allowing the anchorage of the cells.

For the preparation of the implants according to the invention, various types of gelling agents may be used. The gelling agents are used for the inclusion of the cells in a matrix having the constitution of a gel, and for promoting the anchorage of the cells on the support, where appropriate. Various cell adhesion agents can therefore be used as gelling agents, such as in particular collagen, gelatin, glycosaminoglycans, fibronectin, lectins and the like. Preferably, collagen is used in the context of the present invention. This may be collagen of human, bovine or murine origin. More preferably, type I collagen is used.

As indicated above, the compositions according to the invention advantageously comprise a support allowing the anchorage of the cells. The term anchorage designates any form of biological and/or chemical and/or physical interaction causing the adhesion and/or the attachment of the cells to the support. Moreover, the cells may either cover the support used, or penetrate inside this support, or both. It is preferable to use in the context of the invention a solid, nontoxic and/or biocompatible support. In particular, it is possible to use polytetrafluoroethylene (PTFE) fibers or a support of biological origin.

The present invention thus offers a very effective means for the treatment or prevention of pathologies linked to the transport of cholesterol, in particular obesity, hypertriglyceridemia, or, in the field of cardiovascular conditions, myocardial infarction, angina, sudden death, cardiac decompensation and cerebrovascular accidents.

In addition, this treatment may be applied to both humans and any animals such as ovines, bovines, domestic animals (dogs, cats and the like), horses, fish and the like.

Recombinant Host Cells

The invention also relates to a recombinant host cell comprising any one of the nucleic acids of the invention having the sequence SEQ ID No. 1 to SEQ ID No. 6, and more particularly a nucleic acid having the sequence SEQ ID NO 1 to SEQ ID No. 3.

According to another aspect, the invention also relates to a recombinant host cell comprising a recombinant vector as described above.

The preferred host cells according to the invention are for example the following:

a) prokaryotic host cells: strains of Escherichia coli (strain DH5-), of Bacillus subtilis, of Salmonella typhimurium, or strains of species such as Pseudomonas, Streptomyces and Staphylococus;

b) eukaryotic host cells: HeLa cells (ATCC No. CCL2), Cv 1 cells (ATCC No. CCL70), COS cells (ATCC No. CRL 1650), Sf-9 cells (ATCC No. CRL 1711), CHO cells (ATCC No. CCL-61) or 3T3 cells (ATCC No. CRL-6361), or cells of the Hepa 1-6 line referenced at the American Type Culture Collection (ATCC, Rockville, Md., United States of America).

c) primary culture cells obtained from an individual in whom the expression of a nucleic acid of interest, placed under the control of a regulatory nucleic acid according to the invention, is sought.

d) cells multiplying indefinitely (cell lines) obtained from the primary culture cells of c) above, according to techniques well known to persons skilled in the art.

Methods of Screening

Method of Screening in vitro

The invention provides methods for treating a subject suffering from a pathology linked to the level of expression of the ABCA7 protein. In particular, such a method of treatment consists in administering to the subject a compound modulating the expression of the ABCA7 gene, which may be identified by various methods of screening in vitro as defined below.

A first method consists in identifying compounds modulating the expression of the ABCA7 gene. According to such a method, cells expressing the ABCA7 gene are incubated with a candidate substance or molecule to be tested and the level of expression of the messenger RNA for ABCA7 or the level of production of the ABCA7 protein is then determined.

The levels of messenger RNA for ABCA7 may be determined by gel hybridization of the Northern type, well known to persons skilled in the art. The levels of messenger RNA for ABCA7 may also be determined by methods using PCR or the technique described by WEBB et al. (Journal of Biomolecular Screening (1996), vol. 1: 119).

The levels of production of the ABCA7 protein may be determined by immunoprecipitation or immunochemistry using an antibody which specifically recognizes the ABCA7 protein.

According to another method of screening a candidate molecule or substance modulating the activity of a regulatory nucleic acid according to the invention, a nucleotide construct as defined above, comprising a regulatory nucleic acid according to the invention as well as a reporter polynucleotide placed under the control of the regulatory nucleic acid, is used, said regulatory nucleic acid comprising at least one core promoter and at least one element for regulating one of the sequences SEQ ID No. 1 to SEQ ID No. 3. The reporter polynucleotide may be a gene encoding a detectable protein, such as a gene encoding a luciferase.

According to such a screening method, the cells are transfected with the polynucleotide construct containing the regulatory nucleic acid according to the invention and the reporter polynucleotide, in a stable and transient manner.

The transformed cells are then incubated in the presence or in the absence of the candidate molecule or substance to be tested for a sufficient time, and then the level of expression of the reporter gene is determined. The compounds which induce a statistically significant change in the expression of the reporter gene (either an increase, or on the contrary a decrease in the expression of the reporter gene) are then identified and, where appropriate, selected.

Thus, the subject of the invention is also a method for the in vitro screening of a molecule or substance modulating the activity of a regulatory nucleic acid according to the invention, in particular modulating the transcription of the constitutive reporter polynucleotide of a polynucleotide construct according to the invention, characterized in that it comprises the steps consisting in:

a) culturing a recombinant host cell comprising a polynucleotide of interest placed under the control of a regulatory nucleic acid according to the invention;

b) incubating the recombinant host cell with the substance or molecule to be tested;

c) detecting the expression of the polynucleotide of interest;

d) comparing the results obtained in step c) with the results obtained when the recombinant host cell is cultured in the absence of the candidate molecule or substance to be tested.

The invention also relates to a kit or box for the in vitro screening of a candidate molecule or substance capable of modulating the activity of a regulatory nucleic acid according to the invention, comprising:

a) a host cell transformed with a polynucleotide construct as defined above, comprising a reporter polynucleotide of interest placed under the control of a regulatory nucleic acid according to the invention; and

b) where appropriate, means for detecting the expression of the reporter polynucleotide of interest.

Preferably, the reporter polynucleotide of interest is the luciferase coding sequence. In this case, the regulatory nucleic acid according to the invention is inserted into a vector, upstream of the sequence encoding luciferase. This may be for example the vector pGL3-basic (pGL3-b) marketed by the company Promega (Madison, Wis., United States).

In this case, the recombinant vector comprising the luciferase coding sequence placed under the control of a regulatory nucleic acid according to the invention is transfected into the recombinant host cells whose luciferase activity is then determined after culturing in the presence or in the absence of the candidate substance or molecule to be tested.

It is possible in this case to use as controls pGL3-b vectors containing either the cytomegalovirus (CMV) promoter, the ApoAl promoter or no promoter. To test for the luciferase activity, the transfected cells are washed with a PBS buffer and lyzed with 500 μl of lysis buffer (50 mM TRIS, 150 mM NaCl, 0.02% sodium azide, 1% of NP-40, 100 μg/ml of AEBSF and 5 μg/ml of leupeptin).

50 μl of the cell lysate obtained are then added to 100 μl of the luciferase substrate (Promega) and the measurements of activity are carried out on a spectrophotometric microplate reader, 5 minutes after the addition of the cell lysate.

The data are expressed as relative units of luciferase activity. The polynucleotide constructs producing high levels of luciferase activity in the transfected cells are those which contain a regulatory nucleic acid according to the invention contained in the sequence SEQ ID No. 1 which is capable of stimulating transcription.

For the measurements of the levels of expression of messenger RNA in a screening method according to the invention, probes specific for the messenger RNA for the reporter polynucleotide of interest are first of all prepared, for example with the aid of the multiprime labeling kit (marketed by the company Amersham Life Sciences, Cleveland, Ohio, United States).

Method of Screening in vivo

According to another aspect of the invention, compositions modulating the activity of a regulatory nucleic acid according to the invention may be identified in vivo, in nonhuman transgenic animals.

According to such a method, a nonhuman transgenic animal, for example a mouse, is treated with a candidate molecule or substance to be tested, for example a candidate substance or molecule previously selected by an in vitro screening method as defined above.

After a defined period, the level of activity of the regulatory nucleic acid according to the invention is determined and compared with the activity of an identical nonhuman transgenic animal, for example an identical transgenic mouse, which has not received the candidate molecule or substance.

The activity of the regulatory nucleic acid according to the invention which is functional in the transgenic animal may be determined by various methods, for example the measurement of the levels of messenger RNA corresponding to the reporter polynucleotides of interest placed under the control of said regulatory nucleic acid by Northern-type hybridization, or by in situ hybridization or by noninvasive biophotonic imaging (Xenogen Corporation).

Alternatively, the activity of the regulatory nucleic acid according to the invention may be determined by measuring the levels of expression of the protein encoded by the reporter polynucleotides of interest, for example by immunohistochemistry, in the case where the reporter polynucleotide of interest comprises an open reading frame encoding a protein detectable by such a technique.

To carry out an in vivo method of screening a candidate substance or molecule modulating the activity of a regulatory nucleic acid according to the invention, nonhuman mammals such as mice, rats, guinea pigs or rabbits whose genome has been modified by the insertion of a polynucleotide construct comprising a reporter polynucleotide of interest placed under the control of a regulatory nucleic acid according to the invention, will be preferred.

The transgenic animals according to the invention comprise the transgene, that is to say the abovementioned polynucleotide construct, in a plurality of their somatic and/or germ cells.

The construction of transgenic animals according to the invention may be carried out according to conventional techniques well known to persons skilled in the art. Persons skilled in the art will in particular be able to refer to the production of transgenic animals, and particularly to the production of transgenic mice, as described in U.S. Pat. No. 4,873,191 (granted on Oct. 10, 1989), U.S. Pat. No. 5,464,764 (granted on Nov. 7, 1995) and U.S. Pat. No. 5,789,215 (granted on Aug. 4, 1998), the content of these documents being incorporated herein by reference.

In brief, a polynucleotide construct comprising a regulatory nucleic acid according to the invention and a reporter polynucleotide of interest placed under the control of the latter is inserted into an ES-type stem cell line. The insertion of the polynucleotide construct is preferably carried out by electroporation, as described by Thomas et al. (1987, Cell, 51:503-512).

The cells which have been subjected to the electroporation step are then screened for the presence of the polynucleotide construct (for example by selection with the aid of markers, or by PCR or by Southern-type analysis of DNA on an electrophoresis gel) in order to select the positive cells which have integrated the exogenous polynucleotide construct into their genome, where appropriate following a homologous recombination event. Such a technique is for example described by Mansour et al. (Nature (1988) 336: 348-352).

Next, the positively selected cells are isolated, cloned and injected into 3.5-day old mouse blastocysts, as described by Bradley (1987, Production and Analysis of Chimaeric mice. In: E. J. Robertson (Ed., Teratocarcinomas and embryonic stem cells: A practical approach. IRL press, Oxford, page 113). The blastocysts are then introduced into a female host animal and the development of the embryo is continued to term.

Alternatively, positively selected ES-type cells are brought into contact with 2.5-day old embryos at an 8-16 cell stage (morulae) as described by Wood et al. (1993, Proc. Natl. Acad. Sci. USA, vol.90: 4582-4585) or by Nagy et al. (1993, Proc. Natl. Acad. Sci. USA, vol. 90: 8424-8428), the ES cells being internalized so as to extensively colonize the blastocyst, including the cells producing the germ line.

The progeny is then tested in order to determine those which have integrated the polynucleotide construct (the transgene).

The subject of the invention is therefore also a nonhuman transgenic animal whose somatic and/or germ cells have been transformed with a nucleic acid or a polynucleotide construct according to the invention.

The invention also relates to recombinant host cells obtained from a transgenic animal as described above.

Recombinant cell lines obtained from a transgenic animal according to the invention may be established in a long-term culture from any tissue of such a transgenic animal, for example by transfection of the primary cell cultures with vectors expressing oncogenes such as the SV40 large T antigen, as described for example by Chou (1989, Mol. Endocrinol. 3: 1511-1514) and Schay et al. (1991, Biochem. Biophys. Acta, 1072: 1-7).

The invention also relates to a method for the in vivo screening of a candidate molecule or substance modulating the activity of a regulatory nucleic acid according to the invention, comprising the steps of:

a) administering the candidate substance or molecule to a transgenic animal as defined above;

b) detecting the level of expression of a reporter polynucleotide of interest placed under the control of the regulatory nucleic acid;

c) comparing the results obtained in b) with the results obtained with a transgenic animal which has not received the candidate substance or molecule.

The invention also relates to a kit or box for the in vivo screening of a candidate molecule or substance modulating the activity of a regulatory nucleic acid according to the invention, comprising:

a) a transgenic animal as defined above;

b) where appropriate, the means for detecting the level of expression of the reporter polynucleotide of interest.

Pharmaceutical Compounds and Compositions

The invention also relates to pharmaceutical compositions intended for the prevention or treatment of a deficiency in the metabolism of lipids, or of a dysfunction in the processes involving the immune system and inflammation.

First, the subject of the invention is also a candidate substance or molecule modulating the activity of a regulatory nucleic acid according to the invention.

The invention also relates to a candidate substance or molecule characterized in that it increases the activity of a regulatory nucleic acid according to the invention, and most particularly of a regulatory nucleic acid comprising the sequence SEQ ID No. 1, 2, 4 or 5.

Preferably, such a substance or molecule capable of modulating the activity of a regulatory nucleic acid according to the invention was selected according to one of the in vitro or in vivo screening methods defined above.

Thus, a subject impaired in the metabolism of lipids or in immunity signaling is treated by the administration to this subject of an effective quantity of a compound modulating the activity of a regulatory nucleic acid according to the invention.

Thus, a patient having a weak ABCA7 promoter activity may be treated with an abovementioned molecule or substance in order to increase the activity of the ABCA7 promoter.

Alternatively, a patient having an abnormally high ABCA7 promoter activity may be treated with a compound capable of reducing or blocking the activity of the ABCA7 promoter.

Such a compound may be a compound which modulates the interaction of at least one transcription factor with the ABCA7 promoter or a regulatory element of a regulatory nucleic acid according to the invention.

For example, the compound may inhibit the interaction of one of the transcription factors listed in Table 1 with a regulatory nucleic acid according to the invention.

The compound may also be a compound which modulates the activity of a transcription factor which binds to the ABCA7 promoter or a regulatory element present on the latter.

A compound of therapeutic interest according to the invention may also be a compound which modulates the interaction of a first transcription factor with a second transcription factor.

As detailed in the analysis of the various transcription factors capable of binding to the sequence SEQ ID No. 1, 2, 4 or 5, some transcription factors are active only if they are combined with another transcription factor.

A compound of therapeutic interest according to the invention is preferably chosen from nucleic acids, peptides and small molecules. For example, such a compound may be an antisense nucleic acid which specifically binds to one region of the ABCA7 promoter or to a regulatory element of a nucleic acid for regulating ABCA7 and inhibiting or reducing the activity of the promoter.

This compound of therapeutic interest may also be an antisense nucleic acid which interacts specifically with a gene encoding a transcription factor modulating the activity of the ABCA7 promoter, in a manner such that the interaction of the antisense nucleic acid with the gene encoding the transcription factor binding to the ABCA7 promoter reduces the production of this transcription factor, resulting in an increase or a decrease in the activity of the ABCA7 promoter, depending on whether the transcription factor increases or on the contrary reduces the activity of the ABCA7 promoter.

The toxicity and the therapeutic efficacy of the therapeutic compounds according to the invention may be determined according to standard pharmaceutical protocols in cells in culture or in experimental animals, for example in order to determine the lethal dose LD50 (that is to say the dose which is lethal for 50% of the population tested) as well as the effective dose ED50 (that is to say the dose which is therapeutically effective in 50% of the population tested).

For all the compounds of therapeutic interest according to the invention, the therapeutically effective dose may be initially estimated from tests carried out in cell cultures in vitro.

The subject of the invention is also pharmaceutical compositions comprising a therapeutically effective quantity of a substance or molecule of therapeutic interest according to the invention.

Such pharmaceutical compositions may be formulated in a conventional manner using one or more physiologically acceptable vectors or excipients.

Thus, the compounds of therapeutic interest according to the invention, as well as their physiologically acceptable salts and solvates, may be formulated for administration by injection, inhalation or by oral, buccal, parenteral or rectal administration.

Techniques for the preparation of pharmaceutical compositions according to the invention can be easily found by persons skilled in the art, for example in the manual Remmington's Pharmaceutical Sciences, Mead Publishing Co., Easton, Pa., United States.

For a systemic administration, injection will be preferred, including intramuscular, intravenous, intraperitoneal and subcutaneous injections. In this case, the pharmaceutical compositions according to the invention may be formulated in the form of liquid solutions, preferably in physiologically compatible solutions or buffers.

Method for the Detection of an Impairment in the Transcription of the Human ABCA7 Gene

The subject of the invention is in addition methods for determining if a subject is at risk of developing a pathology linked to a deficiency in the metabolism of lipids, or in the processes involving the immune system and inflammation.

Such methods comprise the detection, in cells of a biological sample obtained from the subject to be tested, of the presence or of the absence of a genetic impairment characterized by impairment of the expression of a gene whose expression is regulated by the ABCA7 promoter.

By way of illustration, such genetic impairments may be detected in order to determine the existence of a deletion of one or more nucleotides in the sequence of a nucleic acid for regulating ABCA7, of the addition of one or more nucleotides or of the substitution of one or more nucleotides in said sequence SEQ ID No. 1, 2, 3 or 6.

According to a specific embodiment of a method for the detection of an impairment of the transcription of the ABCA7 gene in a subject, the genetic impairment is identified according to a method comprising the sequencing of all or part of the sequence SEQ ID No. 1, or alternatively of all or part of at least the sequence SEQ ID No. 2.

Sequencing primers may be constructed so as to hybridize with a defined region of the sequence SEQ ID No. 1. Such sequencing primers are preferably constructed so as to amplify fragments of about 300 to about 500 nucleotides of the sequence SEQ ID No. 1 or of a complementary sequence.

The fragments amplified, for example by the PCR method, are then sequenced and the sequence obtained is compared with the reference sequence SEQ ID No. 1 in order to determine if one or more deletions, additions or substitutions of nucleotides are found in the sequence amplified from the DNA contained in the biological sample obtained from the subject tested.

The invention therefore also relates to a method of detecting an impairment of the transcription of the ABCA7 gene in a subject, comprising the following steps:

a) sequencing of a nucleic acid fragment amplifiable with the aid of at least one nucleotide primer hybridizing with the sequence SEQ ID No. 1 or SEQ ID No. 2, according to the invention;

b) aligning the sequence obtained in a) with the sequence SEQ ID No. 1 or SEQ ID No. 2;

c) determining the presence of one or more deletions, additions or substitutions of at least one nucleotide in the sequence of the nucleic acid fragment, relative to the reference sequence SEQ ID No. 1 or SEQ ID No. 2.

In addition, also forming part of the invention are oligonucleotide probes hybridizing with a region of the sequence SEQ ID No. 1 or of the sequence SEQ ID No. 2 in which an impairment in the sequence has been determined during the implementation of the method of detection described above.

Alternatively, also forming part of the invention are oligonucleotide probes hybridizing specifically with a corresponding region of the sequence SEQ ID No. 1 or of the sequence SEQ ID No. 2 for which one or more deletions, additions or substitutions of at least one nucleotide has been determined in a subject.

Such oligonucleotide probes constitute means of detecting impairments in the sequence for regulating the ABCA7 gene and therefore also means for detecting a predisposition to the development of a pathology linked to a deficiency in the metabolism of lipids or to dysfunction in the processes involving the immune system and inflammation.

The subject of the invention is therefore also a kit or box for the detection of an impairment of the transcription of the ABCA7 gene in a subject, comprising:

a) one or more primers hybridizing with a region of the sequence SEQ ID No. 1 or of the sequence SEQ ID No. 2;

b) where appropriate, the means necessary for carrying out an amplification reaction.

The subject of the invention is also a kit or box for the detection of an impairment of the transcription of the ABCA7 gene in a subject, comprising:

a) one or more oligonucleotide probes as defined above;

b) where appropriate, the reagents necessary for carrying out a hybridization reaction.

The nucleic acid fragments derived from any one of the nucleotide sequences SEQ ID No. 1-6 are therefore useful for the detection of the presence of at least one copy of a nucleotide sequence for regulating the ABCA7 gene or a fragment or a variant (containing a mutation or a polymorphism) thereof in a sample.

The nucleotide probes or primers according to the invention comprise at least 8 consecutive nucleotides of a nucleic acid chosen from the group consisting of the sequences SEQ ID NO 1-5, or of a nucleic acid having a complementary sequence.

Preferably, nucleotide probes or primers according to the invention will have a length of 10, 12,15, 18 or 20 to 25, 35,40, 50, 70, 80, 100, 200, 500, 1000, 1500 consecutive nucleotides of a nucleic acid according to the invention, in particular a nucleic acid having a nucleotide sequence chosen from the sequences SEQ ID NO. 1-5.

Alternatively, a nucleotide probe or primer according to the invention will consist of and/or comprise the fragments having a length of 12, 15, 18, 20, 25, 35, 40, 50, 100, 200, 500, 1000, 1500 consecutive nucleotides of a nucleic acid according to the invention, more particularly of a nucleic acid chosen from the sequences SEQ ID No. 1-5, or of a nucleic acid having a complementary sequence.

The definition of a nucleotide probe or primer according to the invention therefore covers oligonucleotides which hybridize, under the high stringency hybridization conditions defined above, with a nucleic acid chosen from the sequences SEQ ID NO 1-5, 6 or 8 or with a sequence complementary thereto.

Examples of primers and pairs of primers which make it possible to amplify various regions of the ABCA7 gene are represented below.

This includes for example the pair of primers represented by the primer having the sequence SEQ ID No. 9: AGCCAGCAACGCAATCCTCC and the primer having the sequence SEQ ID No. 10: CGCACCATGTCAATGAGCCC.

A nucleotide primer or probe according to the invention may be prepared by any suitable method well known to persons skilled in the art, including by cloning and action of restriction enzymes or by direct chemical synthesis according to techniques such as the phosphodiester method by Narang et al., (Methods Enzymol (1979) 68:90-98) or by Brown et al. (Methods Enzymol (1979) 68:109-151), the diethylphosphoramidite method by Beaucage et al. (Tetrahedron Lett (1980) 22: 1859-1862) or the technique on a solid support described in patent EP 0,707,592.

Each of the nucleic acids according to the invention, including the oligonucleotide probes and primers described above, may be labeled, if desired, by incorporating a marker which can be detected by spectroscopic, photochemical, biochemical, immunochemical or chemical means.

For example, such markers may consist of radioactive isotopes (32P, 33P, 3H, 35S), fluorescent molecules (5-bromodeoxyuridine, fluorescein, acetylaminofluorene, digoxigenin) or ligands such as biotin.

The labeling of the probes is preferably carried out by incorporating labeled molecules into the polynucleotides by primer extension, or alternatively by addition to the 5′ or 3′ ends or by “nick translation”.

Examples of nonradioactive labeling of nucleic acid fragments are described in particular in French patent No. 78 109 75 or in the articles by Urdea et al. (Nucleic Acid Res (1988) 11: 4937-4957) or Sanchez-pescador et al. (J. Clin Mircrobiol (1988) 26(10) 1934-1938).

Advantageously, the probes according to the invention may have structural characteristics of the type to allow amplification of the signal, such as the probes described by Urdea et al.(Mol. Cell. Biol., (1991) 6:716-718), or alternatively in European patent No. EP-0,225,807 (CHIRON).

The oligonucleotide probes according to the invention may be used in particular in Southern-type hybridizations with genomic DNA or alternatively Northern-type hybridizations with RNA.

The probes according to the invention may also be used for the detection of products of PCR amplification or alternatively for the detection of mismatches.

Nucleotide probes or primers according to the invention may be immobilized on a solid support. Such solid supports are well known to persons skilled in the art and comprise surfaces of wells of microtiter plates, polystyrene beds, magnetic beds, nitrocellulose bands or microparticles such as latex particles.

Consequently, the present invention also relates to a method of detecting the presence of a nucleic acid as described above in a sample, said method comprising the steps of:

1) bringing one or more nucleotide probes according to the invention into contact with the sample to be tested;

2) detecting the complex which may have formed between the probe(s) and the nucleic acid present in the sample.

According to a specific embodiment of the method of detection according to the invention, the oligonucleotide probe(s) are immobilized on a support.

According to another aspect, the oligonucleotide probes comprise a detectable marker.

The invention relates, in addition, to a box or kit for detecting the presence of a nucleic acid according to the invention in a sample, said box comprising:

a) one or more nucleotide probes as described above;

b) where appropriate, the reagents necessary for the hybridization reaction.

According to a first aspect, the detection box or kit is characterized in that the probe(s) are immobilized on a support.

According to a second aspect, the detection box or kit is characterized in that the oligonucleotide probes comprise a detectable marker.

According to a specific embodiment of the detection kit described above, such a kit will comprise a plurality of oligonucleotide probes in accordance with the invention which may be used to detect target sequences of interest or alternatively to detect mutations in the coding regions or the noncoding regions of the nucleic acids according to the invention, more particularly of the nucleic acids having the sequences SEQ ID NO 1-5, 6 and 8 or the nucleic acids having a complementary sequence.

Thus, the probes according to the invention, immobilized on a support, may be ordered into matrices such as “DNA chips”. Such ordered matrices have in particular been described in U.S. Pat. No. 5,143,854, in PCT applications No. WO 90/150 70 and 92/10092.

Support matrices on which oligonucleotide probes have been immobilized at a high density are for example described in U.S. Pat. No. 5,412,087 and in PCT application No. WO 95/11995.

The nucleotide primers according to the invention may be used to amplify any one of the nucleic acids according to the invention, and more particularly all or part of a nucleic acid having the sequences SEQ ID NO 1-5, or alternatively a variant thereof.

Another subject of the invention relates to a method of amplifying a nucleic acid according to the invention, and more particularly a nucleic acid having the sequences SEQ ID NO 1-5 or a fragment or a variant thereof contained in a sample, said method comprising the steps consisting in:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

To carry out the amplification method as defined above, use will be advantageously made of any one of the nucleotide primers described above.

The subject of the invention is, in addition, a box or kit for amplifying a nucleic acid according to the invention, and more particularly all or part of a nucleic acid having the sequences SEQ ID NO 1-5, said box or kit comprising:

a) a pair of nucleotide primers in accordance with the invention, whose hybridization position is located respectively on the 5′ side and on the 3′ side of the target nucleic acid whose amplification is sought;

b) where appropriate, the reagents necessary for the amplification reaction.

Such an amplification box or kit will advantageously comprise at least one pair of nucleotide primers as described above.

The invention is in addition illustrated, without however being limited, by the figures and examples below.

FIG. 1 is a schematic representation of the sites for transcription factors found in humans and mice in the promoter region of the ABCA7 genes.

FIG. 2 illustrates the sequence SEQ ID No. 1. The position of each of the characteristic units for binding to various transcription factors is represented in bold characters, the designation of the transcription factor specific for the corresponding sequence being indicated above the nucleotide sequence.

FIG. 3 illustrates the sequence SEQ ID No. 4. The position of each of the characteristic units for binding to various transcription factors is represented in bold characters, the designation of the transcription factor specific for the corresponding sequence being indicated above the nucleotide sequence.

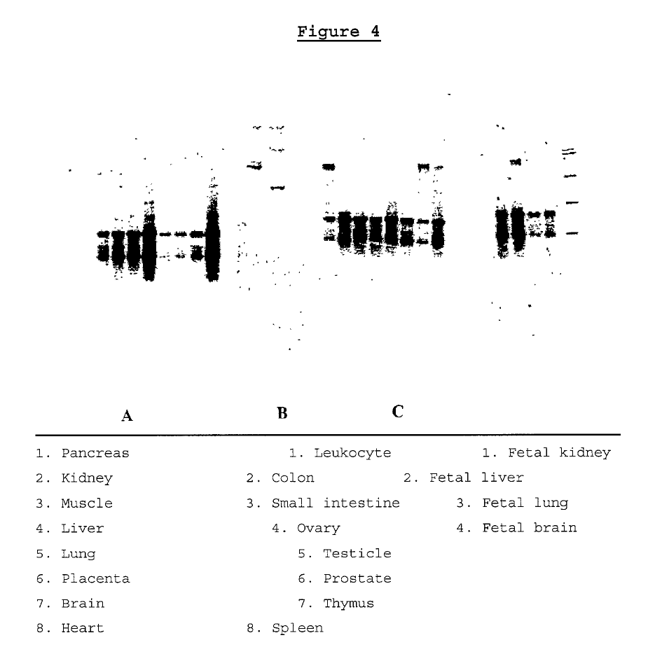

FIG. 4 illustrates the pattern of expression of the human ABCA7 gene on Northern blots of various adult and fetal tissues (Clontech) hybridized with an amplimer produced with the primers SEQ ID No. 9 and 10 (Table 4).

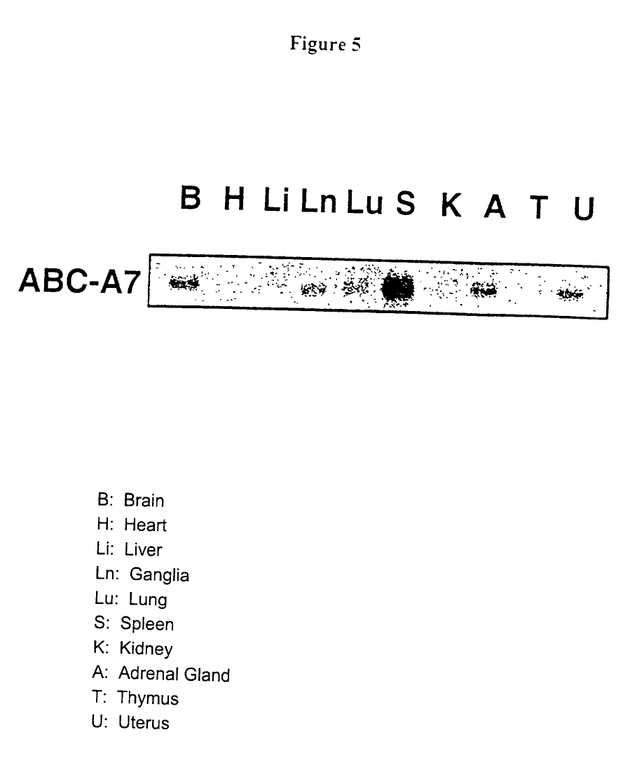

FIG. 5 illustrates the pattern of expression of the murine ABCA7 gene on a Northern blot of various adult tissues hybridized with an amplimer produced with primers specific for the murine transcript.

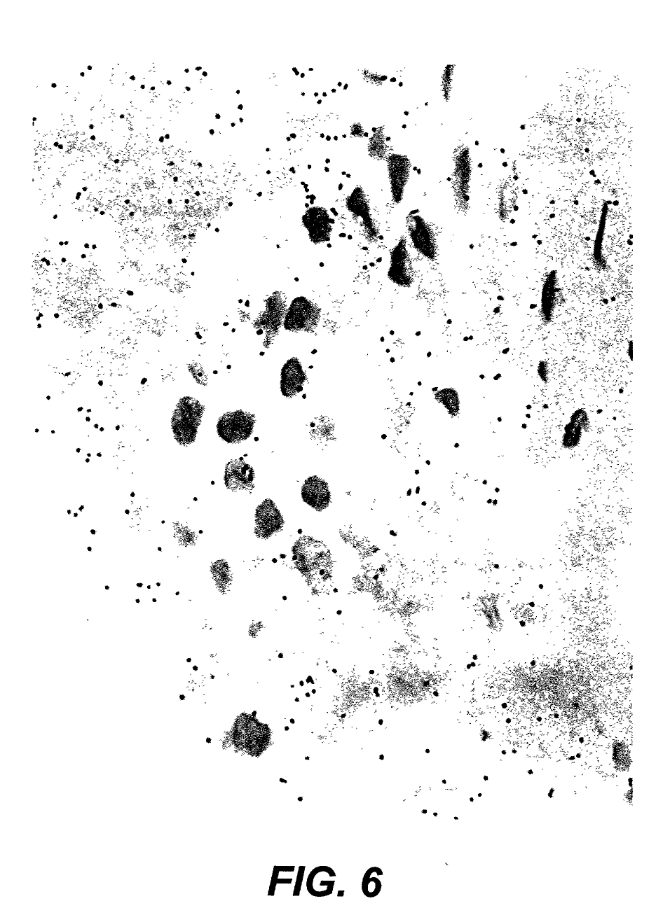

FIG. 6 shows a section of artery showing atherosclerosis and acute inflammation obtained at a below-the-knee amputation from a 92-year-old male. Macrophages in the organizing thrombus and within the inflammatory infiltrate in the adventitia were faintly positive for hybridization.

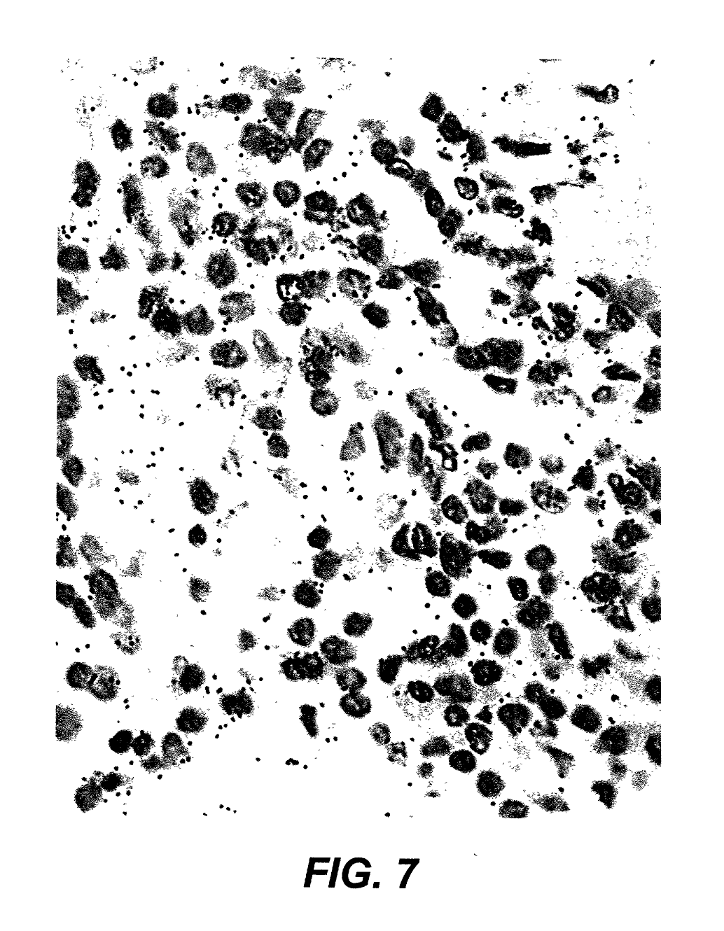

FIG. 7 shows a section of bronchus obtained at autopsy from a 63-year-old asthmatic female. Respiratory epithelium showed faint hybridization. In the submucosal inflammatory infiltrate, small subsets of lymphocytes showed faint hybridization. Occasional, faint hybridization was visible in macrophages.

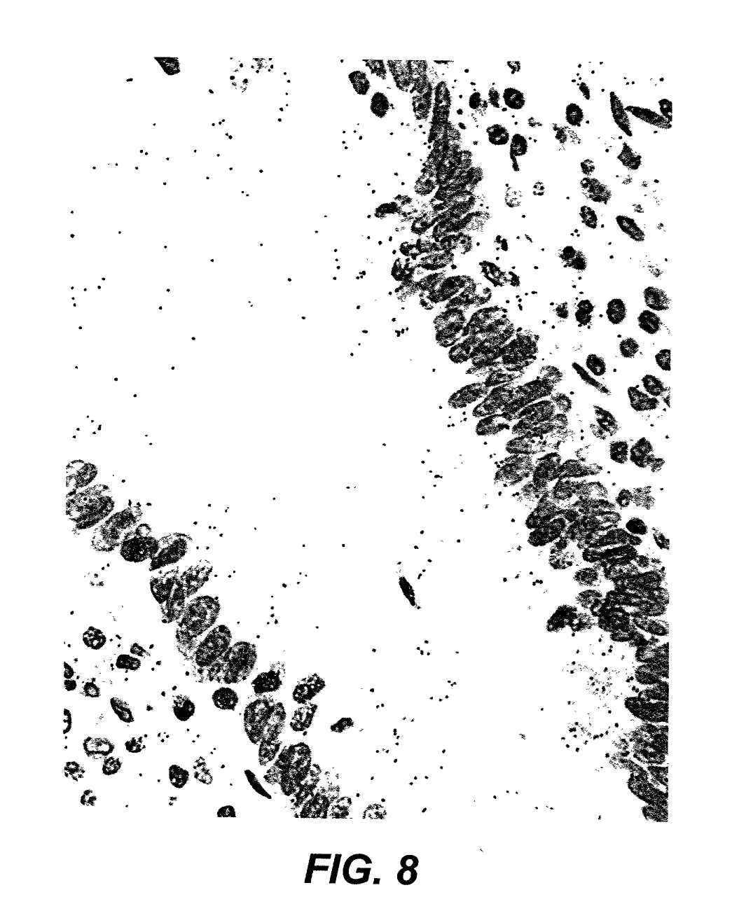

FIG. 8 shows a section of colon obtained at surgery from an 81-year-old female with a clinical diagnosis of Crohn's disease. In the lamina propria, hybridization was identified in macrophages, subsets of lymphocytes, and occasional plasma cells.

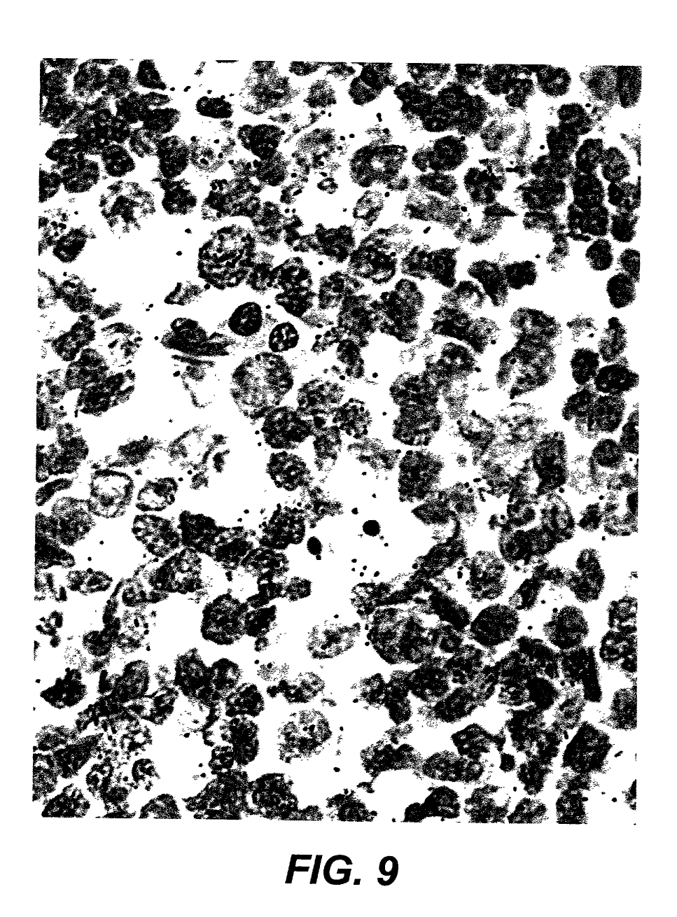

FIG. 9 shows a section of normal lymph node obtained at surgery from a 48-year-old male. Subsets of lymphocytes showed faint hybridization. In reactive germinal centers, subsets of cells showed faint to occasionally moderate hybridization. Scattered throughout the lymph node, cells resembling macrophages were positive.

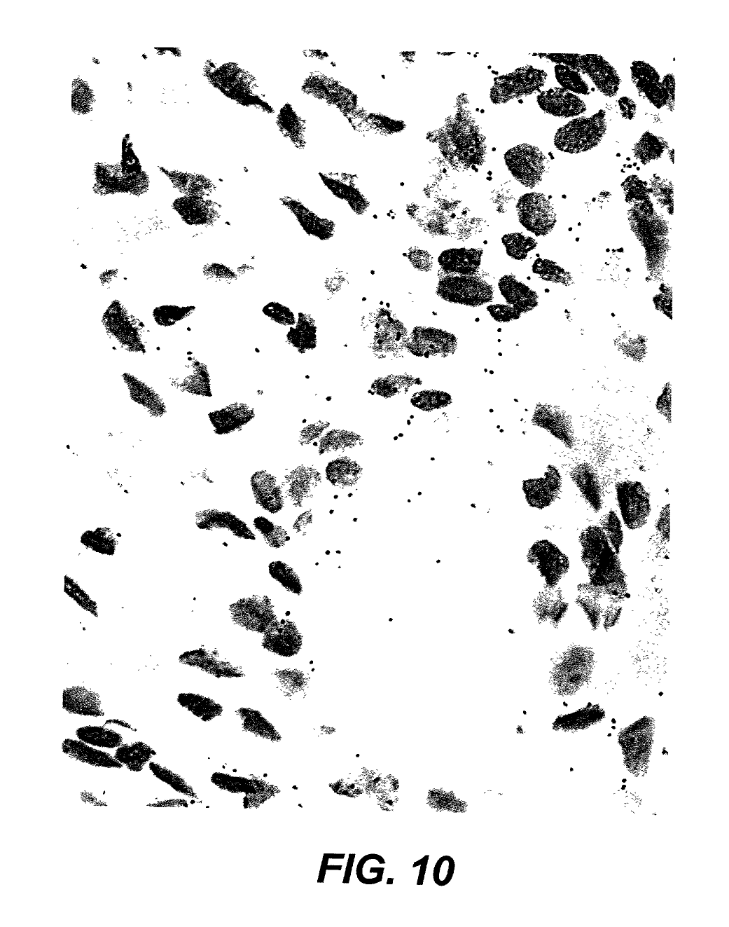

FIG. 10 shows a section of synovium obtained from a 25-year-old female with a clinical diagnosis of rheumatoid arthritis. In most areas, subsynovial histiocytes and macrophages appeared to show stronger hybridization than superficial synoviocytes. In reactive lymphoid follicles, faint to moderate hybridization was also identified in subsets of lymphocytes within germinal centers and within the corona.

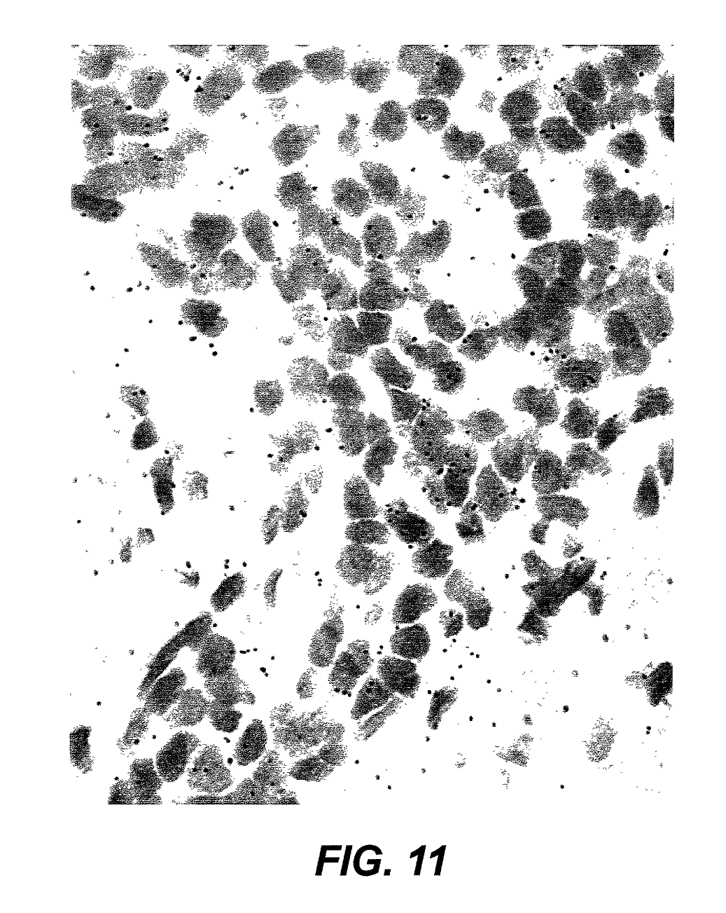

FIG. 11 shows a section of skin obtained at biopsy from a 55-year-old female with a diagnosis of psoriasis. Epidermal keratinocytes showed faint, positive hybridization. In the perivascular inflammatory infiltrate, macrophages were moderately positive. Scattered perivascular lymphocytes also appeared to be positive.

### EXAMPLES

### Example 1

### Determination of the 5′ end of the cDNA for ABCA7

Amplification of the end of the mRNA by RT-PCR (RACE) was carried out using the SMART RACE cDNA amplification kit (Clontech, Palo Alto, Calif.). (PolyA) mRNAs extracted from human spleen tissues were used as template in order to produce a SMART 5′ cDNA library according to the manufacturer's instructions. The first amplification primers and the internal primers were chosen from the cDNA sequence. The amplifications carried out with the internal primers for PCR amplification were cloned. Specific clones were then amplified using primers whose sequences are respectively (CAGGAAACAGCTATGAC) and (GCCAGTGTGATGGATAT) and sequenced on the two strands. Finally, the primers ABCA7 L1 GCGGAAAGCAGGTGTTGTTCAC (SEQ ID No. 11) and ABCA7L2 CGATGGCAGTGGCTTGTTTGG (SEQ ID No. 12) were used to identify the end of the human ABCA7 cDNA.

### Example 2

### Analysis of the Promoter of the Human and Murine ABCA7 Genes

The site of initiation of transcription was located on the promoters of the human and murine genes for ABCA7 using the following three software packages: TSSG and TSSW (Solovyev et al., Ismb (1997) 5, 294-302) and NNPP (Reese M G, et al., 1999). A prediction of the binding sites for the human and murine transcription factors was made using the MatInspector program for searching for motifs (Quandt et al., Nucl. Acid Research (1995) 23(23) 4878-84). The calculation of the scores for each binding site for the transcription factors was made using the following formula: (Of−Tf)/(Tf)1/2, in which “Of” is the frequency of observation of a motif and “Tf” is the calculated frequency of a consensus motif. In order to separate the motifs which are not considered to be relevant, a first filtration step was performed by adjusting the Matlnspector program “template similarity” score above 0.85 and the “core similarity” score above 0.99. Finally, a comparative analysis of the inter-species promoters was made as described by Werner T (Models for prediction and recognition of eukaryotic promoters, Mammalian Genome (1999) 10: 168-175) in order to define the transcription modules comprising sites having a similar motif and present both on the human and murine sequences of the sequence upstream of the ABCA7 gene.

### Example 3

### Preferential Expression of Human and Murine ABCA7 Genes in Hematopoietic Tissues

The profile of expression of the polynucleotides according to the present invention was determined according to the protocols for PCR-coupled reverse transcription and Northern blot analysis described in particular by Sambrook et al. (ref. C S H Sambrook, J., Fritsch, E. F., and Maniatis, T. (1989). “Molecular Cloning: A Laboratory Manual,” 2nd ed., Cold Spring Harbor Laboratory Press, Cold Spring Harbor, N.Y.).

For example, in the case of an analysis by reverse transcription, a pair of primers was synthesized from each of the complete cDNAs of the human and murine ABCA7 genes in order to detect the corresponding cDNAs. The sequences of these primers are presented in Table 4.

The polymerase chain reaction (PCR) was carried out on cDNA templates corresponding to reverse transcribed polyA⁺ mRNAs. The reverse transcription to cDNA was carried out with the enzyme SUPERSCRIPT II (GibcoBRL, Life Technologies) according to the conditions described by the manufacturer.

The polymerase chain reaction was carried out according to standard conditions, in 20 μl of reaction mixture with 25 ng of cDNA preparation. The reaction mixture was composed of 400 μM of each of the dNTPs, 2 units of Thermus aquaticus (Taq) DNA polymerase (Ampli Taq Gold; Perkin Elmer), 0.5 μM of each primer, 2.5 mM MgCl2, and PCR buffer. Thirty PCR cycles (denaturing 30 s at 94° C., annealing of 30 s divided up as follows during the 30 cycles: 64° C. 2 cycles, 61° C. 2 cycles, 58° C. 2 cycles and 55° C. 28 cycles and an extension of one minute per kilobase at 72° C.) were carried out after a first step of denaturing at 94° C. for 10 min in a Perkin Elmer 9700 thermocycler. The PCR reactions were visualized on agarose gel by electrophoresis. The cDNA fragments obtained may be used as probes for a Northern blot analysis and may also be used for the exact determination of the polynucleotide sequence.

In the case of a Northern Blot analysis, a cDNA probe produced as described above was labeled with ³²P by means of the DNA labeling system High Prime (Boehringer) according to the instructions indicated by the manufacturer. After labeling, the probe was purified on a Sephadex G50 microcolumn (Pharmacia) according to the instructions indicated by the manufacturer. The labeled and purified probe was then used for the detection of the expression of the mRNAs in various tissues.

The Northern blot containing samples of RNA of various human tissues (Multiple Tissue Northern or MTN; references (Human II, 7759-1, Human 7760-1, and Human Fetal II 7756-1, Clontech) was hybridized with the designated specific labeled probe for ABCA7 (2637-4881 bp).

The protocol followed for the hybridizations and washes may be either directly that described by the manufacturer (Instruction manual PT1200-1) or an adaptation of this protocol using methods known to persons skilled in the art and described for example in F. Ausubel et al. (Currents Protocols in Molecular Biology, Green Publishing Associates and Wiley Interscience NY (1989). It is thus possible to vary, for example, the prehybridization and hybridization temperatures in the presence of formamide.

For example, it may be possible to use the following protocol:

1—Membrane Competition and PREHYBRIDIZATION:

Mix: 40 μl salmon sperm DNA (10 mg/ml)

+40 μl human placental DNA (10 mg/ml)\

Denature for 5 min at 96° C., then immerse the mixture in ice.

Remove the 2× SSC and pour 4 ml of formamide mix in the hybridization tube containing the membranes.

Add the mixture of the two denatured DNAs.

Incubate at 42° C. for 5 to 6 hours, with rotation.

2—Labeled Probe Competition:

Add to the labeled and purified probe 10 to 50 μl Cot I DNA, depending on the quantity of repeat sequences.

Denature for 7 to 10 min at 95° C.

Incubate at 65° C. for 2 to 5 hours.

3—Hybridization:

Remove the prehybridization mix.

Mix 40 μl salmon sperm DNA+40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4—Washes:

One wash at room temperature in 2× SSC, to rinse.

Twice 5 minutes at room temperature 2× SSC and 0.1% SDS.

Twice 15 minutes 0.1× SSC and 0.1% SDS at 65° C.

After hybridization and washing, the blot was analyzed after overnight exposure in contact with a phosphorus screen revealed with the aid of Storm (Molecular Dynamics, Sunnyvale, Calif.).

The results presented in FIG. 5 show that the mouse ABCA7 gene is expressed in the adult tissues. A larger quantity of murine ABCA7 mRNA was detected in the hematopoietic tissues such as the spleen and thymus, which is consistent with the expression of ABCA7 that was observed in the myelomonocytic and lymphocytic lines. No expression of the ABCA7 gene was detected in the fibroblastic cell lines.

FIG. 4 shows a similar pattern of expression of the human ABCA7 gene with however a strong hybridization signal in the fetal liver.

### Example 4

### Analysis of the Gene Expression Profile for Dysfunctions in the Metabolism of Lipids, or in Inflammation Signaling

The verification of the impairment of the level of expression of the ABCA7 gene may be determined by hybridization of these sequences with probes corresponding to the mRNAs obtained from hematopoietic tissues from subjects who are affected or otherwise, according to the methods described below:

1. Preparation of the Total RNAs, of the poly(A)⁺ mRNAs and of cDNA Probes

The total RNAs are obtained from hematopoietic tissues from normal or highly affected subjects by the guanidine isothiocyanate method (Chomczynski et al., Anal Biochem (1987) 162:156-159). The poly(A)⁺ mRNAs are obtained by affinity chromatography on oligo(dT)-cellulose columns (Sambrook et al., (1989) Molecular cloning: a laboratory manual. 2ed., Cold Spring Harbor Laboratory, Cold Spring Harbor, N.Y.) and the cDNAs used as probes are obtained by RT-PCR (DeRisi et al., Science (1997) 278:680-686) with oligonucleotides labeled with a fluorescent product (Amersham Pharmacia Biotech; CyDye T M).

2. Hybridation and Detection of the Expression Levels

The glass slides containing the sequences according to the present invention corresponding to the ABCA7 gene are hydridized with the nucleotide probes prepared from the messenger RNA of the cell to be analyzed. The use of the Amersham/molecular Dynamics system (Avalanche Microscanner TM) allows the differential quantification of the expressions of the products of sequences on healthy or affected cell type.

### Example 5

### Test Intended for the Screening of Molecules Activating or Inhibiting the Expression of the ABCA7 Gene

The screening test makes it possible to identify a sequence capable of modulating the activity of synthesis of the ABCA7 protein.

5.1 Construction of the Expression Plasmids Containing a Nucleic Acid for Regulating the Human ABCA7 Gene

The region of the acid for regulating the human ABCA7 gene ranging from the nucleotide at position −1111 up to the nucleotide at position −1, relative to the site of initiation of transcription, may be amplified by the PCR technique with the aid of the pair of primers specific for the region described above from human genomic DNA present in a BAC vector of a human BAC vector collection.

The amplified DNA fragment is digested with restriction endonuclease Sal 1, then inserted into the vector PXP1 described by Nordeen et al. (Bio Techniques, (1988) 6:454-457), at the level of the Sal 1 restriction site of this vector. The insert is then sequenced.

5.2 Cell Culture and Transfection

Cells of the CHO or HELA line (ATCC, Rockville, Md., USA) are cultured in the E-MEM (Minimum Essential Medium with Earle's Salts) medium supplemented with 10% (v/v) fetal calf serum (BioWhittaker, Walkersville, Md.). Approximately 1.5×105 cells are distributed into each of the wells of a 12-well culture plate (2.5 cm), and are cultured up to about 50-70% confluence, and then cotransformed with 1 μg of plasmid Sal-Lucif and 0.5 μg of the control vector pBetagal (CloneTech Laboratories Inc., Palo Alto, Calif., USA) using the Superfectin Reagent Kit (QIAGEN Inc., Valencia, Calif., USA). Two hours after the addition of the DNA, the culture medium is removed and replaced with complete AMEM (Minimum Essential Medium Eagle 's Alpha Modification) medium. After a period of twenty hours, the cells are placed in fresh medium of the DMEM (Dulbecco's Minimum Essential Medium) type supplemented with 2 μg/ml of glutamine, 100 units/ml of streptomycin and 0.1% of bovine serum albumin (BSA, Fraction V), in the presence or otherwise of molecules at various concentrations.

The cells are recovered 16 hours after the last change of medium using a Lysis Solution obtained from the Tropix Luciferase Assay Kit (Tropix Inc., Bedford, Mass., USA). The cellular lysate is divided into aliquot fractions which are used to quantify the proteins using the MicroBCA Kit (Pierce, Rockford, Ill., USA) as well as to quantify the production of luciferase and beta-galactosidase using the Tropix Luciferase Assay Kit and Galacto-Light Plus Kit, respectively. The tests are carried out according to the manufacturer's recommendations. The molecules active on the ABCA7 promoter are then selected according to the ratio “luciferase activity/beta-galactosidase activity”

### Example 6

### In situ Hybridization Study Using ABCA7 Probe

Serial tissue sections from archival paraffin samples were hybridized with radiolabeled cRNA probes corresponding to an ABCA7 fragment. The ABCA7 fragment, which corresponds to nucleotides 594 through 1055 of GenBank sequence NM_{—}019112, was subcloned into pCRII (Invitrogen) and transcribed in vitro with SP6 (antisense) and T7 (sense) RNA polymerases in the presence of ³⁵S-uridine 5′-triphosphate. After transcription, the probes were column-purified and separated by electrophoresis on a 5% polyacrylamide gel to confirm size and purity.

Tissue sections were digested with proteinase K and hybridized with the probes at a concentration of approximately 3.5×10⁷ dpm/ml for 18 hours at 65° C. Following hybridization, the slides were treated with RNAse A and washed stringently in 0.1× SSC at 70° C. for 2 hours. The slides were then coated with Kodak NTB-2 emulsion, exposed 7 days at 4° C., and developed using Kodak D-19 Developer and Fixer. Slides were stained with hematoxylin and eosin (H&E) and imaged using a DVC digital photo camera coupled to a Nikon microscope.

Hybridization signals appeared increased in markedly inflamed tissue, and were identified consistently in subsets of macrophages and lymphocytes across all samples. In macrophages, only subsets of cells showed hybridization. For example, in asthmatic samples, the submucosal inflammatory infiltrate contained positive macrophages (FIG. 7). Macrophages were also positive within inflammatory infiltrates and granulomas of Crohn's disease (FIG. 8), in psoriasis samples (FIG. 11), and in subsynovial histiocytes of rheumatoid arthritis (FIG. 10). Similarly, subsets of lymphocytes were also positive within lymphoid aggregates, germinal centers (FIG. 9), inflammatory infiltrates of Crohn's disease (FIG. 8), in psoriasis (FIG. 11), and in rheumatoid arthritis samples (FIG. 10).

TABLE 1
Sites, scores, consensus and positions relative to the site of initiation of
transcription (TSS) predicted by the NNPP, TSSG and TSSW software packages in humans

| | | | | | | Core | |
|---|---|---|---|---|---|---|---|
| | | | | | Position/ | simi- | Template |
| Filtration | Site | Consensus | Sequence | Z score | TSS(bp) | larity | similarity |
| Comparative | GFI1_01 | NNNNNAAATCANNGNNNN | gccactatAATCgaqayackaga | 3 669779 | −569 | 1 00 | 0 88 |
| analysis | | NNNN | | | | | |
| between | HNF3B_01 | NNNTRTTTRYTY | gaaTGTTggccc | 3 978804 | −547 | 0 99 | 0 85 |
| species | CEBPB_01 | RNRTKNNGMAAKNN | cgttcglGGAAlga | 1 857489 | −498 | 0 87 | 0 85 |
| | CEBPB_01 | RNRTKNNGMAAKNN | atctaglGGAAccc | 1 857489 | −469 | 0 87 | 0 85 |
| | NFI_Q6 | NNTTGGCNNNNNCCNNN | gccTGGCagcccrgggg | 1 651312 | −402 | 1 00 | 0 86 |
| | AP4_Q6 | CWCAGCTGGN | lgCAGCcggl | 12 133646 | −340 | 1 00 | 0 85 |
| | NFKAPPAB_01 | GGGAMTTYCC | GGGAcctgcc | 9 285691 | −260 | 1 00 | 0 90 |
| | NFY_Q6 | TRRCCAATSRN | cgcCCAlagc | 6 200634 | −106 | 1 00 | 0 89 |
| Z score >= | AHRARNT_01 | KNNKNNTYGCGTGCMS | cgalyagggCGTGctt | 10 450420 | −1065 | 1 00 | 0 87 |
| 1.96 | CDPCR3HD_01 | NATYGATSSS | ggGATCaagg | 2 474120 | −1004 | 1 00 | 0 87 |
| | IK1_01 | NNNTGGGAATRCC | caagGGGAaaatg | 14 484154 | −999 | 1 00 | 0 87 |
| | NFY_Q6 | TRRCCAATSRN | accaIIGGgag | 6 200634 | −978 | 1 00 | 0 87 |
| | LYF1_01 | TTTGGGAGR | attGGGAgg | 7 208594 | −975 | 1 00 | 0 91 |
| | BARBIE_01 | ATNNAAAGCNGRNGG | agcaAAAGctgaagc | 32 363969 | −963 | 1 00 | 0 91 |
| | E47_02 | NNNMRCAGGTGTTMNN | agccaCAGGlgaglcl | 15 631450 | −951 | 1 00 | 0.68 |
| | MYOD_01 | SRACAGCTGKYG | ccaCAGGlyagl | 32 908282 | −949 | 1 00 | 0 89 |
| | LMO2COM_01 | SNNCAGGTGNNN | ccaCAGGlagt | 2 232208 | −949 | 1 00 | 0 95 |
| | TH1EA7_01 | NNNNGNRTCTGGMWTT | aggtgagtCTGGgt | 16 677521 | −945 | 1 00 | 0.91 |
| | GFI1_01 | NNNNNNAAATCANNGNNNN | gglggatqaatGATTtqaggg | 3 609729 | −934 | 1 00 | 0 93 |
| | | NNNN | | | | | |
| | NRF2_01 | ACCGGAACNS | ggcTTCCtgg | 6 109000 | −883 | 1 00 | 0 85 |
| | NFKB_Q6 | NGGGGAMTTTCCNN | gaggcagtTCCClc | 26 126380 | −861 | 1 00 | 0 88 |
| | CREL_01 | SGGRNWTTCC | aggcagTTCC | 2 943267 | −860 | 1 00 | 0 90 |
| | NFKAPPAB_01 | GGGAMTTYCC | ggcaglTCCC | 9 285691 | −850 | 1 00 | 0 91 |
| | IK1_01 | NNNTGGGAATRCC | gcaglTCCClcaa | 14 484154 | −858 | 1 00 | 0 89 |
| | STAT_01 | TTCCCRKAA | TTCCctcaa | 6 281497 | 854 | 1 00 | 0 91 |
| | BARBIE_01 | ATNNAAAGCNGRNGG | ccatgagCTTTggct | 32 363969 | −838 | 1 00 | 0 90 |
| | USF_Q6 | GYCAGGTGNG | gtctCGTGgc | 5 390268 | −802 | 1 00 | 0 94 |
| | AP2_Q6 | MKCCCSCNGGCG | IcCCCGttggcg | 7 064136 | −777 | 1 00 | 0 86 |
| | VMYB_01 | AAYAACGGNN | cccCGTTgggc | 4 360540 | −776 | 1.00 | 0 89 |
| | TATA_01 | STATAAAWRNNNNNN | aaccctaTTTAtcc | 7 166360 | −765 | 1 00 | 0.87 |
| | GATA_C | NGATAAGNMNN | rctatTTATCc | 2 004465 | −761 | 1 00 | 0 93 |
| | GATA1_03 | NNNNNGAAANNGN | ctaltTATCctcaa | 2 776354 | 700 | 1 00 | 0 94 |
| | VMYB_01 | AAYAACGGNN | cccAACGgca | 4 360548 | 743 | 1 00 | 0 91 |
| | AP2_Q6 | MKCCSCNGGCG | ctgccgCGGGag | 7 064136 | −723 | 1 00 | 0 87 |
| | AHRARNT_01 | KNNKNNTYGCCTGCMS | cccCACGcctckact | 10 450429 | −707 | 1 00 | 0 85 |
| | BARBIE_01 | ATNNAAAGCNGRNGG | cttcAAAGctgtgga | 32 363969 | −680 | 1 00 | 0 88 |
| | AP4_Q6 | CWCAGCTGGN | caaaGCTGg | 13 133646 | −677 | 1 00 | 0 87 |
| | AHRARNT_01 | KNNKNNTYGCGTGCMS | ccaCACGctccattt | 10 450429 | −664 | 1 00 | 0 87 |
| | HFH1_01 | MAWTGTTATWI | aagaGTTattt | 51 812065 | −629 | 1 00 | 0 88 |
| | IK1_01 | NNTGGAATRCC | gagtGGGAaacgg | 14 484154 | −603 | 1 00 | 0 89 |
| | VMY8_01 | AAYAACGGNN | ggaAACGggt | 4 360548 | −598 | 1 00 | 0 89 |
| | CREL_01 | SGGRNYWTCC | cgggttTTCC | 6 194143 | −593 | 1 00 | 0 98 |
| | JFKAPPAB65_01 | GGGRATTTCC | cgggttTTCC | 28 315415 | −593 | 1 00 | 0 95 |
| | GFI1_01 | NNNNNNNNAAATCANNGNNN | tlcctcaaAATCagggtagcatt | 3 669729 | −587 | 1 00 | 0 95 |
| | | NNNN | | | | | |
| | STAT_01 | TTCCCRKAA | TTCCtcaaa | 6781497 | −587 | 1 00 | 0 88 |
| | STAT_01 | TTCCCRKAA | ttcgGCAA | 6 281497 | −496 | 1 00 | 0 92 |
| | BARBIE_01 | ATNNAAAGCNGRNNGG | accctaCTTacag | 32 363909 | −459 | 1 00 | 0 85 |
| | TH1E47_01 | NNNNGNRTCTGGMWTT | agtcCCAGagtctgga | 16 677521 | −432 | 1 00 | 0 88 |
| | TH1E47_01 | NNNNGNRTCTGGMWTT | cccagagtCTGGacta | 16 677521 | −429 | 1 00 | 0 90 |
| | AP4_Q6 | CWCAGCTGGN | gaCAGCgggg | 12 133646 | −385 | 1 00 | 0 90 |
| | IK1_01 | NNNTGGGAATRCC | cagaGGGAactcc | 14 484154 | −374 | 1 00 | 0.90 |
| | CHOP_01 | NNRIGCAATMCC | tccTGCAattcgg | 22 328386 | −364 | 1 00 | 0.87 |
| | AP4_Q6 | CWCAGCTGGN | cggcGCTGcg | 24 429942 | −354 | 1 00 | 0.88 |
| | AP4_Q5 | NNCAGCTGNN | cggaGCTGcg | 3 791000 | −354 | 1 00 | 0 92 |
| | CHOP_01 | NNRTGCAATMCCC | cggtatTGCAgcc | 22 328386 | −346 | 1 00 | 0 93 |
| | HLF_01 | RTTACRYAAT | GTTAacaac | 12 961423 | −332 | 1 00 | 0 85 |
| | IK1__01 | NNNTGGGAATRCC | ctcgTTCCCggag | 14 484154 | 285 | 1 00 | 0 88 |
| | SP1_Q6 | NGGGGCGGGGYN | ggagGGCGgcctg | 11 119144 | −276 | 1 00 | 0 86 |
| | NFKB_Q6 | NGGGGAMTTTCCNN | ctGGGAcctgccgg | 26 126380 | −262 | 1 00 | 0.87 |
| | AP4_Q6 | CWCAGCTGGN | cgCAGCtacg | 24.429942 | −146 | 1 00 | 0 88 |
| | AP4_Q5 | NNCAGCTGNN | cgCAGCtccg | 3 791000 | −146 | 1 00 | 0 92 |
| | ATF_01 | CNSTGACGTNNNYC | gagTGACgggcagg | 8 675151 | −121 | 1 00 | 0 86 |
| | AP1FJ_Q2 | RSTGACTNNNW | agTGACgggca | 5 905504 | −120 | 1 00 | 0 91 |
| | AP1_Q2 | RSTGACTNMNW | agTGACgggca | 5 905504 | 120 | 1 00 | 0 89 |
| | CAAT_01 | NNNRRCCAATSA | gtcgcCCAAtag | 4 415584 | −108 | 1 00 | 0 86 |
| | AHRARNT_01 | KNNNKNTYGCGTGCMS | caatagcagCGTGcag | 10.450429 | −102 | 1 00 | 0 90 |
| | AHRARNT_01 | KNNKNNTYCGCTGCMS | aggcaggggCGTGccc | 10 450429 | −86 | 1 00 | 0 92 |
| | GC_01 | NRGGGCCGGGGCNK | aaggCGCGgcggagc | 15.933816 | −28 | 1 00 | 0 92 |
| | SP1_Q6 | NGGGGCGGGGYN | aaggCGCGgcgcg | 11 119144 | −28 | 1 00 | 0 93 |
| | AP4_Q6 | CWCAGCTGGN | gcctGCTGct | 12 133646 | −9 | 1 00 | 0 86 |
| | SP1_Q6 | NGGGGGCGGGGYN | gctgGGCGgagag | 11 119141 | −2 | 1 00 | 0 90 |
| | GC_01 | NRGGGGCGGGGCNK | gctgGGCGqagga | 15 933816 | −2 | 1 00 | 0 87 |
| | IK1_01 | NNNTGGGAATRCC | cggaGGGAaggcg | 14 481151 | 4 | 1 00 | 0 87 |
| | AP4_Q6 | GWCAGCTGGN | aagaCCTGLag | 12 133646 | 19 | 1 00 | 0 89 |
| | IK1_01 | NNNTGGGAATRCC | gagaGGGAagacag | 14 484151 | 56 | 1 00 | 0 87 |
| | IK1_01 | NNNTGGGAATRCC | caagTCCCtggg | 14 481151 | 110 | 1 00 | 0 87 |
| | IK1_01 | NNNTGGGAATRCC | ccctCGGAattag | 14 484154 | 116 | 1 00 | 0 92 |
| | TST1_01 | NNKGAWTWANANTNN | tgggAATThagggggl | 6 882911 | 119 | 1 00 | 0 87 |
| | NKX25_02 | CWTAATTG | gaATTAqg | 5 675005 | 122 | 1 00 | 0 91 |
| | AP1_Q2 | RSTGACTNMNW | tcTGACctccl | 5 905504 | 140 | 1 00 | 0 86 |
| | AP1FJ_02 | RSTGACTNNNW | tcTGACctccl | 5 905504 | 140 | 1 00 | 0 90 |
| | RORA1_01 | NWAWNNAGGTCAN | ctGACCtccttcc | 15 381241 | 141 | 1 00 | 0 94 |
| | RORA2_01 | NWAWNTAGGTCAN | ctGACCtccttcc | 33 905118 | 141 | 1 00 | 0 85 |
| | NRF2_01 | ACCGGMGNS | tccTTCCggI | 6 109080 | 147 | 1 00 | 0 96 |
| | ATF_01 | CNSTGACGTNNNYG | IgITGACgacggcl | 8 675151 | 160 | 1 00 | 0 91 |
| | CREB_04 | NSTGACGTTTMANN | gITGACgacggc | 5 543914 | 161 | 1 00 | 0 87 |
| | AP1_Q2 | RSTGACTNMNW | glTGACgacgg | 5 906504 | 161 | 1 00 | 0 89 |
| | AP1FJ_02 | RSTGACINMNW | gtTGACgacgg | 5 905504 | 161 | 1 00 | 0 90 |
| | GFI1_01 | NNNNNNAAATCANNGNNNN | gaattgatcactGATTclcaagg | 3 660729 | 174 | 1 00 | 0 92 |
| | | NNNN | | | | | |
| | CDPCR3HD_01 | NATYGATSSS | aattGATCac | 2 474120 | 175 | 1 00 | 0 97 |
| | TH1E47_01 | NNNNGNRTCTGGMW11 | tcggacaCTGGgacc | 16 677521 | 203 | 1 00 | 0 89 |
| | TAL1ALPHAE47_{—} | NNNAACAGATCGKTNNN | tcggacaTCTGggacc | 43 162108 | 203 | 1 00 | 0 86 |
| | 01 | | | | | | |
| | TAL1BETAE47_{—} | NNNAACAGATGKTNWN | tcgggacaTCTCggacc | 43 162108 | 203 | 1 00 | 0 87 |
| | 01 | | | | | | |
| | E47_02 | NNNMRCAGGTGTTMNN | tcacaacaCCTGagcc | 15 631450 | 239 | 1 00 | 0 90 |
| | E47_01 | NSNGCAGGTGKNGNN | tcacaacCTGCagc | 6 708124 | 239 | 100 | 0 97 |
| | LMO2COM_01 | SNNCAGGTGNNN | acacaCCTGcag | 2 232288 | 241 | 1 00 | 0 97 |
| | MYOD_01 | SRACAGGTGKYG | cacaCCTGcag | 32 908282 | 241 | 1 00 | 0 87 |
| | VMYB_01 | AAYAACGGNN | gccCGTTaga | 4 3611548 | 259 | 1 00 | 0 92 |
| | SRY_02 | NWWAACAAWANN | IcIlACAAaIgg | 8 473458 | 293 | 1 00 | 0 86 |
| | TH1E47_01 | NNNNGNRTCTGGMWTT | tcccCCAGatcctaag | 16 677521 | 320 | 1 00 | 0 88 |
| | E4BP4_01 | NRTTAYGTAAYN | cttgalGTAAag | 12 678534 | 341 | 1 00 | 0 86 |
| | VBP_01 | GTTACRTNAN | ttgatGTAAa | 5 053244 | 342 | 1.00 | 0 92 |
| | CREL_01 | SGGRNWTTCC | GGAAagaacc | 2 943267 | 352 | 1 00 | 0 85 |
| | VBP_01 | GTTACRTNAN | ctggcGTAAg | 5 053244 | 362 | 1 00 | 0 86 |
| | TH1E47_01 | NNNNGNRTCTGGMWIT | gtacagggtCTGGgtct | 16 677521 | 367 | 1 00 | 0 92 |
| | AP1FJ_Q2 | RSTGACTNMNW | qctgGICAcc | 9 018855 | 308 | 1 00 | 0 92 |
| | AP1_Q2 | RSTGACTNMNW | gctgGTCAcc | 9 018855 | 398 | 1 00 | 0 89 |
| | AP1_Q4 | RSTCACTMANN | gcctgGTCAcc | 13 148580 | 398 | 1 00 | 0.86 |
| | ER_Q6 | NNARGNNANNNTGACCWNN | cctgGTCAcctttagaca | 11 677290 | 490 | 1 00 | 0 88 |
| | ELK1_01 | NNNACMGGAAGTNGNN | agcaaTTCCggccc | 15 1614525 | 412 | 1 00 | 0 86 |
| | AP1FJ_Q2 | RSTGACTNMNW | cctcGTCAgr | 9 018855 | 420 | 1 00 | 0 93 |
| | API_Q2 | RSTGACTNMNW | cctctGTCAgr | 9 018855 | 426 | 1 00 | 0 92 |
| | AP1_Q4 | RSTGACTMANN | cctctGTCAgr | 13 148586 | 426 | 1 00 | 0 89 |
| | GFI1_01 | NNNNNNAAATCANNGNNNN | ctgtcagcgtaGATTctccatct | 3 660729 | 429 | 1 00 | 0 86 |
| | | NNNN | | | | | |
| | ATF_01 | CNSTGACGINNNYC | tgtcagcGTCAgal | 8 867151 | 430 | 1 00 | 0 90 |
| | CREB_Q4 | NSTGACGTMANN | gtcagcGTCAga | 11 262690 | 431 | 1 00 | 0 88 |
| | CREB_Q2 | NSTGACGTAANN | gtcagcGTCAga | 17 782892 | 411 | 1 00 | 0 86 |
| | AP1FJ_Q2 | RSTGACTNMNW | IrcagcGTCAga | 5 905504 | 432 | 1 00 | 0 90 |
| | AP1_Q2 | RSTGACTNMNW | IcagcGTCga | 5 905604 | 432 | 1 00 | 0 88 |
| | TAL1BETAE47_{—} | NNNAACAGATGKTNNN | ttctccaTCTGtgta | 64 766308 | 413 | 1 00 | 0 88 |
| | 01 | | | | | | |
| | TAL1BETAITF2_{—} | NNNMCAGATGKTNNN | ttctccgTCIGgtra | 64 766306 | 443 | 1 00 | 0 87 |
| | 01 | | | | | | |
| | TAL1ALPHAE47_{—} | NNAACAGATGKTNNN | ttctccatCTGgtca | 64 765306 | 443 | 1 00 | 0 69 |
| | 01 | | | | | | |
| | AP1FJ_Q2 | RSTGACTNMNW | tctgtGTCAga | 9 018855 | 450 | 1 00 | 0 93 |
| | AP1_Q4 | RSTGACTNMANN | tctgCTCAga | 13 148586 | 450 | 1 00 | 0 88 |
| | AP1_Q2 | RSTGACTMANN | tctgtGTCAga | 9 018855 | 450 | 1 00 | 0 91 |
| | CDPCR3HD_01 | NATYGATSSS | aalaGATCag | 2 474120 | 480 | 1 00 | 0 95 |
| | GFI1_01 | NNNNNAAATCANNGNNNN | agctcggAATCgcgactccag | 3 669729 | 483 | 1 00 | 0 89 |
| | | NNNN | g | | | | |
| | AP1_Q2 | RSTGACNMNW | gcTGACIccag | 9 018856 | 495 | 1 00 | 0 94 |
| | AP1FJ_Q2 | RSTGACTNMNW | gcTGACIccag | 9 015855 | 495 | 1 00 | 0 94 |
| | AP1_Q4 | RSTGACTMANN | gcTGACIccag | 13 148586 | 495 | 1 00 | 0 91 |
| | GATA1_03 | NNNNNGATAANNGN | gtctcTATCccagc | 2 776354 | 508 | 1 00 | 0 89 |
| | AP1FJ_Q2 | RSTGACTNMNW | ccTGACtctt | 9 018855 | 520 | 1 00 | 0 92 |
| | AP1_Q2 | RSTGACTNMNW | ccTGACtctt | 9 018855 | 529 | 1 00 | 0 91 |
| | BARBIE_01 | ATNNAAAGCNGRNGG | cctgactCTTctct | 32 363069 | 629 | 1 00 | 0 86 |
| | AP1_Q4 | RSTGACTMANN | ccTGACtctt | 13 148588 | 529 | 1 00 | 0 88 |
| | TH1E47_01 | NNNNGNRTCTGGMWTT | ctctCTGGctcc | 16 677521 | 534 | 1 00 | 0 85 |
| | CP2_01 | GCNMNAMCMAG | CTGGctcccgc | 3 245733 | 542 | 1 00 | 0 90 |
| | AP2_Q6 | MKCCCSCNGGCG | ctCCCGcggtcc | 7 064136 | 546 | 1 10 | 0 88 |
| | GFI1_01 | NNNNNNMATCANNGNNN | gtccctctgagGATTaatgtacd | 3 669729 | 554 | 1 00 | 0 85 |
| | | NNNN | | | | | |
| | AP4_Q6 | CWCAGCTGGN | cagaGCTgg | 12 133646 | 590 | 1 00 | 0 87 |
| | AP4_01 | WGARYCAGCTGYGGNCNK | gtgcctccaGCTGggcaa | 178 524329 | 603 | 1 00 | 0 86 |
| | RFX1_01 | NNGTNRCNNRGYAACNN | ctccagclygGCAActg | 7 226828 | 607 | 1 00 | 0 89 |
| | AP4_Q6 | CWCAGCTGGN | tccaGCTGgg | 18281794 | 608 | 1 00 | 0 97 |
| | AP4_Q5 | NNCAGCTGNN | tccaGCTGgg | 2 628244 | 608 | 1 00 | 0 96 |
| | AP4_Q6 | GWCAGC1GGN | tcCAGCtggg | 16 281794 | 600 | 1 00 | 0 93 |
| | AP4_Q5 | NNCAGCTGNN | tcCAGCtggg | 2 628244 | 608 | 1 00 | 0 94 |
| | E47_01 | NSNGCAGGTGKNCNN | clgggcaaCTGCctg | 6 708124 | 613 | 1 00 | 0 87 |
| | SREBP1_02 | KATCACCCCAC | gtgggGTGAta | 30 499201 | 682 | 1 00 | 1 00 |
| | GATA1_03 | NNNNNGATAANNGN | gggglGATAgIcca | 2 776354 | 684 | 1 00 | 0 91 |
| | OLF1_01 | NNCNATCCCYNGRGARN | agcaclTCCCctgggcylgtga | 64 601270 | 607 | 1 00 | 0 89 |
| | | KGN | | | | | |
| | IK1_01 | NNNTGGGAATRCC | gcacITCCCctgg | 14 484154 | 098 | 1 00 | 0 87 |
| | NRF2_01 | ACCGGAAGNS | cacTTCCcct | 6 109000 | 699 | 1 00 | 0 86 |
| | ANRARNT_01 | KNNKNNTYGCGTGCMS | tccctgggCGTGtga | 10 150429 | 703 | 1 00 | 0 85 |
| | NFY_Q6 | TRRCCAATSRN | ctgCCAAIatt | 6 200634 | 730 | 1 00 | 0 87 |
| | CDP_01 | CCAATAATCGAT | ccAATAttcgtt | 147 430729 | 733 | 1 00 | 0 86 |
| | GATA_C | NGATAAGNMNN | tgctgTTATCt | 2 004465 | 744 | 1 00 | 0 93 |
| | GATA1_03 | NNNNNGATAANNGN | gctgtTATCttcgg | 2 776354 | 745 | 1 00 | 0 98 |
| | GFI1_01 | NNNNNNAAATCANNGNNNN | gggaaaggAATCcttgcclgggc | 3 689729 | 770 | 1 00 | 0 89 |
| | | NNNN | | | | | |
| | CP2_01 | GCNMNAMGMAG | CTCGgctgggc | 3 245733 | 787 | 1 00 | 0 90 |
| | RORA1_01 | NWAWNNAGGTCAN | ggctggGGTCag | 76 16064 | 807 | 1 00 | 0.85 |
| | AP1_Q2 | RSTGACTNMNW | tgpppGTCAgg | 5 905504 | 810 | 1 00 | 0 88 |
| | AP1FJ_Q2 | RSTGACTNMNW | tggggGTCAgg | 5 908504 | 810 | 1 00 | 0 91 |
| | NRF2_01 | ACCGGAAGNS | cctGGAAgag | 6 109080 | 522 | 1 00 | 0 86 |
| | E47_02 | NNNMRCAGGTGTTMNN | gcttcCCAGGtgaggct | 15 631450 | 832 | 1 00 | 0 86 |
| | ATF_01 | CNSTGACGTNNNYC | IggTGACpgaaagcg | 8 675151 | 860 | 1 00 | 0 92 |
| | CREB_Q4 | NSTGACGTMANN | ggTGACgaaagc | 16 981467 | 861 | 1 00 | 0 94 |
| | AP1_Q2 | RSTGACTNMNW | ggTGACgaaag | 9 018655 | 861 | 1 00 | 0 92 |
| | CREB_Q2 | NSTGACGTAANN | ggTGACgaaagc | 26 730221 | 861 | 1 00 | 0 95 |
| | APIFJ_Q2 | RSTGACTNMNW | ggTGACgaaag | 9 018855 | 861 | 1 00 | 0 95 |
| | CREBP1_Q2 | NSTGACGTMASN | ggTGACgaaagc | 22 127714 | 861 | 1 00 | 0 89 |
| | API_Q4 | RSTGACTMANN | ggTGACgaaag | 13 140586 | 861 | 1 00 | 0 91 |
| | CREB_01 | TGACGTMA | TGACgaaa | 4 176203 | 863 | 1 00 | 0 86 |
| | AP4_01 | WGARYCAGCTGYGGNCNK | aagtcccaGCTGtcagc | 178 524329 | 917 | 1 00 | 0 88 |
| | AP4_Q6 | CWCAGCTGGN | cccaGCTGtc | 18281794 | 927 | 1 00 | 0 97 |
| | AP4_Q6 | CWCAGCTGGN | ccCAGCtgtc | 18281794 | 922 | 1 00 | 0 94 |
| | AP4_Q5 | NNCAGCTGNN | cccaGCTGtcc | 2 628244 | 922 | 1 00 | 0 98 |
| | AP4_Q5 | NNCAGCTGNN | ccGAGClglc | 2 628244 | 922 | 1 00 | 0 96 |
| | AP1FJ_Q2 | RSTGACTNMNW | cagclGTCAgc | 5 905504 | 924 | 1 00 | 0 91 |
| | AP1_Q2 | RSTGACTNMNW | cagctGTCAgc | 5905504 | 924 | 1 00 | 0 89 |
| | GF1_01 | NNNNNNAAATCANNGNNNN | tggcagccAATCagatgcga | 3 660729 | 955 | 1 00 | 0 90 |
| | | NNNN | c | | | | |
| | CAAT_01 | NNNRRCCAATSA | ggcagCCAAtca | 4 415584 | 956 | 1 00 | 0 98 |
| | NFY_C | NCTGATTGGYTASY | ggcagCCAATcaga | 69 836703 | 956 | 1 00 | 0 96 |
| | NFY_Q6 | TRRCCAATSRN | cagCCAAtcag | 6 200634 | 958 | 1 00 | 0 96 |
| | AP4_Q6 | CWCAGCTGGN | gacgGCTGcg | 12 133646 | 976 | 1 00 | 0 86 |
| | AP2_Q6 | MKCCCSCNGGCG | cggctgCGGGtt | 7 064136 | 978 | 1 00 | 0 91 |
| | NFY_Q6 | TRRCCAATSRN | cccaTTGGttt | 6 200631 | 995 | 1 00 | 0 95 |
| | CAAT_01 | NNNRRCCAATSA | ccaTTGGtttac | 4 415584 | 996 | 1 00 | 0 91 |
| | TATA_01 | STATAAAWRNNNNNN | ggagcctcTTTAtcg | 7 166360 | 1025 | 1 00 | 0 86 |
| | GATA_C | NGATAAGNMNN | cctcITTATCg | 2 004465 | 1029 | 1 00 | 0 92 |
| | GATA1_03 | NNNNNGATAANNGN | dcttTATCgaglg | 2 776354 | 1030 | 1 00 | 0 93 |
| | AP1_Q2 | RSTGACTNMNW | agTGACtactg | 9018855 | 1040 | 1 00 | 0 93 |
| | AP1_Q4 | RSTGACTMANN | agTGACtactg | 13 148586 | 1040 | 1 00 | 0 93 |
| | AP1FJ_02 | RSTGACTNMNW | agTGAClaclg | 9 018855 | 1040 | 1 00 | 0 94 |
| | GFI1_01 | NNNNNNAAATCANNGNNNN | ctcgdctAATCagagrttagg | 3 600720 | 1056 | 1 00 | 0 94 |
| | | NNNN | | | | | |
| | STAT1_01 | NNNSANTTCCGGGMNTGN | cagagcttccaGGAAccctgc | 155 094175 | 1067 | 1 00 | 0 85 |
| | | SN | | | | | |
| | STAT_01 | TTCCCRKAA | TTCCaggaa | 6 281497 | 1073 | 1 00 | 0 95 |
| | STAT_01 | TTCCCRKAA | ttccaGGAA | 6281497 | 1073 | 1 00 | 0 97 |
| | GATA1_03 | NNNNNNGATAANNGN | IgIggGATAaaga | 2 776354 | 1090 | 1 00 | 0 95 |
| | GATA_C | NGATAAGNMNN | gGATAAaggaa | 2 004465 | 1094 | 1 00 | 0 94 |
| | BARBIE_01 | ATNNMAGCNCRNGCG | IcagAAAGgggcagg | 32 363969 | 1111 | 1 00 | 0 86 |
| | NFKB_Q6 | NGGGGAMTTTCCNN | caGGGAgttgcgcg | 26 126380 | 1122 | 1 00 | 0 88 |
| | NFKAPPAB_01 | GGGAMTTYCC | GGGAgtlgcc | 9 285691 | 1124 | 1 00 | 0 93 |
| | AP2_Q6 | MKCCCSCNGGCG | tgCCCGcagccg | 7 054136 | 1130 | 1 00 | 0 90 |
| | AP4_Q6 | CWCAGCTGGN | cgCAGCcgca | 12 133646 | 1134 | 1 00 | 0 86 |
| | XBP1_01 | NNGNTGACGTGKNNNWT | gcaccgcACGTcttcag | 21 302338 | 1141 | 1 00 | 0 85 |
| | VMYB_01 | AAYAACGCNN | gacCGTTgtc | 4 360548 | 1161 | 1 00 | 0 93 |
| | ER_Q6 | NNARGNNANNNTGACCYNN | gaccgttgtccTGACctct | 11 677290 | 1161 | 1 00 | 0 86 |
| | AP1FJ_Q2 | RSTGACTNMNW | ccTGACctctc | 2 792153 | 1170 | 1 00 | 0 90 |
| | RORA1_01 | NWAWNNAGGTCAN | ctGACCtctctgt | 7 616064 | 1171 | 1 00 | 0 92 |
| | NF1_Q6 | NNTTGGCNNNNNNCCNNN | aagagaaggtgGCCAaga | 1 651312 | −1093 | 1 00 | 0 94 |
| | DELTAEF1_01 | NNNCACCTNAN | agaAGGTggcc | 0 830664 | −1090 | 1 00 | 0 93 |
| Core | CMYB_01 | NNNNNNGNCNGTTGNN | ggccagagaGTTGgcgt | 0 187475 | −1083 | 1 00 | 0 86 |
| similarity >= | NF1_Q6 | NNTTGGCNNNNNNCCNNN | agtTGGCgtcatgagg | 1.651312 | −1074 | 1 00 | 0 91 |
| 0.99 | MZF1_01 | NGNGGGGA | tgtGGGGa | −0 225601 | −1035 | 1 00 | 0 99 |
| Template | IK2_01 | NNNYGGGAWNNN | tgtgGGGAgaga | −1 019855 | −1035 | 1 00 | 0 60 |
| similarity >= | M2F1_01 | NGNGGGGA | ttgGGGGa | −0 225601 | −1016 | 1 00 | 0 96 |
| 0.85 | 1K2_01 | NNNYGGGAWNNN | ttggGGGAtggg | 1 0119855 | −1016 | 1 00 | 0 89 |
| | MZF1_01 | NGN6GGGA | tggGGGGa | 0 225601 | −1008 | 1 00 | 0 98 |
| | IK2_01 | NNNYGGGAWNN | tgGGGAtcca | −1019855 | −1008 | 1 00 | 0 89 |
| | MZF1_01 | NGNGGGGA | cgaGGGGa | 0 225901 | −999 | 1 00 | 0 95 |
| | IK2_01 | NNNYGGGAWNNN | caagGGGA4cgat | −1 019655 | −999 | 1 00 | 0 91 |
| | DELTAEF1_01 | NNNCACCTNAN | gtccACCTcaa | 0 830664 | −987 | 1 00 | 0 96 |
| | 1K2_01 | NNNYGGGAWNNNN | cattGGAggag | −1019955 | 976 | 1 00 | 0 93 |
| | AP4_Q5 | NNCAGCTGNN | aaaaGCTGaa | 0 302731 | −960 | 1 00 | 0 88 |
| | MYOD_Q6 | NNCANCTGNY | cacGGGTGag | 0 740149 | −948 | 1 00 | 0 90 |
| | DELTAEF1_01 | NNNCACCTNAN | cacAGGTgcgt | 0 830604 | 948 | 1 00 | 0 97 |
| | IK2_01 | NNNVGGGAWNNN | ggccGGGActtg | −1019895 | −913 | 1 00 | 0 90 |
| | IK2_01 | NNNYGGGAWNN | tagaGGAgagg | 1019855 | −898 | 1 00 | 0 86 |
| | NF1_Q6 | NNTTGGCNNNNNNCGNNN | IgggrttctgGCGAttt | 1 651312 | −885 | 1 00 | 0 86 |
| | IK2_01 | NNNYGGGAWNNN | cagTCCCtcaa | −1 019855 | −857 | 1 00 | 0 91 |
| | NF1_Q6 | NNTTGGCNNNIINNCCNNN | cttTGGCtgcactcacc | 1 651312 | 831 | 1 00 | 0 93 |
| | DELTAEF1_01 | NNNCACCTNAN | ctctACCTtac | 0 830664 | −820 | 1 00 | 0 88 |
| | NMYC_01 | NNNCACGTGNNN | agtctCGTGgcc | 0 303606 | 803 | 1 00 | 0 88 |
| | CMYB_01 | NNNNNNGNCNGTTGNN | atgtctccccGTTGgrga | 0 187475 | −782 | 1 00 | 0 90 |
| | IK2_01 | NNNYGGGAWTNNN | tgtcTCCCcgtt | −1019856 | −781 | 1 00 | 0 88 |
| | MZF1_01 | NGNGGGGA | ICCCCgtt | −0 229801 | −777 | 1 00 | 0 96 |
| | VMYB_02 | NSVAACGGN | ccCGTTggc | 0 427098 | −775 | 1 00 | 0 96 |
| | NF1_Q6 | NNTTGGCNNNNNNCCNNN | cgTGGCgaactcctctt | 1 651312 | −773 | 1 00 | 0 94 |
| | GATA1_04 | NNCWGATARNNNN | ctattTATCccta | 1 126824 | −760 | 1 00 | 0 92 |
| | GATA1_02 | NNNNNGATANKGNN | ctcTTATCctcaa | 1 132907 | −760 | 1 00 | 0 89 |
| | LMO2COM_02 | NMGATANSG | attTATCct | 0 679503 | −758 | 1 00 | 0 88 |
| | CMYB_01 | NNNNNNGNCNGTTGNN | gtccCAACggctgcca | 0 187475 | −746 | 1 00 | 0 94 |
| | VMYB_02 | NSYAACGGN | cccAACGgc | 0 327098 | −743 | 1 00 | 0 99 |
| | DELTAEF1_01 | NNNCACCTNAN | IgcACCTcct | 0 830664 | −732 | 1 00 | 0 93 |
| | IK2_01 | NNNYGGGAWNNN | ccgcGGGAgccg | 1 019955 | −720 | 1 00 | 0 89 |
| | IK2_01 | NNNYGGGAWNNN | gcrgTCCCcacg | −1 019955 | −712 | 1 00 | 0 91 |
| | MZF1_01 | NGGGGA | IKCCCCarg | 0 667940 | 708 | 1 00 | 0 99 |
| | IK2_01 | NNNYGGAWNNN | actctCCCagc | −1 019955 | 694 | 1 00 | 0 88 |
| | MZF1_01 | NGNGGGA | tCCCCagc | 0 687940 | −690 | 1 00 | 0 97 |
| | AP4_Q5 | NNCAGCTGNN | ccCAGCgcct | 0 302731 | −688 | 1 00 | 0 86 |
| | AP4_Q5 | NNCAGCTGNN | caaaGCTtg | 1465487 | 677 | 1 00 | 0 90 |
| | IK2_01 | NNNYGGGAWNNN | acgcTCCCatt | −1019855 | −660 | 1 00 | 0 91 |
| | AP4_Q5 | NNGAGCTGNN | ttCAGCttca | 0 302731 | −660 | 1 00 | 0 87 |
| | DELTAEF1_01 | NNNCACCTNAN | cttcACCTCcca | 0 830684 | −645 | 1 00 | 0 95 |
| | IK2_01 | NNNYGGGAWNNN | gagtGGAaacg | 1 019855 | −603 | 1 00 | 0 96 |
| | VMYB_02 | NSYAACGGN | ggaAACGgg | 0 327098 | −958 | 1 00 | 0 90 |
| | 88_01 | NNNNNYAATTN | actaTAATcggagact | −0 676908 | −586 | 1 00 | 0 86 |
| | NF1_Q6 | NNTTGGCNNNNNNCCNNNN | IgTGGCcccctcccct | 1 651312 | −544 | 1 00 | 0 95 |
| | IK2_01 | NNNYGGGAWNNN | ccccTCGCcctc | −1019855 | 537 | 1 00 | 0 87 |
| | MZF1_01 | NGNGGGGA | tCCCCctr | 0 225601 | −531 | 1 00 | 0 96 |
| | IK2_01 | NNNYGGGAWNN | gtagTCCCagag | −1010865 | −434 | 1 00 | 0 96 |
| | 1K2_01 | NNNYGGGAWNN | tagaGGGAgcct | −1 019055 | −410 | 1 00 | 0 88 |
| | NF1_Q6 | NNTTGGCNNNNNNCCNNN | gaggagcctgGCCAgtc | 1 851312 | −408 | 1 00 | 0 88 |
| | MZF1_01 | NGNGGGGA | cccGGGGa | 0 225601 | 391 | 1 00 | 0 95 |
| | IK2_01 | NNINYGGGAWNNN | cccgGGGAcagc | 1 010855 | 391 | 1 00 | 0 89 |
| | AP4_Q5 | NNCAGCTGNN | gaCAGCgggg | 1 465487 | −385 | 1 00 | 0 92 |
| | IK2_01 | NNNYGGGAWNNN | agcgGGGAcaga | −1 010855 | −382 | 1 00 | 0 88 |
| | MZF1_01 | NGNGGGGA | agcGGGGa | −0 225601 | −382 | 1 00 | 0 99 |
| | IK2_01 | NNNYGGCAWNNN | cagaGGGAactc | −1 019855 | −374 | 1 00 | 0 94 |
| | CEBPB_01 | RNRTKNNGMAAKNN | aactcctGGAAttc | 1 857489 | −367 | 1 00 | 0 88 |
| | AP4_Q5 | NNCAGCTGNN | tgCAGCcggt | 1 465487 | −340 | 1 00 | 0 90 |
| | ARNT_01 | NNNNNCACGTGNNNNN | tatacaaCGTGgggag | 0 305357 | −330 | 1 00 | 0 88 |
| | MZF1_01 | NGNGGGGA | cgtGGCGa | 0 607040 | −323 | 1 00 | 0 99 |
| | IK2_01 | NNNYGGCAWNNN | cgtgGGGAggca | −1 019855 | −323 | 1 00 | 0 88 |
| | IK2_01 | NNNYGGGAWNNN | lggcTGCCcaaa | −1 019855 | −308 | 1 00 | 0 89 |
| | MZF1_01 | NGNGGGGA | tCCCCaaa | −0 225601 | −304 | 1 00 | 0 96 |
| | AP4_Q5 | NNCAGCTGNN | gaCAGCgcag | 0 302731 | −296 | 1 00 | 0 86 |
| | IK2_01 | NNNYGGGAWNNN | tcgttCCCggag | −1 019855 | −284 | 1 00 | 0 94 |
| | CETS1P54_01 | NCMGGAWGYN | ccCGGApggc | 1 032772 | 279 | 1 00 | 0 89 |
| | IK2_01 | NNNYGGGAWNNN | gccIGGGActg | −1 019855 | −264 | 1 00 | 0 92 |
| | NF1_06 | NNTTGGCNNNNCCNNNCNNN | ccgggcactacGCCAccc | 1 651312 | −252 | 1 00 | 0 87 |
| | CEBPB_01 | RNRTKNNGMAAKNN | tgatcaGCAAgag | 1 857489 | 229 | 1 00 | 0 90 |
| | IK2_01 | NNNYGGGAWNNN | gcaggTCCCtta | −1 010855 | 211 | 1 00 | 0 91 |
| | IK2_01 | NNNYGGGAWNNN | gtgaTCCCgtct | −1 018855 | −172 | 1 00 | 0.93 |
| | IK2_01 | NNNYGGGAWNNN | clccrCCCltgg | −1 019855 | −162 | 1 00 | 0 88 |
| | NF1_06 | NNTTGGCNNNNNNCCNNN | ccTGGCccgcgcagctc | 1 651312 | −156 | 1 00 | 0 92 |
| | NF1_06 | NNTTGGCNNNNNNCCNNN | cgacggagcagGCCAgtg | 1 651312 | −138 | 1 00 | 0 86 |
| | CREB_02 | NNGNTGACGYNN | tgagTGACgggc | 0 972541 | −122 | 1 00 | 0 88 |
| | AP4_Q5 | NNGAGCTGNN | cggcGCTGct | 0 302731 | −70 | 1 00 | 0 88 |
| | DELTAEF1_01 | NNNCACCTNAN | tgttACCTgcg | 0 830604 | −64 | 1 00 | 0 85 |
| | AP4AP4 Q5 | NNCAGCCTGNN | ctCAGCgac | 0 302731 | −45 | 1 00 | 0 87 |
| | NKX25_05 | TYAAGTG | cACTTgg | 1 905547 | −38 | 1 00 | 0 93 |
| | NF1_Q6 | NNTTGGCNNNNNNCCNNN | actTGGCttaaggggcgg | 1 651312 | −37 | 1 00 | 0 92 |
| | IK2_01 | NNNYGGGAWNNN | gcgcTCCCTgcc | −1 010855 | −18 | 1 00 | 0 90 |
| | AP4_Q5 | NNCAGCTGNN | gcctGCTGct | 1 405487 | −9 | 1 00 | 0.92 |
| | AP4_Q5 | NNCAGCTGNN | IgctGCTGyy | 0 302731 | −6 | 1 00 | 0 90 |
| | IK2_01 | NNNYGGGAWNNN | cggnGGAAaggc | −1 019865 | 4 | 1 00 | 0 83 |
| | AP4_Q5 | NNCAGCIGNN | aagaGCTGag | 1 465487 | 19 | 1 00 | 0 93 |
| | DELTAEF1_01 | NNNCACCTNAN | ggaAGGTtgaga | 0 830864 | 37 | 1 00 | 0.97 |
| | IK2_01 | NNNYCGGAVWNNN | gagaGGGAagaa | −1 019855 | 58 | 1 00 | 0 93 |
| | IK2_01 | NNNYGGGAWNNN | ggccGGGAggga | −1 019855 | 96 | 1 00 | 0 90 |
| | IK2_01 | NNNYGGGAWNNN | gggaGGGAtgca | −1 019855 | 100 | 1 00 | 0 91 |
| | IK2_01 | NNNYGGGAWNNN | aagtTCCGtggg | −1 019855 | 111 | 1 00 | 0 91 |
| | SB_01 | NNNNNYAATTN | ccctgggaATTAgggg | −0 676808 | 116 | 1 00 | 0 93 |
| | IK2_01 | NNNYGGGAWNNN | ccctGGGAatta | −1 019855 | 116 | 1 00 | 0 96 |
| | CETSIP54 _01 | NCMGGAWGYN | tcctTCCGgt | 1 032772 | 147 | 1 00 | 0 95 |
| | CMYB_01 | NNNNNNNGNCNGTTGNN | tccggtgaatGTTGacga | 0 187475 | 151 | 1 00 | 0 85 |
| | CREB_02 | NNGNTGACGYNN | atgTGACgacg | 0 972541 | 159 | 1 00 | 0 93 |
| | AP4_Q5 | NNCAGCTGNN | gacgGCTGaa | 0 302731 | 167 | 1 00 | 0 89 |
| | IK2_01 | NWNYGGGAWNNN | atctGGAacct | −1 1019855 | 209 | 1 00 | 0 93 |
| | DELTAEF1_01 | NNNCACCTNAN | acacACCTgca | 0 836061 | 241 | 1 00 | 0 96 |
| | MYOD_Q6 | NNCANCTGNY | caCACCIgra | 0 175805 | 242 | 1 00 | 0 91 |
| | VMYB_02 | NSYAACGGN | ccCGTTaga | 0 327098 | 260 | 1 00 | 0 96 |
| | IK2_01 | NNNYGGGAWNNN | gcacTCCCcctt | −1 019855 | 275 | 1 00 | 0 90 |
| | MZF1_01 | NGNGGGGA | ICCCCctt | −0 225801 | 279 | 1 00 | 0 97 |
| | IK2_01 | NNNYGGGAWNNN | ccacTCCCccag | −1 019855 | 316 | 1 00 | 0 88 |
| | MZF1_01 | NGNGGGGA | IGCCCcag | −0 225601 | 320 | 1 00 | 0 96 |
| | IK2_01 | NNNYGGGAWNNNN | taagTCCCgctt | −1 019855 | 332 | 1 00 | 0 91 |
| | IK2_01 | NNNYGGGAWNNN | gaggTCCCagtt | −1 019855 | 383 | 1 00 | 0 94 |
| | CETSIP54_01 | NCMGGAWGYN | cagtTCCGgc | 1032772 | 390 | 1 00 | 0 93 |
| | DELTAEF1_01 | NNNCACCTNAN | ggtcACCTtta | 0 830664 | 402 | 1 00 | 0 95 |
| | CEBPB_01 | RNRTKNNGMAAKNN | acctttaGCAActt | 1 857489 | 406 | 1 00 | 0 93 |
| | DELTAEF1_01 | NNNCACCTNAN | cagAGTggac | 0 830664 | 457 | 1 00 | 0.94 |
| | GATA1_02 | NNNNNGATANKGNN | gtctcTATCccagc | 1132007 | 508 | 1 00 | 0 92 |
| | GATA1_04 | NNCWGATARNNNN | gtctgTATCccag | 1128024 | 508 | 1 00 | 0 90 |
| | LMO2COM_02 | NMGATANSG | ctcTATCcc | 0 670593 | 510 | 1 00 | 0 93 |
| | IK2_01 | NNNYGGGAWNNN | tctaTCCCagcc | −1010855 | 511 | 1 00 | 0 95 |
| | IK2_01 | NNNYGGGAWNNN | tggcTCCCgcgg | −1 010855 | 543 | 1 00 | 0 89 |
| | IK2_01 | NNNYGGGAWNNN | gcggTCCCtctg | −1 019855 | 551 | 1 00 | 0 91 |
| | SB_01 | NNNNNYAATTN | tctgagcgATTAtgc | −0 676808 | 559 | 1 00 | 0 86 |
| | DELTAEF1_01 | NNNCACCTNAN | ataGGTgtgg | 0 830884 | 578 | 1 00 | 0.97 |
| | AP4_Q5 | NNCAGCTGNN | cagaGCTGgg | 1 465487 | 590 | 1 00 | 0.91 |
| | CMYB_01 | NNNNNNGNCNCTTGNN | tgggCAACtgcctgtctc | 0 187475 | 614 | 1 00 | 0 94 |
| | NKX25_01 | TYAAGTG | CACTTct | 1 905547 | 670 | 1 00 | 0 88 |
| | GATA1_02 | NNNNNGATANKGNN | ggggtGATAtcca | 1 132907 | 604 | 1 00 | 0 93 |
| | GAT1_04 | NNCWGATARNNNN | gggIGATAgtcca | 1 128924 | 685 | 1 00 | 0.92 |
| | LMO2COM_02 | NMGATANSG | gtGATAgtc | 0 679593 | 687 | 1 00 | 0 92 |
| | IK2_01 | NNNYGGGGAWNNN | cactTCCCCtgg | −1 010855 | 699 | 1 00 | 0 89 |
| | NKX25_01 | TYAAGTG | cACTTcc | 1 905547 | 690 | 1 00 | 0 88 |
| | MZF1_01 | NGNGGGGA | ICCCCIgg | −0 223601 | 701 | 1 00 | 0 95 |
| | NFl_Q6 | NNTTGGCNNNNNNCCNNN | tgtccagacatGCCAata | 1 051312 | 721 | 1 00 | 0 92 |
| | GATA1_04 | NNCWGATARNNNN | gctgtTATCttcg | 1 128924 | 745 | 1 00 | 0 95 |
| | GTA1_02 | NNNNNGATANKGNN | gctgtTATCttrgg | 1 132907 | 745 | 1 00 | 0 92 |
| | LMO2COM_02 | NMGATANSG | tgtTATCtt | 0 679583 | 747 | 1 00 | 0 94 |
| | MZF1_01 | NGNGGGGA | IgaGGGGa | −0 225001 | 706 | 1 00 | 0 97 |
| | IK2_01 | NNNYGGGAWNNN | tgagGGGAaagg | −1 019855 | 766 | 1 00 | 0 90 |
| | NF1_Q6 | NNTTGGCNNNNNNCCNNN | gcctgggctggGCCAggc | 1 651312 | 785 | 1 00 | 0 85 |
| | LMOC0M_01 | SNNCAGGTGNNN | ttcCAGGtgagg | 0 773414 | 834 | 1 00 | 0 94 |
| | DELTAEF1_01 | NNNCACCTNAN | tccAGGTgagg | 0 830664 | 835 | 1 00 | 0 98 |
| | MYOD_Q8 | NNCANCTGNY | tccaGGTGag | 0 740149 | 835 | 1 00 | 0 90 |
| | CREB_02 | NNGNTGACGYNN | ctggTCACgaaa | 0 972541 | 8519 | 1 00 | 0 94 |
| | IK2_01 | NNNYGGGAWNNN | tcggTCCCtggca | −1 019855 | 886 | 1 00 | 0 89 |
| | NKX25_01 | TYAAGTG | ttAAGTc | 1 905547 | 915 | 1 00 | 0 86 |
| | IK2_01 | NNNYGGGAWNNN | taagTCCCcagc | 1 019855 | 916 | 1 00 | 0 90 |
| | MZF1_01 | NGNGGGGGA | tGCCCagc | 0 667940 | 920 | 1 00 | 0 97 |
| | AP4_Q5 | NNCAGCTGNN | gtCAGCCCcctg | 0 302731 | 929 | 1 00 | 0 66 |
| | NF1_Q6 | NNTTGGCNNNNNNCCNNN | cagtcctggcaGCCAatc | 1 651312 | 949 | 1 00 | 0 92 |
| | NF1_Q6 | NNTTGGCNNNNNNCCNNN | tccTGGCagccaatcaga | 1 651312 | 952 | 1 00 | 0 89 |
| | AP4_Q5 | NNCAGCTGNN | gacgGCTGcg | 1 465487 | 976 | 1 00 | 0 91 |
| | IK2_01 | NNNYGGGAWNNN | gcgcTCCCattg | −1 019855 | 990 | 1 00 | 0 94 |
| | GATA1_04 | NNCWGATARNNNN | ctcttTATCgagt | 1 120924 | 1030 | 1 00 | 0 92 |
| | GATA1_02 | NNNNNGATANKGNN | ctcttTATCgagtg | 1 132907 | 1030 | 1 00 | 0 92 |
| | LMO2COM_02 | NMGATANSG | ctTATCga | 0 679593 | 1032 | 1 00 | 0.95 |
| | S8_01 | NNNNNYAATTN | ActcTATcagagctt | −0 678808 | 1059 | 1 00 | 0 85 |
| | AP4_Q5 | NNCAGCTGNN | ctgcGCTGtg | 0 302731 | 1064 | 1 00 | 0.87 |
| | IK2_01 | NNNYGGGAWNNN | ctgtGGGAtaaa | 1 010855 | 1 009 | 1 00 | 0.95 |
| | GATA1_02 | NNNNNGATANKGNN | tgtggGAIAaagga | 1 132907 | 1090 | 1 00 | 0.93 |
| | GATA1_04 | NNCWGATARNNNN | gtggGATAaagga | 1 128924 | 1091 | 1 00 | 0 93 |
| | LMO2COM_02 | NMGATANSG | ggGATAaag | 0 679593 | 1093 | 1 00 | 0.93 |
| | CMYB_01 | NNNNNNGNCNGTTGNN | ggggcagggaGTTGcccg | 0 187475 | 1118 | 1 00 | 0.88 |
| | IK2_01 | NNNYGGGAWNNN | ggcaGGGAgttg | −1 019855 | 1120 | 1 00 | 0 89 |
| | AP4_Q5 | NNCAGCTONN | cgCAGCcgca | 1 465487 | 1134 | 1 00 | 0 90 |
| | ARNT_01 | NNNNNCACGTGNNNNN | caccgCACGtcttcag | 0 305357 | 1142 | 1 00 | 0 86 |
| | CMYB_01 | NNNNNNGNCNGTTGNN | cagcccgaccGTTGtcct | 0 187475 | 1155 | 1 00 | 0 93 |
| | VMYB_02 | NSYAACGGN | acGTTgtc | 0 327098 | 1162 | 1 00 | 0 97 |
| | IK2_01 | NNNYGGGAWNN | tctgTCCCgtcc | −1 010855 | 1179 | 1 00 | 0.91 |
| | IK2_01 | NNNYGGGAWNNN | ccgTCCCctgc | −1 019855 | 1184 | 1 00 | 0 87 |
| | MZF1_01 | NGNGGGGA | ICCCCtgc | −0 225601 | 1188 | 1 00 | 0 95 |
| | HNF3B_01 | NNNTRTTTRYTY | ctcTGTTtgtac | 3 978804 | −1106 | 0 99 | 0 84 |
| None | CDPCR3HD_01 | NATYGATSSS | cgtcGATGag | 2 474120 | −1068 | 0 93 | 0 85 |
| (MatIspector | USF_Q6 | GYCACGTGAC | gcCACAggtg | 5 390268 | −950 | 0 88 | 0 87 |
| default | E47_01 | NSNGCAGGTGKCNN | gccACAGgtgagtct | 6 708124 | −950 | 0 83 | 0 86 |
| parameters) | USF_Q6 | GYCACGTGNC | cacaGGTCag | 10 960075 | −948 | 0 82 | 0 87 |
| | USF_C | NACACTGTN | acAGGTCa | 0 301857 | −947 | 0 86 | 0 92 |
| | CETS1P54_01 | NCMGGAWGYN | ggctCCTgg | 1 032772 | 883 | 0 93 | 0.95 |
| | CAAT_01 | NNRRCCAATSA | cctggCCATttg | 4 415584 | −878 | 0 86 | 0 86 |
| | CEBPB_01 | RNRTKNNGMAAKNN | cagTTCCctcaaat | 1 857489 | −857 | 0 87 | 0 86 |
| | AP2_Q6 | MAKCCCSCNGGCG | gcCCCCcatgcg | 7 064136 | −843 | 0 98 | 0 86 |
| | USF_C | NCACGTGN | ITCGTgg | 0 301857 | −801 | 0 81 | 0 86 |
| | CETS1P54_01 | NCMGGAWGYN | ccTGGAtgtc | 1 032772 | 787 | 0 85 | 0 92 |
| | CETS1P54_01 | NCMGGAWGYN | caccTCCTgc | 1 032772 | −729 | 0 93 | 0 92 |
| | CETS1P54_01 | NCMGGAWGYN | caccTCCAgc | 1 032772 | −642 | 0 85 | 0 89 |
| | CETS1P54_01 | NCMGGAWGYN | ttctTCCAga | 1 032772 | −611 | 0 85 | 0 90 |
| | CEBPB_01 | RNRTKNNGNAAKNN | ggtTTCctcaaaa | 1 857489 | −591 | 0 99 | 0 88 |
| | CEBPB_01 | RNRTKNNGNAAKNN | gttTTCCcaaaat | 1 857489 | −590 | 0 87 | 0 90 |
| | GC_01 | NRGGGGCGGGGCNK | ggccccCTCCccct | 15 933816 | −540 | 0 88 | 0 91 |
| | SP1_Q6 | NGGGGCGGGGYN | gccccCTCCcccct | 11 119144 | −539 | 0 84 | 0 93 |
| | CETS1P54_01 | NCMGGAWGYN | ccccTCCTgc | 1 032772 | −531 | 0 93 | 0 87 |
| | AP2_Q6 | MKCCCSCNGGCG | agCCCCggggac | 7 064136 | −394 | 0 98 | 0 86 |
| | AP2_Q6 | MKCCCSCNGGCG | agccccGGGac | 7 064136 | −394 | 0 98 | 0 88 |
| | RFX1_02 | NNGTNRCNNNRGTAACNN | cggggcacgagGGAActc | 7 228454 | −380 | 0 88 | 0 90 |
| | NFKAPPAB85_01 | CGGRATTTCC | GGGAactcct | 14 122479 | −370 | 0 83 | 0 69 |
| | CETS1P54_01 | NCMGGAWGYN | gaacTCCTgc | 2 674616 | −368 | 0 93 | 0 85 |
| | VMYB_01 | AAYAACGGNN | gccGGTTata | 4 365048 | −336 | 0 81 | 0 86 |
| | TATA_01 | STATAAAWRNNNNNN | ttaTACAacgtgggg | 7 166360 | −331 | 0 80 | 0 86 |
| | LYF1_01 | TTTCGGAGR | ctCCCaaa | 7 208594 | −305 | 0 82 | 0 85 |
| | CEBPB_01 | RNRTKNNGMAAKNN | ccttaaGAAAccc | 1 857489 | −205 | 0.99 | 0 89 |
| | PAD5_C | NGTGGCTC | IGTGATccc | 4 266174 | −173 | 0 90 | 0 86 |
| | CAAT_01 | NNNRRCCAATSA | gcaggCCAGga | 4 416584 | −131 | 0 85 | 0 90 |
| | USF_Q6 | GYCACGTGNC | ggccaACTGag | 5 390268 | −128 | 0 86 | 0 89 |
| | AP1_C | NTGASTCAN | gTGAGTGac | 1 751881 | −123 | 0 85 | 0 87 |
| | AP1_C | NTGASTCAN | gTGAGTGac | 1 751881 | −123 | 0 86 | 0 86 |
| | NFKAPPAB_01 | GGGAMTTYCC | GGGGcgtgcc | 9 285691 | −81 | 0 90 | 0 87 |
| | USF_Q6 | GTCACGTGNC | cgCACTggc | 5 390268 | −40 | 0 86 | 0 89 |
| | USF_C | NCACGTGN | gCACTTgg | 0 301857 | −39 | 0.84 | 0 91 |
| | CETS1P54_01 | NCMGGAWGYN | ccTGGAaggt | 1 032772 | 34 | 0 85 | 0 88 |
| | RFX1_01 | NNGTNRCNNRGTAACNN | aagTTCCctgggaatta | 7 228828 | 111 | 0 88 | 0 89 |
| | CLOX_01 | NNTATCGATTANYNW | tgaATTGatcactga | 81 979826 | 173 | 0 87 | 0 89 |
| | CDP_02 | NNATCGATTANYNN | tgaATTGatcactga | 37 346724 | 173 | 0 85 | 0 89 |
| | LMO2COM_01 | SNNCAGGTGNNN | ggacaTCTGgga | 0 773414 | 205 | 0 82 | 0 90 |
| | MYOD_Q6 | NNCANCTGNY | gaCATCtggg | 0 175805 | 206 | 0 92 | 0 89 |
| | USF_C | NCACGTCN | aCACCTgc | 0 301857 | 243 | 0 86 | 0 92 |
| | AP2_Q6 | MKCCCSCNGGCG | agCCCCctgccc | 7064136 | 201 | 0 96 | 0 88 |
| | CMYB_01 | NNNNNNGNCNGTTGNN | ccccctgcccGTTAgaac | 0 187475 | 253 | 0 84 | 0 85 |
| | CETS1P54_01 | NCMGGAWGYN | gaacTCCTgc | 2 674615 | 267 | 0 93 | 0 85 |
| | AP2_Q6 | MKCGCSCNGGCG | ctCCCCCctgcc | 7 064136 | 278 | 0 98 | 0.88 |
| | CEBPB_01 | RNRTKNNGMAAKNN | aaatggaGAAActg | 1 857489 | 299 | 0 99 | 0 92 |
| | VMYB_01 | AAYAAGGGNN | agaAACTgag | 4 360548 | 305 | 0 88 | 0 87 |
| | CEBPD_01 | RNRTKNNGMAAKNN | gcttgatGTAAagg | 1 857489 | 340 | 0 93 | 0 89 |
| | CEDP8_01 | RNRTKNNGMAAKNN | atgtaaaGGAAaga | 1 857489 | 345 | 0 87 | 0 86 |
| | CEBPB_01 | RNRTKNNGMMKNN | ccctggcGTAAggg | 1 857489 | 360 | 0 93 | 0 88 |
| | CETS1P54_01 | NCMGGAWGYN | aactTCCTgc | 1 032772 | 416 | 0 93 | 0 96 |
| | LMO2COM_01 | SNNCAGGTGNNN | ctccaTGTGtgt | 0 773414 | 445 | 0 82 | 0.90 |
| | MYOD_Q6 | NNCANCTGNY | tcCATCtgtg | −0 175805 | 446 | 0 92 | 0.91 |
| | AP1_C | NTGASTCAN | cTGTGTCAg | 1 751681 | 451 | 0 86 | 0 88 |
| | CLOX_01 | NNTATCGATTANYNW | aaaATAGatcaggaa | 81 978936 | 478 | 0 81 | 0 85 |
| | CDP_02 | NWNATCGATTANYNN | aaaATAGatcaggaa | 37 346724 | 478 | 0 81 | 0 88 |
| | CETS1P54_01 | NCMGGAWGYN | tcgAGGAatcg | 1 032772 | 486 | 0 93 | 0 88 |
| | GATA_C | NGATMGNMNN | agtctCTATCc | 2 004465 | 507 | 0 89 | 0 92 |
| | GC_01 | NRGGGGCGGGGCNK | IgtgGGCAgagctg | 15 933816 | 584 | 0 81 | 0 86 |
| | CETS1P54_01 | NCMGGAWGYN | tgccTCCAgc | 1 032772 | 604 | 0 85 | 0.89 |
| | LMO200M_01 | SNNCAGGTGNNN | ctccaGCTGggc | 0 773414 | 607 | 0 88 | 0 94 |
| | LMO2COM_01 | SNNCAGGTGNNN | ctcCAGCIgggc | 0 773414 | 607 | 0 88 | 0 93 |
| | MYOD_Q6 | NNCANCTGNY | tcCAGCIggg | 1 656102 | 608 | 0 92 | 0 90 |
| | MYOD_Q6 | NNCANCTGNY | tccaGCTGgg | 1 656102 | 608 | 0 92 | 0 90 |
| | LMO2COM_01 | SNNCAGGTGNNN | gggcaACTGcct | 0 773414 | 815 | 0 80 | 0.91 |
| | VMYB_01 | MYAACCGNN | ggcAACTgcc | 4.360548 | 616 | 0.88 | 0.86 |
| | VMYB_02 | NSYAACGGN | ggcAACTgc | 0.327098 | 616 | 0 82 | 0 89 |
| | MYOD_Q6 | NNCANCTGNY | ggCAACtgcc | −0 175805 | 616 | 0.87 | 0.97 |
| | GATA_C | NGATAAGNMNN | IGATAGtccag | 2 004465 | 688 | 0 89 | 0 88 |
| | AP2_Q6 | MKCCCSCNGGCG | ttCCCCtgggcg | 7 064136 | 702 | 0 98 | 0 88 |
| | USF_Q6 | GYCACGTGNC | ggcgTGTGaa | 5 390288 | 710 | 0 86 | 0.87 |
| | CHOP_01 | NNRTGCAATMCCC | gtgTGAAagtcc | 22326380 | 713 | 0 80 | 0.86 |
| | CETS1P54_01 | NCMGGAWGYN | aatgTCCAgc | 1 032772 | 719 | 0 65 | 0 86 |
| | OCT1_02 | NNGAATATKCANNNN | gccaatATCgttgc | 11 865447 | 732 | 0 98 | 0 91 |
| | COPCR3_01 | CACCRATANNTATNG | CAATattcgttgctg | 92 376068 | 734 | 0 97 | 0 86 |
| | VMYB_01 | AAYAACGGNN | IgcTGTTatc | 4 360548 | 744 | 0 82 | 0 89 |
| | STAT_01 | TTCCCRKAA | ttcggAGAA | 6 281497 | 754 | 0 81 | 0 88 |
| | CETS1P54_01 | NCMGGAWGYN | gcACGAggct | 1 032772 | 801 | 0 93 | 0 90 |
| | AP2_Q6 | MKCCCSCNGGGCG | aggctgGGGt | 7 064136 | 806 | 0 98 | 0 85 |
| | CETS1P54_01 | NCMGGAWGYN | tcAGGAcctg | 1 032772 | 816 | 0 93 | 0 87 |
| | CETS1P54_01 | NCMGGAWGYN | ccTGGAagag | 1 032772 | 822 | 0 85 | 0 89 |
| | CETS1P54_01 | NCMGGAWGYN | gggctCCAgg | 1 032772 | 831 | 0 85 | 0 92 |
| | USF_Q6 | GYCACGTGNC | tccaGGTGag | 10 960175 | 835 | 0 82 | 0.86 |
| | USF_C | NCACGTGN | ccAGGTGa | 0 301857 | 836 | 0 86 | 0 92 |
| | SP1_Q6 | NGGGGGCGGGGYN | ttggGGTGgagcc | 11 119144 | 847 | 0 82 | 0 87 |
| | GC_01 | NRGGGGCGGGGCNK | ttggGGTGgagcct | 15 933816 | 847 | 0 87 | 0 91 |
| | USF_Q6 | GYCACGTGNC | gcctGGTGac | 5 390288 | 857 | 0 82 | 0 90 |
| | CEBPB_01 | RNRTKNNGMAAKNN | tggtgacCAAAgcg | 1 857489 | 850 | 0 99 | 0 91 |
| | MYOD_01 | SRACAGGTGKYG | cccaGCTGtca | 32 905282 | 621 | 0 83 | 0 86 |
| | LMO2COM_01 | SNNCAGGTGNNN | cacaGCTGtca | 2 232288 | 921 | 0 88 | 0 92 |
| | LMO2COM_01 | SNNCAGGTGNNN | cccCAGCgtca | 0 773414 | 921 | 0 88 | 0.93 |
| | MYOD_Q6 | NNCANCTGNY | CCCAGCtgtc | 1 656102 | 922 | 0 92 | 0 98 |
| | MYOD_Q6 | NNCANCTGNY | cccaGCTGtc | 1 655102 | 922 | 0 92 | 0 89 |
| | LMO2COM_01 | SNNCAGGTGNNN | aatCAGAtgcga | 0.773414 | 963 | 0 82 | 0 89 |
| | MYOD_Q6 | NNCANCTGNY | atcaGATGcg | −0.175805 | 964 | 0 92 | 0 94 |
| | CEBPB_01 | RNRTKNNGMAAKNN | ggITTACIccaccc | 1 857489 | 1001 | 0 93 | 0 90 |
| | GC_01 | NRGGGGCGGGGCNK | ttactcCACCcctg | 15 933816 | 1004 | 0 87 | 0 87 |
| | SP1_Q6 | NGGGGGCGGGGYN | tactcCACCcctg | 11 119144 | 1005 | 0 82 | 0 85 |
| | USF_Q6 | GYCACGTGNC | atcgAGTGac | 5 390268 | 1036 | 0 86 | 0 88 |
| | HNF3B_01 | NNNTRTTTRYTY | tacTGTTtgcct | 3 978804 | 1046 | 0 99 | 0 92 |
| | CETS1P54_01 | NCMGGAWGYN | agctTCCAgg | 1 032772 | 1070 | 0 85 | 0 92 |
| | CETS1P54_01 | NCMGGAWGVN | ccAGGAaccc | 1 032772 | 1075 | 0 93 | 0 89 |
| | CEBPB_01 | RNRTKNNGMAAKNN | gggataaaGCAAtga | 1 857489 | 1094 | 0 87 | 0 89 |
| | CEBPB_01 | RNRTKNNGMAAKNN | agttcaGAAAggg | 1 857489 | 1107 | 0 99 | 0 94 |
| | GC_01 | NRGGGGCGGGGCNK | aggGCCAgggagt | 15 933816 | 1116 | 0 81 | 0 85 |
| | NFKB_C | NGCGACTTTCCA | aGGGAGttgccc | 42 31372 | 1123 | 0 88 | 0.90 |

TABLE 2
Sites, scores, consensus and positions relative to the site of initiation of
transcription (TSS) prectictect by the NNPP, TSSG anct TSSW software packages in mice

| | | | | | | Core | |
|---|---|---|---|---|---|---|---|
| | | | | | Position/ | simi- | Template |
| Filtration | Site | Consensus | Secquence | Z score | TSS(bp) | larity | similarity |
| Comparative | GFI1_01 | NNN | ttgcctacAATCaggcaactatt | 2 393233 | −842 | 1 00 | 0 66 |
| analysis | | NNOMNNAAATCANNGNNNN | | | | | |
| between | HNF3B_01 | NNNTRTTTRYTY | aacTATTgattc | 2 929849 | −825 | 1 00 | 0 85 |
| species | CEPB_01 | RNHTKNNGMAAKNN | tgattctGAAAttg | 1 460836 | −787 | 0 99 | 0 94 |
| | CEBPB_01 | RNRTKNGMAAKNN | atgTTGCtaaatg | 1 460836 | −760 | 1 00 | 0 91 |
| | NF1_Q6 | NNTTGGCNNNNNWCCNN | ttcTGGCtggtggcagga | 2 199282 | −668 | 1 00 | 0 88 |
| | AP4_Q6 | CWCAGCTGGN | caCAGCgtg | 14 114395 | −386 | 1 00 | 0 87 |
| | NFKAPPAB_01 | GGGAGCTGCC | gggaGCTGcc | 11 12 | −301 | 1 00 | 0 88 |
| | NFY_Q6 | TRRCCAATSRN | cctCCAAtggc | 5 181309 | −156 | 1 00 | 0.89 |
| Z score >= | HFH2_01 | NAWTGTTTRTTT | aaaaAACAaaa | 56 365713 | −1211 | 1 00 | 0 94 |
| 1.96 | SRY_02 | NWWAACAAWANW | aaaaACAAaaac | 7 964442 | −1209 | 1 00 | 0 94 |
| | HFH2_01 | NAWTGTTTRTTT | acactaAAGAaaa | 56 365713 | −1205 | 1 00 | 0 87 |
| | SRY_02 | NWWAACAAWANN | aaaaACAAaaca | 3.860390 | −1203 | 1 00 | 0 95 |
| | HFH2_01 | NAWTGTTTRTTT | aacaaAACAaaa | 28 165126 | −1200 | 1 00 | 0 89 |
| | SRY_02 | NWWAAACAAWANN | caaaACAAaaac | 3 800390 | −1198 | 1 00 | 0 94 |
| | HFH2_01 | NAWTGTTTRTTT | accaaaAACAaaa | 56 365713 | −1194 | 1 00 | 0 87 |
| | SRY_02 | NWWMCAAWANN | aaaaACAAaaac | 7 064442 | −1192 | 1 00 | 0.94 |
| | HFH2_01 | NAWTGTTTRTTT | acaaaAAGAata | 28 165126 | −1188 | 1 00 | 0.91 |
| | HFH1_01 | NAWTGTTTATWT | acaaAAACaata | 28 079407 | −1188 | 1 00 | 0 87 |
| | SRY_02 | NWWAACAAWANN | aaaaACAAtaaa | 3 860390 | −1186 | 1 00 | 0 98 |
| | TATA_01 | STATAAAWRNNNNNN | ctaTAAAaacctctg | 3 965815 | −1181 | 1 00 | 0 89 |
| | NF1_Q6 | NNTTGGCNNNNNNCCNNN | gtITGGCcgtgatggagg | 2 199282 | −1140 | 1 00 | 0 93 |
| | CHOP_01 | NNRTGCAATMCCC | aggTGCAagccct | 20 432681 | −1104 | 1 00 | 0 85 |
| | SRY_02 | NWWAACAAWANN | ctgcAAaagt | 3 860390 | 1093 | 1 00 | 0 85 |
| | LYF1_01 | TTTGGGAGR | ttaGGGAga | 7 842719 | 1082 | 1 00 | 0 90 |
| | E2F_02 | TTTSGCGC | gcgaCAAA | 3 546279 | 1071 | 1 00 | 0.91 |
| | GATA1_03 | NNNNNGATAANNGN | tgtgaGATAgtcg | 2 031644 | −1042 | 1 00 | 0 88 |
| | CDPCR3HD_01 | NATYGATSSS | gataGATCgg | 2 349950 | −1037 | 1 00 | 0 97 |
| | NFE2_01 | TGCTGASTCAY | ggCTGAgtctc | 21 950203 | −1009 | 1 00 | 0 87 |
| | CHOP_01 | NNRTGCAATMCCC | atcTGCAaaaccc | 20 432681 | −969 | 1 00 | 0 86 |
| | NF1_Q6 | NNTTGGCNNNNNNCCNNN | aactcacgttGGCAggg | 2.199282 | −938 | 1 00 | 0 85 |
| | SRY_02 | NWWAACAAWANN | tgctTTGTgaaa | 3 860399 | −891 | 1 00 | 0 85 |
| | HFH1_01 | NAWTGTTTATWT | aaatAAACcagt | 28 019407 | −888 | 1 00 | 0 96 |
| | POLY_C | CAATAAANGCNYYYKCTN | aAATAAAccgtttttt | 177 2679419 | 688 | 1 00 | 0 68 |
| | ISRE_01 | CAGTTTCWCTTTYCC | caGTTTttttttcc | 384 173196 | 880 | 1 00 | 0 80 |
| | TALBETAE47_01 | NNNAACAGATGKTNNN | gcagacaICIGagaat | 15 998313 | −859 | 1 00 | 0 85 |
| | GFI1_01 | NNNNNAAATCANNGNNNNNN | catcgagAAICttgrctacaatc | 2 393233 | −854 | 1 00 | 0 68 |
| | SRY_02 | NWWAACAAWANN | gcctACAArca | 3 860390 | −840 | 1 00 | 0 85 |
| | RFX1_01 | NNGTNRCNNRGYAACNN | tacaatccagGCAArta | 7 172878 | −837 | 1 00 | 0 87 |
| | GFI_01 | NNNNNAAATCANNGNNNNNN | caggccaactattGATTctactctt | 2 393233 | −830 | 1 00 | 0 89 |
| | GFI1_01 | NNNNNAAATCANNGNNNNNN | tlgallcIAATCllaggatattgg | 2 393233 | −820 | 1 00 | 0 89 |
| | GATA1_03 | NNNNNGATAANNGN | cttagGATAttggg | 2 031644 | 809 | 1.00 | 0 90 |
| | NFY_Q6 | TRRCCAATSRN | galaTTGGgcl | 5 187369 | 804 | 1 00 | 0 88 |
| | GFI1_01 | NNNNNAAATCANNGNNNNNN | gggctgccaclGATIclgaaatt | 2 393233 | −790 | 1 00 | 0 89 |
| | E47_02 | NNNMRCAGGTGTTMNN | gclgccaCCTGallct | 15 432640 | 796 | 1 00 | 0 88 |
| | LMO2COM_01 | SNNCAGGTGNNN | lgccaCCTGatt | 3 041567 | −794 | 1 00 | 0 93 |
| | MYOD_01 | SRACAGGTGKYG | IgccaCCTGatt | 40 698075 | −794 | 1 00 | 0 92 |
| | SRY_02 | NWWNAACAAWANN | gaaaTTGTctag | 3 888390 | −780 | 1 00 | 0 87 |
| | TH1E47_01 | NNNNGNRTCTGGMWTT | gtacattCTGCtgg | 16 434030 | −694 | 1 00 | 0 85 |
| | CP2_01 | GCNMNAMCMAG | CTCGctggtgg | 3 137246 | −686 | 1 00 | 0 88 |
| | GATA1_03 | NNNNNGATMNNGN | cacagGATAcaaag | 2 031644 | −565 | 1 00 | 0 88 |
| | SRY_02 | NWWAACAAWANN | ggaIACAAagac | 3 860390 | −661 | 1 00 | 0 85 |
| | NRF2_01 | ACCGGAAGNS | accTTCCgac | 6 701850 | −640 | 1 00 | 0.87 |
| | ER_Q6 | NNARGNNANNNTGACCYNN | aaalgglcctcTGACctrc | 10 374054 | 573 | 1 00 | 0 69 |
| | AP1FJ_Q2 | RSTGACTNMNW | tcTGACctcca | 5 142253 | −564 | 1 00 | 0 90 |
| | AP1_Q2 | RSTGACTNMNW | lclGACctcca | 5 142253 | 564 | 1 00 | 0.86 |
| | RORA1_01 | NWAWNNAGGTCAN | cIGACCIccacg | 5 437913 | 563 | 1 00 | 0.93 |
| | GATA1_03 | NNNENNGATAANNGN | ccacaGATAlgcca | 2 031644 | −556 | 1 00 | 0 87 |
| | OCT1_06 | CWNAWTKWSATRYN | agalalgrcATGCa | 8 438364 | −552 | 1 00 | 0 85 |
| | OCT_C | CTNATTTGCATAY | alaagCAAATlaa | 70 265881 | −520 | 1 00 | 0 88 |
| | NKX25_02 | CWFAATTG | aaATTAat | 3 983418 | −514 | 1 00 | 0 86 |
| | TSI1_01 | NNKGAWTWANANTNN | aattAATTaaattta | 4 120840 | −513 | 1 00 | 0 87 |
| | NKX25_02 | CWTAATTG | atTAATta | 3 983418 | −512 | 1 00 | 0 87 |
| | NKX25_02 | CWTAATTG | taATTAaa | 3 983418 | −510 | 1 00 | 0 87 |
| | SRY_02 | NWWAACAAWANN | aaaACAAaggt | 3 860390 | −499 | 1 00 | 0 93 |
| | NF1_06 | NNTTGCCNNNNNCCCNNN | tggTGGCacacgcctta | 2 199282 | −481 | 1 00 | 0 85 |
| | AHRARNT_01 | KNNKNNTYGCGTGCMS | gcaCACGcctllaatc | 14 600483 | −476 | 1 00 | 0 86 |
| | GFI1_01 | NNNNAAATCANNNGNNNNNN | acgcctttAATCccagcactcagg | 2 393233 | −472 | 1 00 | 0 91 |
| | GFI1_01 | NNNNAATCANNGNNNNNNNN | ggtctaaglGATTlccaggcc | 2 393233 | −413 | 1 00 | 0 97 |
| | NKX25_02 | CWTAATTG | aaATTAaa | 3 083418 | −363 | 1 00 | 0 88 |
| | LYF1_01 | TTTGGGAGR | llgcGGAga | 7 842719 | −333 | 1.00 | 0 89 |
| | NF1_Q6 | NNTTGGCNNNNNNCCNNN | IglgggggclGCCAlll | 2 190262 | −305 | 1 00 | 0 88 |
| | NFKB_Q6 | NGGGGAMTTTCCNN | IGGGAgctccal | 30 067003 | −303 | 1 00 | 0 87 |
| | NFKAPPAB_01 | GGGAMTTYCC | GGGAgctgc | 10 361187 | −301 | 1 00 | 0 88 |
| | ER_Q6 | NNARGNNANNNNNGACCTNN | gaactcacaggtGACcgt | 10 374054 | −279 | 1 00 | 0 86 |
| | E47_02 | NNNRCAGGTCTMNN | actcaCAGGgacccg | 15 432640 | 277 | 1 00 | 0 90 |
| | LMO2COM_01 | SNNCAGGTGNNN | tcaCAGGgaca | 3 041587 | −275 | 1 00 | 0 94 |
| | MYOD_01 | SRACAGGTGKYG | tcaCAGGgacc | 40 696075 | −275 | 1 00 | 0 89 |
| | SREBP1_01 | NATCACGTGAY | cacagTGTAcc | 15 355630 | −274 | 1 00 | 0 86 |
| | AP1_Q4 | RSTGACTMANN | ggTGACccgtt | 11 246163 | −270 | 1 00 | 0 86 |
| | AP1_Q2 | RSTGACTNNNW | ggTGACcgtt | 7 895015 | −270 | 1 00 | 0 91 |
| | AP1FJ_Q2 | RSTGACTMMNW | ggTGACccgtt | 7 895015 | −270 | 1 00 | 0 93 |
| | VMYB_01 | AAYAACGGNN | aaccCGTTgtc | 3 427439 | −266 | 1 00 | 0 93 |
| | HF1_Q6 | NNTTGGCNNNNNNCCNNN | gtcccagtgaaGCCAaac | 2 199282 | −242 | 1 00 | 0 90 |
| | PADS_C | NGTGGTCTC | IGIGGIccc | 5 230232 | −169 | 1 00 | 0 89 |
| | GC_01 | NRGGGGCGGGGGCNK | lgglccGCGGtcct | 35 805311 | −167 | 1 00 | 0.87 |
| | SP1_Q6 | NGGGGGCGGGGYN | ggtccGCCCccct | 25 529462 | −166 | 1 00 | 0 88 |
| | NF1_Q6 | NNTTGGCNNNNNNCCNN | caaTGGCaaagtcgcctg | 2 190282 | −152 | 1 00 | 0 85 |
| | E47_02 | NNNMRCAGGTGTMMN | aglagCAGGtgcaata | 15 432640 | 131 | 1 00 | 0 92 |
| | E47_01 | NSNGCAGGTGKNCN | gtaGCAGgtgcaata | 9 748242 | −133 | 1 00 | 0 88 |
| | LMO2COM_01 | SNNCAGGTGNN | tagCAGCgcaa | 3 041567 | −132 | 1 00 | 0 96 |
| | MYOD_01 | SRACAGGTGKYG | tagCAGGgcaa | 40 699075 | −132 | 1 00 | 0 86 |
| | CHOP_01 | NNRTGCAATMCCC | aggTGCAalalcc | 20 432681 | −128 | 1 00 | 0 95 |
| | CAAT_01 | NNNRRCCAATSA | aatcatCCAAag | 3 434507 | −122 | 1 00 | 0 90 |
| | NFY_Q6 | TRRCCAATSRN | tatCCAAtagt | 5 187369 | −120 | 1 00 | 0 92 |
| | GC_01 | NRGGGGCGGGGCNK | agggGGCGgggctg | 35 805311 | −103 | 1 00 | 1 00 |
| | SP1_Q6 | NGGGGGCGGGGYN | agggGGCGgggcl | 25 529462 | −103 | 1 00 | 0 99 |
| | BARBIE_01 | ATNNAAAGCNGRNGG | agcgAAAGtggatgg | 29 452018 | 6 | 1 00 | 0 91 |
| | NKX25_01 | TYAAGTG | gaAAGTg | 3 534570 | 9 | 1 00 | 0 88 |
| | VMYB_01 | AAYAACGGNN | cagAACGgtg | 3 427439 | 34 | 1 00 | 0 90 |
| | GFI1_01 | NNNNNAAATCANNGNNNNN | ggtgagaaAATCcccgaggagggtg | 2 393233 | 40 | 1 00 | 0 90 |
| | NFKB_Q6 | NGGGGAMTTTCCNN | tgagaaaaTCCCcg | 30 067903 | 42 | 1 00 | 0 86 |
| | ZID_01 | NGGTCYATCAYC | gaaggtgGAGCct | 41 225196 | 64 | 1 00 | 0 91 |
| | TH1E47_01 | NNNGNRTCTGGMMTT | ctggagatCTGGggat | 16 434630 | 75 | 1 00 | 0 88 |
| | SREBP1_02 | KATCACCCAC | gtgggGTGAgg | 27 710802 | 94 | 1 00 | 0 94 |
| | NFE2_01 | TGCTGASTCAY | ggCTGAgacac | 21 950203 | 108 | 1 00 | 0 87 |
| | USF_Q6 | GTCACGTGNC | gcCACGttcc | 6 857788 | 114 | 1 00 | 0.87 |
| | IK1_01 | NNNTGGGAATRCC | cagtTCCCtgat | 14 853568 | 116 | 1 00 | 0 88 |
| | GATA1_03 | NNNNNGATAAANNGN | tcctGATAatttg | 2 031644 | 121 | 1 00 | 0 93 |
| | NKX25_02 | CWTAATTG | gaTAATtt | 3 983418 | 125 | 1 00 | 0 86 |
| | E47_02 | NNNRCAGGGTGMNN | ggttcCAGCtgcctac | 15.432640 | 136 | 1 00 | 0 87 |
| | LMO2COM_01 | SNNCAGGTGNN | ttgCAGGtgcct | 3 041567 | 138 | 1 00 | 0 96 |
| | MYOD_01 | SRACAGGTGKYG | ttcCAGGtgccl | 40 699075 | 138 | 1 00 | 0 86 |
| | GATA1_03 | NNNNNGAtAANNGN | ttCCtTATCcttcc | 2 031644 | 164 | 1 00 | 0 95 |
| | NRF2_01 | ACCGGAAGNS | tccTTCCtggg | 6 701850 | 171 | 1 00 | 0 91 |
| | STAF_02 | NNTTCCCAKMATKCMWNCNN | cttcggggagtgTGCGaaaa | 341 255024 | 173 | 1 00 | 0 86 |
| | IK1_01 | NNNTGGGAATRCC | gtgtGGCAaaaat | 14 853568 | 183 | 1 00 | 0 92 |
| | LYF1_01 | TTTGGGAGR | tgtGGActct | 7 842719 | 101 | 1 00 | 0 86 |
| | AP4_Q6 | CWCAGCTGGN | caCAGCggtc | 14 114306 | 210 | 1 00 | 0 90 |
| | AP1_Q2 | RSTGACTNMNW | cagcgGTCAtc | 5 142253 | 212 | 1 00 | 0 88 |
| | AP1FJ_Q2 | RSTGACTNMMNW | cagcgGTCAtc | 5 142253 | 212 | 1 00 | 0 90 |
| | TALBETAE47_01 | NNNAACAGATGKTNNN | gcggtcaTCTGgtac | 48 119467 | 214 | 1 00 | 0 89 |
| | TAL1ALPHAE47_{—} | NNNAACAGATGKTNNN | gccggtcaTCTGgtcac | 48 119407 | 214 | 1 00 | 0 88 |
| | | 01 | | | | | |
| | TAL1BETAITF2_ | NNNAACAGATGKTNNN | gcaggtcaTCTGgtcac | 48 119467 | 214 | 1 00 | 0 88 |
| | | 01 | | | | | |
| | TH1E47_01 | NNNNGNRTCTGGMWTT | gcggtcatCTGGcac | 16 434630 | 214 | 1 00 | 0 86 |
| | AP1F_02 | RSTGACTNMNW | atctgGTCAcc | 7 895015 | 220 | 1 00 | 0 93 |
| | API_Q4 | RSTGACTMANN | atctgGTCAcc | 11 246163 | 220 | 1 00 | 0 86 |
| | API_Q2 | RSTGAGTNMNW | actcgGTCAcc | 7 898015 | 220 | 1 00 | 0 90 |
| | ER_Q6 | NNARGNNANNTGACGYNN | tctgGTCAcctgaggac | 10 374054 | 221 | 1 00 | 0 86 |
| | NF1_Q6 | NNTTGGCNNNNNNCCNNN | gagggacctctGCCAacc | 2 199282 | 233 | 1 00 | 0 95 |
| | NKX25_01 | TYAAGTG | cACTTIc | 3 534570 | 267 | 1 00 | 0 88 |
| | AP1FJ_02 | RSTGACTNMNW | ggcclGTCAcc | 5 142253 | 281 | 1 00 | 0 91 |
| | AP1_Q2 | RSTGACTNMNW | ggcctGTCAcc | 5 142253 | 281 | 1 00 | 0 88 |
| | SREBP1_02 | KATCACCCCAC | tgTCACccccc | 27 710802 | 285 | 1 00 | 0 87 |
| | TH1E47_01 | NNNNGNRTCTGGMWTT | ccccGCAGatctaaa | 16 434630 | 297 | 1 00 | 0 86 |
| | IRF1_01 | SNAAAGYGAAACC | aaTTTCactttat | 81 006772 | 311 | 1 00 | 0 87 |
| | 1RF2_01 | GAAAGYGAAASY | aaTTTCactttat | 59 661305 | 311 | 1 00 | 0 85 |
| | NF1_Q6 | NNTTGGCNNNNNNCCNNN | gagtggaagcccGCAatt | 2 199262 | 341 | 1 00 | 0 93 |
| | NFY_Q6 | TRRCCAATSRN | ccgCCkAAttc | 5 187360 | 350 | 1 00 | 0 89 |
| | OCT1_Q6 | NNNNATGCAAATNAN | ccaATTccatgtag | 11 430842 | 353 | 1 00 | 0 87 |
| | OCT1_08 | CWNAWTKWSATRYN | ccaatllccATGTa | 8 438364 | 353 | 1 00 | 0 92 |
| | OCT1_07 | TNTATGNTAATT | AATTIccatgta | 27 048281 | 355 | 1 00 | 0 88 |
| | CEBP_C | NGWNTKNKGYAAKNNAYA | aaacttgGCAATttccc | 23 615437 | 374 | 1 00 | 0 86 |
| | NF1_Q6 | NNTTGGCNNNNNNCCNNN | ctTGGCaatttccctct | 2 199282 | 377 | 1 00 | 0.95 |
| | NFKAPPABBS_01 | GGGRATTTCC | ggcaaITTCC | 30 669184 | 381 | 1 00 | 0 86 |
| | CREL_01 | SGGRNWTTCC | ggcaatTTCC | 7 203414 | 381 | 1 00 | 0 86 |
| | NFKAPPAB_01 | GGGAMTTYCC | gcaattTCCC | 10 361187 | 382 | 1 00 | 0 85 |
| | IK1_01 | NNNTGGGAATRCC | caatlTCCCctc | 14 883568 | 383 | 1 00 | 0 86 |
| | AP1_Q4 | RSTGACTMANN | tctrtGTCAgc | 11 246163 | 302 | 1 00 | 0 90 |
| | AP1FJ_Q2 | RSTGACTNMNNW | tctctGTCAgc | 7 895015 | 302 | 1 00 | 0 95 |
| | AP1_Q2 | RSTGACTNMNW | tctctGTCAgc | 7 895015 | 392 | 1 00 | 0 95 |
| | ISRE_01 | CAGTTTCWCTTTYCC | caGTTTccctatcgg | 384 173196 | 405 | 1 00 | 0 85 |
| | IK1_01 | NNNTGGGAATRCC | cagtTCCCatc | 14 853568 | 405 | 1 00 | 0.87 |
| | GATA1_03 | NNNNNGATAANNGN | ttccTATCggtat | 2 031641 | 409 | 1 00 | 0 91 |
| | GATA1_03 | NNNNNGATAANNGN | atcggTATCatgaa | 2.031644 | 415 | 1 00 | 0 88 |
| | NF1_Q6 | NNTTGGCNNNNNNCCNNN | tcatgaagcagGCCAag | 2 199282 | 422 | 1 00 | 0 86 |
| | TATA_01 | STATAAAWRNNNNNN | aaaTAAAataacgaa | 3 965615 | 458 | 1 00 | 0.85 |
| | GFI1_01 | NNNNAAATCANNGNNNNNNN | aataacgaAATCaggatggcgtg | 2 393233 | 464 | 1 00 | 0 92 |
| | VMYB_01 | AAYAACGGNN | aatAACCaaa | 3 427439 | 454 | 1 00 | 0 85 |
| | AHRARNT_01 | KNNKNNTYGCGTGCMS | caggaatggCGTGctc | 14 600483 | 475 | 1 00 | 0 93 |
| | AP1_Q4 | RSTGACTMANN | ccTGACtcctc | 11 246163 | 503 | 1 00 | 0 89 |
| | API_Q2 | RSTGACTNMNW | ccTGACtcctc | 7 895015 | 503 | 1 00 | 0 90 |
| | AP1FJ_Q2 | RSTGACTNMNW | ccTGACtcctc | 7 595015 | 503 | 1 00 | 0 93 |
| | BARBIE_Q2 | ATNNAAAGCNGRNGG | taccctcCTTTlgac | 29.452018 | 532 | 1 00 | 0 86 |
| | AP1FJ_Q2 | RSTGACTNMNW | ttTGACtccgg | 7.896015 | 541 | 1 00 | 0 90 |
| | AP1_Q2 | RSTGACTNMNW | ttTGACtccgg | 7 805015 | 541 | 1 00 | 0 88 |
| | AP1_Q4 | RSTGACTMANN | ttTGACIccgg | 11 246163 | 541 | 1 00 | 0 88 |
| | GC_01 | NRGGGGCGGGGCNK | ggagGGCGggccct | 35 805311 | 550 | 1 00 | 0 91 |
| | SP1_Q6 | NGGGGGCGGGGYN | ggagGGCGggccc | 25 529462 | 550 | 1 00 | 0 93 |
| | TH1E47 01 | NNNNGNRTCTGGMaWTT | cttcttctCTGGtttc | 16 434630 | 565 | 1 00 | 0 86 |
| | AHRARNT_01 | KNNKNNTYGCGTGCMS | ccttgggagCGTGact | 14 600483 | 580 | 1 00 | 0 86 |
| | LYF1_01 | TVTGGGAGR | cttGGGAgc | 7 842719 | 581 | 1 00 | 0 86 |
| | AP1FJ_Q2 | RSTGACTNMMNW | cgTGACttgc | 7 895015 | 589 | 1 00 | 0 92 |
| | AP1_Q2 | RSTGACTNMNW | cgTGACtttgc | 7.895015 | 589 | 1 00 | 0.89 |
| | AP1_Q2 | RSTGACTMANW | cgTGACtttgc | 7.895015 | 589 | 1 00 | 0.89 |
| | AP1_Q4 | RSTGACTMANN | cgTGACtttgc | 11 246163 | 589 | 1 00 | 0 89 |
| | IK1_01 | HNNTGGGAATRCC | tcagtTCCCatct | 14 853508 | 613 | 1 00 | 0 88 |
| | E47_01 | NSNGCAGGTGKWCNN | aaggccagCTCCaaa | 9 748242 | 638 | 1 00 | 0 89 |
| | AP4_Q6 | GWCAGCTGGN | gcCAGCtgca | 21 241746 | 641 | 1 00 | 0 91 |
| | AP4_Q5 | NNCAGCTGNN | gcCAGCtgca | 3 060778 | 841 | 1 00 | 0 94 |
| | AP4_Q6 | CWCAGGTGGN | gccaGCTGca | 21 241746 | 641 | 1 00 | 0 94 |
| | AP4_Q5 | NNCAGCTGNN | gccaGCTGca | 3 060778 | 641 | 1 00 | 0 95 |
| | OCT1_Q65 | NNNNATGCAAATNAN | ccagctgrAAATgac | 11 430842 | 642 | 1.00 | 0 88 |
| | AP1_Q2 | RSTGACTNMNW | aaTGACacaga | 7 895015 | 651 | 1 00 | 0 94 |
| | AP1FJ_Q2 | RSTGACTNNWW | aaTGACacaga | 7 805015 | 651 | 1 00 | 0 94 |
| | AP1_Q4 | RSTGACTMANN | aaTGACacaga | 11 246163 | 651 | 1 00 | 0 91 |
| | E47_Q2 | NNNMRCAGGTGTTMNN | ggggcaCCTGgggcg | 15 432840 | 677 | 1 00 | 0 88 |
| | LMO2COM_01 | SNNCAGGTGNNN | ggccaCCTGggg | 3 041567 | 679 | 1 00 | 0 96 |
| | MYOD_01 | SRACAGGTGKYG | ggccaCCTGggg | 40 698075 | 679 | 1 00 | 0 89 |
| | VMYB_01 | AAYAACGGNN | gcgAACGgaa | 3 427439 | 690 | 1 00 | 0 91 |
| | TH1E47_01 | NNNNGNRTCTGGMWTT | accccggICTGGIatg | 16 434630 | 705 | 1 00 | 0 90 |
| | AP1FJ_Q2 | HSTGACTNMNW | gcTGACcgtgg | 5 142253 | 723 | 1 00 | 0 89 |
| | AP1_Q2 | RSTGACTNMNW | gcTGACcgtgg | 5 142253 | 723 | 1 00 | 0 68 |
| | ZID_01 | NGGCTCYATCAYC | gaccgtgGACCcc | 41 225196 | 120 | 1 00 | 0 89 |
| | BARBIE_01 | ATNNAAAGCNGRNGG | acccaagCTTaaac | 29 452018 | 750 | 1 00 | 0 92 |
| | GC_01 | NRGGGGGCGGGGCNK | aagctcCGCCccct | 35 805311 | 267 | 1 00 | 0 97 |
| | SP1_Q6 | NGGGGGCGGGGYN | agctcCGCCccct | 25 520462 | 708 | 1 00 | 0 95 |
| | CREL_01 | SGGRNWTCC | agggtcTTCC | 3 467858 | 703 | 1 00 | 0 92 |
| | TH1E47_01 | NNNGNRTCTGGMWTT | tcttCCAGaccccagc | 16 434630 | 797 | 1 00 | 0 94 |
| | CDP_02 | NWNATCGATTANYNN | gccttcatCGATagc | 21 123980 | 811 | 1 00 | 0 91 |
| | CLDX_01 | NNTATCGATTANYNW | gccttcatCGATagc | 50 240688 | 811 | 1 00 | 0 90 |
| | GATA1_03 | NNNNGATAANGN | tcatcGATAgcccl | 2 031644 | 815 | 1 00 | 0 88 |
| | NF1_Q6 | NNTTGGCNNNNNNNCCNNN | tagcccttccaGCCAatc | 2 199282 | 822 | 1 00 | 0 93 |
| | NRF2_01 | ACCGGAAGNS | accttCCagc | 6.701850 | 825 | 1 00 | 0 85 |
| | GFI1_01 | NNNNAAATCANNGNNNNN | ttccagccAATCagctagaggac | 2 393233 | 828 | 1 00 | 0 88 |
| | HFY_C | NCTGATTGGYIASY | tccagCCAATcagc | 65 593286 | 829 | 1 00 | 0 96 |
| | CAAT_01 | NNNRRCCAATSA | tccagCCAAtca | 3 434507 | 829 | 1 00 | 0 99 |
| | NFY_Q6 | TRRCCAATSRN | cagCCAAtcag | 5 187369 | 831 | 1 00 | 0 96 |
| | AP4_Q6 | CWCAGCTGGN | gacgGCTGg | 14 114396 | 849 | 1 00 | 0 86 |
| | IK1_01 | NNNTGGGAATRCC | cgggttCCattg | 14 853668 | 862 | 1 00 | 0 91 |
| | NFY_Q6 | TRRCCAATSRN | ccaTTGGtca | 5 187369 | 868 | 1 00 | 0 95 |
| | CAAT_01 | NNNRRCCAATSA | ccaTTGGtcact | 3 434507 | 869 | 1 00 | 0 91 |
| | AP1FJ_Q2 | RSTGACTNMNW | cattgGTCAct | 7.895015 | 870 | 1 00 | 0 94 |
| | AP1_Q4 | RSTGACTMANN | cattgGTCAact | 11 246163 | 870 | 1 00 | 0 91 |
| | AP1_Q2 | RSTGACTNMNW | cattgGTCAact | 7 895015 | 870 | 1 00 | 0 91 |
| | OLF1_01 | NCNANTCCCYNGRGARNKGN | gtcactTCCCtagtgattttct | 77 977123 | 875 | 1 00 | 0 86 |
| | IK1_01 | NNNTGGGAATRCC | tcactTCCCagt | 14 853568 | 876 | 1 00 | 0 87 |
| | SRY_02 | NWWWAACAAWANN | tgccTTGttgc | 3 860390 | 905 | 1 00 | 0 89 |
| | GFI1_01 | NNNNNAAATCANNGNNNNNN | ctctttgcgggaGATIattgagg | 2 393233 | 921 | 1 00 | 0 88 |
| | TATA_01 | SIATAAAWRNNNNN | gcgggagaTTAttg | 3 965815 | 927 | 1 00 | 0 85 |
| | AP2_Q6 | MKCCSCNGGGG | gaCCCGcagaca | 12 284970 | 978 | 1 00 | 0 86 |
| | TH1E47_01 | NNNNGNRTCTGGMWTT | acattgttCTGGagcc | 16 434630 | 987 | 1 00 | 0 89 |
| | HF1_Q6 | NNTTGGCNNNNNCCNN | atttgtctggaGCCacac | 2 199282 | 989 | 1 00 | 0 86 |
| | AP4_Q6 | CWCAGCTGGN | caCAGCtcac | 14 114396 | 1004 | 1.00 | 0 88 |
| | AP4_Q6 | CWCAGCTGGN | ctccGCTGtt | 14 114396 | 1040 | 1 00 | 0 85 |
| | Th1E47_01 | NNNGNRTCTGGMWTT | cggtCCAGagtcatca | 16 434630 | 1052 | 1 00 | 0 88 |
| | VMAF_01 | NNNTGCTGACTCAGCANNN | cgggtccagaGTCAcatgg | 168513881 | 1052 | 1.00 | 0 87 |
| | AP1_Q2 | RSTGACTNMNW | ccagaGTCAtc | 7 895015 | 1056 | 1 00 | 0 93 |
| Core | AP1_Q4 | RSTGACTMANW | ccagaGTCAtc | 11 246163 | 1056 | 1.00 | 0 90 |
| similarity >= | AP1FJ_Q2 | RSTGACTNMNW | ccagaGTCAtc | 7 895015 | 1056 | 1 00 | 0 92 |
| 0.99 | SOX5_01 | NNAACAATNN | aaaaCAATaa | 0 681190 | −1185 | 1 00 | 0 99 |
| Template | AP4_Q5 | NNCAGCTGNN | gggcGTGt | 0 508566 | −1122 | 1 00 | 0 85 |
| similarity >= | DELTAEF1_01 | NNNCACCTNAN | gcaAGCTgcaa | 0 538360 | −1107 | 1 00 | 0 96 |
| 0.85 | IK2_01 | NNNYGGGAWNNN | gtatGGGAgag | 0 854442 | −1083 | 1 00 | 0 91 |
| | CMYB_01 | NNNNNNGNCNGTTGNN | aacgacacGTTGatg | 0 594660 | 1065 | 1 00 | 0.92 |
| | GATA1_02 | NNNNNGATANKGNN | IglgGATAgalcg | 0 930257 | −1042 | 1 00 | 0 91 |
| | GATA1_04 | NNCWGATARNNNN | gtgaGAtAgatcg | 0 653180 | −1041 | 1 00 | 0 94 |
| | LMO2COM_02 | NMGATANSG | gaGATAgat | 0 569272 | −1030 | 1 00 | 0 91 |
| | IK2_01 | NNNYGGGAWNNN | agtcTCCTtcac | −0 854442 | −1014 | 1 00 | 0 89 |
| | AP4_Q5 | NNCAGCTGNN | acCAGCttcc | 0 508566 | −994 | 1.00 | 0 86 |
| | IK2_01 | NNNYGGGAWNNN | catcTCCCtta | −0 854442 | −909 | 1 00 | 0 88 |
| | SOX5_01 | NNAACAATNN | cctaCAATcc | 0 681190 | −839 | 1 00 | 0 80 |
| | GATA1_02 | NNNNNGATANKGNN | cttagGATAttggg | 0 930257 | −809 | 1 00 | 0 93 |
| | GATA1_04 | NNCWGATARNNNN | ttagGATAttggg | 0 653180 | −808 | 1 00 | 0 89 |
| | LMO2COM_02 | NMGATANSG | agGATAttg | 0 569272 | −806 | 1 00 | 0 92 |
| | DELTAEF1_01 | NNNCACCTNAN | lgccACCTgat | 0 538380 | −794 | 1 00 | 0 97 |
| | MYOD_Q6 | NNCANCTGNY | gaCACCtgat | 0 781061 | −793 | 1 00 | 0 95 |
| | SOX5_01 | NNAACAATNN | aaATTGlcla | 0 681190 | −779 | 1 00 | 0 86 |
| | SOX5_01 | NNAACAATNN | ggtaCAATtc | 0 681190 | −695 | 1 00 | 0 86 |
| | GATA1_02 | NNNNNGATANKGNN | cacagGATAcaaag | 0 930257 | −665 | 1 00 | 0 89 |
| | GATA1_04 | NNCWGATARNNNN | acagGATACaaag | 0 853180 | −664 | 1 00 | 0 88 |
| | LMO2COM_02 | NMGATANSG | agGATAcaa | 0 569272 | −682 | 1.00 | 0 88 |
| | CEBPB_01 | RNRTKNNGMAAKNN | accttgtGCAAacc | 1 480836 | −851 | 1 00 | 0 94 |
| | DELTAEF1_01 | NNNCAGCTNAN | tccgACCTaaa | 0 538300 | −838 | 1 00 | 0.87 |
| | CEBPB_01 | RNRTKNNGMAAKNN | lcTTGCclgaggl | 1 400836 | −620 | 1 00 | 0 87 |
| | IK2_01 | NNNYGGGAWNNN | gaggTCCCacat | 0 854442 | −611 | 1 00 | 0 95 |
| | DELTAEF1_01 | NNNCACCTTNAN | tctgACCTcca | 0 538360 | −564 | 1 00 | 0 85 |
| | GATA1_02 | NNNNNGATANKGNN | ccacaGATAgcca | 0 930257 | −556 | 1 00 | 0.93 |
| | GATA1_04 | NNCWGATARNNNN | cacaGATAlgcca | 0 653180 | −555 | 1 00 | 0 94 |
| | LMOZCOM_02 | NMGATANSG | caGATAtgc | 0 569272 | −553 | 1 00 | 0 96 |
| | S8_01 | NNNNNYAATTN | acccTAATaagcaat | −1 397267 | −526 | 1 00 | 0 86 |
| | S8_01 | NNNNNYAATTN | ataagcaaTTAatta | −1 397287 | 520 | 1 00 | 0 95 |
| | S8_01 | NNNNNYAATTN | gcaaattATTAaatt | −1 397287 | −516 | 1 00 | 0 97 |
| | S8_01 | NNNNNYAATTN | aactTAATtaaattta | −1 397287 | −514 | 1 00 | 0 99 |
| | IK2_01 | NNNYGGGAWNNN | ttaaTCCCagca | −0 854442 | −466 | 1 00 | 0 95 |
| | AP4_Q5 | NNCAGGTGNN | rcCAGCactc | 0 508568 | −461 | 1 00 | 0 85 |
| | AP4_Q5 | NNCAGCTGNN | caCAGCagtg | 1 794672 | −386 | 1 00 | 0 93 |
| | SB_01 | NNNNNYAATTN | ctctaaaaATTAaaaa | 1 397287 | −369 | 1 00 | 0 93 |
| | MZF1_01 | NGNGGGGA | cttGGGGa | 0 437162 | −334 | 1 00 | 0 96 |
| | IK2_01 | NNNYGGGAWNNN | cttgGGGAgagg | −0 884442 | −334 | 1 00 | 0 89 |
| | IK2_01 | NNNYGGGAWNNN | tgtgGGGAgctg | −0 854442 | −305 | 1 00 | 0 87 |
| | MZF1_01 | NGNGGGGA | IGctGGa | 0 437162 | −305 | 1 00 | 0 99 |
| | AP4_Q5 | NNCAGCTGNN | gggcaGCTcc | 1 794672 | −301 | 1 00 | 0 91 |
| | MYOD_Q6 | NNCANCTGNY | cacaGGTGac | 0 781061 | −274 | 1 00 | 0 91 |
| | DELTAEF1_01 | NNNGACCTNAN | cacAGGIgacc | 0 538380 | −274 | 1 00 | 0 95 |
| | CMYB_01 | NNNNNNGNCNGTTGNN | caggtgaccccGTTGtccc | 0 594660 | 272 | 1 00 | 0 90 |
| | VMYB_02 | NSYAACGGN | ccCGTTgtc | 0 465812 | −265 | 1 00 | 0 95 |
| | IK2_01 | NNNYGGGAWNNN | gttgTCCCcctc | −0 854112 | −262 | 1 00 | 0 91 |
| | MZF1_01 | NGNGGGA | ICCCCctc | 0 437162 | −258 | 1 00 | 0 96 |
| | IK2_01 | NNNYGGGAWNNN | cgtgTCCCagtg | −0 854442 | −245 | 1 00 | 0 93 |
| | AP4_Q5 | NNCAGCTGNN | IgCAGCagga | 0 508566 | 221 | 1 00 | 0 90 |
| | CMYB_01 | NNNNNNGNCNGTTGNN | caggaatcctGTTGtccc | 0 594660 | −216 | 1 00 | 0 89 |
| | IK2_01 | NNNYGGGAWNNN | gttgTCCCtta | 0 854442 | −206 | 1 00 | 0 91 |
| | AP4_Q5 | NNCAGCTGNN | gcggGCTGtg | 0 508666 | −175 | 1 00 | 0 86 |
| | IK2_01 | NNNNYGGGAWNNN | gtggTCCCgcct | −0 854442 | −168 | 1 00 | 0 92 |
| | DELTAEF1_01 | NNNCACCTNAN | agcAGGTgcaa | 0 530360 | −131 | 1 00 | 0 95 |
| | MYOD_Q8 | NNCANCTGNY | agcaGGTGca | 0 147777 | −131 | 1 00 | 0 96 |
| | GATA1_02 | NNNNNGATANKGNN | lgcaaTATCcaata | −0 046677 | −125 | 1 00 | 0 92 |
| | GATA1_04 | NWCWGATARNNNN | lgcaaTATCcaal | 0 653180 | −125 | 1 00 | 0 87 |
| | LMO2COM_02 | NMGATANSG | caaTATCca | 0 569272 | −123 | 1 00 | 0 92 |
| | NKX25_01 | TYAAGTG | cACTTaa | 1 519193 | −33 | 1 00 | 0 98 |
| | IK2_01 | NNNYGGGAWNNN | gcgcTCCCccgc | −0 854442 | −19 | 1 00 | 0 89 |
| | MZF1_01 | NGNGGGGA | ICCCCcgc | 0 437162 | −15 | 1 00 | 0 96 |
| | VMYB_02 | NSYAACGGN | cagAACGgl | 0 465812 | 34 | 1 00 | 0 92 |
| | IK2_01 | NNNYGGCAWNNN | aaaaTCCCgag | −0 854442 | 46 | 1 00 | 0 90 |
| | MZF1_01 | NGNGGGGA | ICCCCgag | 1 679353 | 50 | 1 00 | 0 95 |
| | DELTAEF1_01 | NNNCACCTNAN | ggaAGGTggag | 0 538360 | 83 | 1 00 | 0 95 |
| | IK2_01 | NNNYGGGAWNNN | IctgGGGAtgct | −0 854442 | 82 | 1 00 | 0 89 |
| | MZF1_01 | NGNGGGGA | IcIGGGGa | 0 437162 | 62 | 1 00 | 0 96 |
| | AP4_Q5 | NNCAGCTGNN | gtggGCTGag | 0 508566 | 105 | 1 00 | 0 86 |
| | ARNT_01 | NNNNNCACGTGNNNNN | tgagCACGttccctg | 0 511281 | 111 | 1 00 | 0 88 |
| | IK2_01 | NNNYGGGAWNNN | acglTCCCgat | −0 894442 | 117 | 1 00 | 0 92 |
| | GATA1_02 | NNNNNGATANKGNN | IccclGATAatttg | 0 930257 | 121 | 1 00 | 0 91 |
| | GATA1_04 | NNCWGATARNNNN | ccctGATActtttg | 0 653180 | 122 | 1 00 | 0 95 |
| | SB_01 | NNNNNYAATTN | ctgaTAATllgggglt | −1 397287 | 124 | 1 00 | 0 95 |
| | LMO2COM_02 | NMGATANSG | ctGATAatt | 0 569272 | 124 | 1 00 | 0 91 |
| | GATA_C | NGATAAGNMNN | IGATAAIIIgg | 1 411097 | 125 | 1 00 | 0 90 |
| | MYOD_Q6 | NNCANCTGNY | tccaGGTGcc | −0 141777 | 139 | 1 00 | 0 91 |
| | DELTAEF1_01 | NNNCACCTNAN | tccAGGTgcct | 0 538360 | 139 | 1 00 | 0 95 |
| | IK2_01 | NMNYGGGAWNNN | actcTCCCttgc | −0 854442 | 150 | 1 00 | 0 88 |
| | GATA C | NGATAAGNMNN | cttccTTATCc | 1 411097 | 163 | 1 00 | 0 97 |
| | GATA1_04 | NNCWGATARNNNN | ttccIIAICcttc | 0 653180 | 164 | 1 00 | 0 94 |
| | GATA1_02 | NNNNNGATANKGNN | ttccTATCcttcc | 0 930257 | 164 | 1 00 | 0 95 |
| | IMO2COM_02 | NNGATANSG | ccTATCcl | 0 560272 | 1 66 | 1 00 | 0 96 |
| | CETS1P54_01 | NCMGGAWGYN | tccttCCGgg | 1 244487 | 171 | 1 00 | 0 94 |
| | IK2_01 | NNNYGGGAWNNN | tccgGGGAgtgt | 0 854442 | 175 | 1 00 | 0 86 |
| | MZF1_01 | NGNGGGGA | tccGGGGa | 0 437162 | 175 | 1 00 | 0 95 |
| | IK2_01 | NNNYGGGAWNNN | gtgtGGGAaaca | 0 854442 | 183 | 1 00 | 0 97 |
| | AP4_Q5 | NNCAGCTGNN | caCAGcggtc | 1 794672 | 210 | 1 00 | 0 92 |
| | DELTAEF1_01 | NNNCACCTNAN | ggttACCTcga | 0 538360 | 224 | 1 00 | 0 94 |
| | IK2_01 | NNNYGGGAWNNN | tcgaGGGAcctc | −0 854442 | 231 | 1 00 | 0 90 |
| | CMYB_01 | NNNNNNGNCNGTTGNN | ctgcCAACctacccctcc | 0 694690 | 242 | 1 00 | 0 85 |
| | DELTAEF1_01 | NNNCACCTNAN | ctacACCTcca | 0 538360 | 250 | 1 00 | 0 94 |
| | IK2_01 | NNNYGGGAWNNN | agtgTCCCactt | −0 854442 | 260 | 1 00 | 0 93 |
| | MZF1_01 | NGNGGGGA | cCCCCacc | 0 437162 | 291 | 1 00 | 0 85 |
| | NKX25_01 | TYAAGTG | cACTTa | 1 519193 | 316 | 1 00 | 0 94 |
| | IK2_01 | NNNYGGGAWNNN | aaagTCCCcgag | −0 854442 | 332 | 1 00 | 0 88 |
| | MZF1_01 | NGNGGGGA | ICCCCgag | 1 670353 | 336 | 1 00 | 0 95 |
| | CEBPB_01 | RNRTKNNGMAAKNN | aactttgGCAAtttt | 1 460830 | 375 | 1 00 | 0 96 |
| | IK2_01 | NNNYGGGAWNNN | aattTCCCtctc | −0 854442 | 384 | 1 00 | 0 92 |
| | IK2_01 | NNNYGGGAWNNN | agIITCCCatc | −0 854442 | 406 | 1 00 | 0 94 |
| | GATA1_04 | NNCWGATARNNNN | ttcccTATCggta | 0 653180 | 409 | 1 00 | 0 93 |
| | GATA1_02 | NNNNNGATANKGNN | ttcccTATCggtat | 0 930257 | 409 | 1 00 | 0 97 |
| | LMO2COM_02 | NMGATANSG | ccccTATCgg | 0 560272 | 411 | 1 00 | 0 99 |
| | GATA1_02 | NNNNNGATANKGNN | atcggTATCatgaa | 0 930251 | 415 | 1 00 | 0 92 |
| | GATA1_04 | NNCWGATARNNNN | atcggTTCatga | 0 653180 | 415 | 1 00 | 0 91 |
| | LMO2COM_02 | NMGATANSG | cggTATCat | 0 569272 | 417 | 1.00 | 0 98 |
| | CETS1P54_01 | NCMGGAWGYN | cagTCCGgg | 1 244487 | 445 | 1 00 | 0 92 |
| | MZF1_01 | NGNGGGGA | cggGGGGa | 0 437182 | 451 | 1 00 | 0 98 |
| | IK2_01 | NNNYGGGAWNNN | cgggGGGAaata | −0 854442 | 451 | 1 00 | 0.91 |
| | IK2_01 | NNNYGGGAWNNN | cctgTCCTgac | −0 854442 | 497 | 1 00 | 0 90 |
| | CETS1P54_01 | NCMGGAWGYN | IgacTCCGga | 1 244457 | 543 | 1 00 | 0 87 |
| | CETS1P54_01 | NCMGGAWGYN | tcCGGAgggc | 1 244487 | 547 | 1 00 | 0 90 |
| | IK2_01 | NNNYGGGAWNNN | ccttGGGAgcgt | −0 854442 | 580 | 1 00 | 0 92 |
| | IK2_01 | NNNYGGGAWNNN | cagITCCC9atct | −0 854442 | 614 | 1 00 | 0 94 |
| | CETS1P54_01 | NCMGGAWGYN | agacTCCGgg | 1 244487 | 659 | 1 00 | 0 86 |
| | DELTAEF1_01 | NNNCACCTNAN | ggcACCTggg | 0 538360 | 079 | 1 00 | 0 95 |
| | MYOD_Q6 | NNCANCTGNY | gcCACCtggg | 0 781061 | 680 | 1 00 | 0 91 |
| | VMYB_02 | NSYAACGGN | gcgAACGga | 0 465812 | 690 | 1 00 | 0 93 |
| | GATA1_02 | NNNNNGATANKGNN | tcatcGATAgccct | 0 930257 | 815 | 1 00 | 0 91 |
| | GATA1_04 | NNCWGATARNNNN | catcGATAgcct | 0 653180 | 816 | 1 00 | 0 88 |
| | LMO2COM_02 | NMGATANSG | tcGATAgcc | 0 569272 | 818 | 1 00 | 0 95 |
| | AP4_Q5 | NNCAGCTGNN | atCAGCtacg | 0 508566 | 837 | 1 00 | 0 89 |
| | AP4_Q5 | NNCAGCTGNN | gacgGCTGcg | 1 794677 | 849 | 1 00 | 0 91 |
| | IK2_01 | NNNYGGGAWNNN | gggtTCCCattg | 0 854442 | 863 | 1 00 | 0 97 |
| | IK2_01 | NNNYGGGAWNNN | cactTCCCtagt | 0 854442 | 877 | 1 00 | 0 92 |
| | NKX25_01 | TYAAGTG | cACTTcc | 1 519193 | 877 | 1 00 | 0 88 |
| | IK2_01 | NNNYGGGAWNNN | ttgCGGAgatt | −0 854442 | 925 | 1 00 | 0 89 |
| | AP4_Q5 | NNCAGCTGNN | ctCAGCccga | 0 508566 | 945 | 1 00 | 0 87 |
| | AP4_Q5 | NNCAGCTGNN | caCAGtlcac | 1 794672 | 1004 | 1 00 | 0 92 |
| | AP4_Q5 | NNCAGCTGNN | ctcCGCTGtt | 1 794672 | 1040 | 1 00 | 0 91 |
| | CETS1P54_01 | NCMGGAWGYN | tgttTCCGgt | 1 244481 | 1046 | 1 00 | 0 95 |
| None | HFH2_01 | NAWTGTTTRTTT | aaacaAAAAaaa | 58.365713 | −1219 | 0.62 | 0 89 |
| (MatIspec- | HNF3B_01 | NNNTRTTTRYTY | aaacaAAAAaaa | 6.168471 | −1219 | 0.85 | 0 90 |
| tor) default | HFH3B_01 | NAWTGTTTRTTT | aacaAAAAAcaa | 28.166126 | −1215 | 0 82 | 0 88 |
| (parameters) | HNF3B_01 | NNNTRTTTRYTY | aaaaAACAaaaa | 6.168471 | 1211 | 0 99 | 0 88 |
| | HNF3B_01 | NNNTRTTTRYTY | aaacaAAAAcaa | 9 407093 | −1207 | 0 85 | 0 80 |
| | HNF3B_01 | NNNTRTTTRYTY | aaacaAAAAraa | 9 407093 | −1196 | 0 85 | 0 89 |
| | HNF3B_01 | NNNTRTTTRYTY | aaacaAAAAcaa | 9.407093 | −1190 | 0 85 | 0 89 |
| | HFHZ_01 | NAWTGTTTRTTT | aaaacAATAaaa | 28.165126 | −1185 | 0 90 | 0 85 |
| | TATA_C | NCTATAAAAR | acAATAAAAa | 0 111772 | −1182 | 0 89 | 0 93 |
| | VMYB_01 | AAYAACGGNN | ctcTGTTtct | 3 427439 | −1171 | 0 82 | 0 85 |
| | VMYB_01 | AAYAACGGNN | agaAACAgac | 3 427439 | −1068 | 0 82 | 0.86 |
| | LMO2COM_01 | SNNCAGGTGNNN | acaCAGTtgaat | 1 242813 | −1060 | 0 80 | 0 87 |
| | MYOD_06 | NNCANCTGNY | cacaGTTGaa | −0 147777 | −1059 | 0 87 | 0 89 |
| | OCT1_08 | CWNAWTKWSATRYN | cacagttgaATGAa | 8 438364 | −1059 | 0 83 | 0 86 |
| | VMYB_02 | NSYAACCGN | acAGTTgaa | 0 465812 | −1058 | 0 82 | 0 88 |
| | GATA_C | NGATAAGNMNN | aGATAGatcgg | 1 411097 | −1038 | 0 89 | 0 90 |
| | CEBPB_01 | RNRTKNNNGMAARNN | gggtggaGAAAgag | 1 460836 | −1022 | 0 99 | 0 93 |
| | LMO2COM_01 | SNNCAGGTGNNN | atgcaTCTGcaa | 1.242813 | −973 | 0 82 | 0 91 |
| | MYOD_06 | NNCANCTGNY | tgCATCIgca | −0.147777 | −972 | 0 92 | 0.91 |
| | AP1_C | NTCASTCAN | cTAACTCAc | 1 430304 | −940 | 0 86 | 0 87 |
| | AP1_C | NTGASTCAN | cTAACTCAc | 1 430304 | −940 | 0 85 | 0 87 |
| | PADS_C | NGTGGTGTC | gGTGATcta | 5 230232 | −922 | 0 90 | 0 89 |
| | CEBP_C | NGWNTKNKGYAAKNNAYA | tgctttgIGAAATaaacc | 23 615437 | −897 | 0 80 | 0 89 |
| | CEBPB_01 | RNRTKNNGMAAKNN | gcttgIGAAAIaa | 1 460836 | −896 | 0 99 | 0 95 |
| | VMYB_01 | AAYAACGGNN | accAGTTtttt | 3.427439 | −882 | 0 88 | 0.88 |
| | HFH2_01 | NAWTGTTTRTTT | cagTTTtttt | 28 165126 | −680 | 0 82 | 0 86 |
| | CETS1P54_01 | NCMGGAWGYN | tttTCCAga | 1 244481 | −872 | 0 85 | 0 86 |
| | LMO2COM_01 | SNNCAGGTGNNN | agaacaTCTGaga | 1 242813 | −857 | 0 82 | 0 89 |
| | MYOD_06 | NNCANCTGNY | gaCATCgag | −0 147777 | −856 | 0 92 | 0 89 |
| | SRY_02 | NWWAACAAWANN | actaTTGAttct | 3 860390 | −824 | 0 81 | 0 85 |
| | CDPCR3HD_01 | NATYGATSSS | tattGATTctt | 2 349950 | −822 | 0 89 | 0 93 |
| | OCI1_02 | NNGAATATKCANNN | tcttaGGATattggg | 8 030815 | −810 | 0 86 | 0 86 |
| | GATA_C | NGATAAGNMNN | gGATAttggc | 1 411097 | 805 | 0 87 | 0 86 |
| | USF_Q6 | GYCAGGTGNC | gcGACCtgat | 13 868419 | 793 | 0 82 | 0 89 |
| | USF_C | NCACGTGN | cCACCTga | 0 607662 | −792 | 0 86 | 0 93 |
| | USF_Q6 | GYCACGTGNC | ggctGGTGgr | 6 857788 | 684 | 0 82 | 0 87 |
| | CETS1P54_01 | NGMGGAWGYN | gcAGGAgatg | 1 244487 | −676 | 0 93 | 0 88 |
| | USF_Q6 | GYCAGGTGNC | ggCACAggat | 6 857788 | −667 | 0 86 | 0 85 |
| | CETS1P54_01 | NCMGGAWGYN | aCAGGAtaca | 1 244487 | −664 | 0 93 | 0 91 |
| | GATA_C | NGATAAGNMNN | qGATACaaga | 1 411097 | −661 | 0 88 | 0 89 |
| | CETS1P54_01 | NCMGGAWGYN | ccAGGAaatg | 1 244487 | −578 | 0 93 | 0 92 |
| | GATA_C | NGATAAGNMNN | aGATATgccat | 1 411097 | −552 | 0 87 | 0 94 |
| | OCT1_06 | CWNAWTKWSATRYN | gTATgccatgcat | 0 438364 | −551 | 0 94 | 0 85 |
| | LMO2COM_01 | SNNCAGGTGNNN | algCATGtgtcc | 1 242813 | −539 | 0 62 | 0 89 |
| | USF_Q6 | GYCACGTGNC | tgcaTGTGtc | 6 857788 | 538 | 0 86 | 0 86 |
| | USF_C | NCACGTGN | gcATGTGt | 0 507662 | −537 | 0 88 | 0 93 |
| | USF_C | NCACGTGN | gCATGTgt | 0 507662 | −537 | 0 82 | 0 85 |
| | OCT1_06 | CWNAWTKWSATRYN | attaattaATTTa | 8 438364 | −512 | 0 89 | 0 90 |
| | TATA_C | NCTATAAAAR | aaTTTAAAAa | 8 11772 | −504 | 0 93 | 0 87 |
| | MYCMAX_02 | NANCACGTGNNW | ttactTGTGgtg | 3 484391 | −488 | 0 90 | 0 86 |
| | USF_Q6 | GYCACGTGNC | ggCACAcgcc | 6 857788 | −477 | 0 86 | 0 86 |
| | CETS1P54_01 | NCMGGAWGYN | tcAGGAggca | 1 244487 | −453 | 0 93 | 0 91 |
| | PADS_C | NGTGGTCTC | aGTGATttc | 5 230232 | −404 | 0 90 | 0 91 |
| | CETS1P54_01 | NCMGGAWGYN | gattTCCAg | 1 244487 | −401 | 0 85 | 0 89 |
| | CAAT_01 | NNNRRCCAATSA | gtcagCCACtct | 3 434807 | −371 | 0 83 | 0 85 |
| | NFY_Q6 | TRRCCAATSRN | gagCCACtctc | 5 187369 | −375 | 0 81 | 0 85 |
| | TATA_C | NCTATAAAAR | ITTTTAAAaa | 16 345002 | −350 | 0 93 | 0 88 |
| | TATA_C | NCTATAAAAR | ITTTTAAAaa | 16 345002 | −350 | 0 93 | 0 88 |
| | AP2_Q6 | MKCCCSCNGGCG | gtccttGGGGag | 12 284970 | −337 | 0 98 | 0 85 |
| | CETS1P54_01 | NCMGGAWGYN | acAGGAatgt | 1 244487 | −320 | 0 93 | 0.85 |
| | OCT1_06 | CWNAWTKWSATRYN | gCCATttcaagatg | 8 438364 | 294 | 0 83 | 0 86 |
| | CEBPB_01 | RNRTKNNGMAAKNN | ccaTTTCaagatgt | 1 480838 | 293 | 0 99 | 0 92 |
| | E47_01 | NSNGCAGGTGKNCNN | ctcACAGgtgacccg | 9 748242 | −275 | 0 83 | 0 85 |
| | USF_Q6 | GYCACGTGNC | ctCACAggtg | 6 857788 | −276 | 0 86 | 0 86 |
| | USF_Q6 | GYCACGTGNC | cacaGGTGar | 13 858419 | −274 | 0 82 | 0 89 |
| | USF_C | NCACGTGN | acAGGTGa | 0 507662 | −273 | 0 86 | 0 92 |
| | ARP1_01 | TGACCYTTGANCCYW | tgacccGTTGtccccc | 123 979855 | −268 | 0 83 | 0 87 |
| | CETS1PS4_01 | NCMGGAWGYN | gcAGGAatcc | 1 244487 | −217 | 0 93 | 0 88 |
| | CETS1P54_01 | NCMGGAWGYN | ggaaTCCTgt | 1 244487 | −214 | 0 93 | 0 88 |
| | VMYB_01 | AAYMCGGNN | tccTGTTgtc | 3 427439 | −210 | 0 82 | 0 86 |
| | CEBPB_01 | RNRTKNNGMAAKNN | cctttaaGAAAccc | 1 460836 | −200 | 0 99 | 0 89 |
| | USF_C | NCACGTGN | gcAGCTCc | 0 507662 | −181 | 0 86 | 0 92 |
| | GATA_C | NGATAAGNNNN | gtgcaATATCc | 1 411097 | 126 | 0 87 | 0 89 |
| | OCT1_02 | NNGAATATKCNNNN | tgcctATCCaatag | 8 039815 | −125 | 0 86 | 0 92 |
| | NFKB_C | NGGGACTTTCCA | gagaaaATCCCc | 42 843021 | 43 | 0 93 | 0 88 |
| | NFKAPPAB_01 | GCGAMTTYCC | gaaaaICCC | 10 361187 | 45 | 0 90 | 0 86 |
| | CETS1P54_01 | NCMGGAWGYN | aTGGAgatc | 1 214487 | 74 | 0 85 | 0 85 |
| | RFX1_01 | NNGTNRCNNRGTAACNN | acgTTCCctgataatt | 7 172818 | 117 | 0 88 | 0 85 |
| | CETS1P54_01 | NCNGGAWCYN | gggTCCAgg | 1 244487 | 135 | 0 85 | 0 86 |
| | USF_C | NCACCGTGN | ccAGGTGc | 0 507662 | 140 | 0 86 | 0 92 |
| | CEBPB_01 | RNRTKNGMAAKNN | gagtgtgGGAAaaa | 1 460306 | 181 | 0 87 | 0 88 |
| | LMO2COM_01 | SNNCAGGTGNN | ggtcaTCTGgtc | 1 242813 | 216 | 0 82 | 0 88 |
| | MYOD_Q6 | NNCANCTGNY | gtCATCggt | −0 147777 | 217 | 0 92 | 0 93 |
| | CETS1P54_01 | NCMGGAWGYN | caccTCCAgt | 1 244487 | 253 | 0 85 | 0 91 |
| | USF_Q6 | GTCACGTGNC | ctcaAGTCr | 6 857788 | 256 | 0 86 | 0 86 |
| | CEBPB_01 | RNRTKNNGMAAKNN | cacTTCaaatga | 1 460836 | 267 | 0 99 | 0 93 |
| | SRF_Q6 | GNCCAWATAWGGWN | ttCCAAatgaggrr | 30 107806 | 771 | 0 97 | 0 91 |
| | GC_01 | NRGGGGCGGGGCNK | accccccCACCcccc | 35 805311 | 289 | 0 87 | 0 91 |
| | SP1_Q6 | NGGGGGCGGGYN | cccccCACCcccc | 25 529482 | 290 | 0 92 | 0 92 |
| | OCT1_06 | CWNAWTKWSATRYN | cAAATctcatta | 8 438364 | 309 | 0 89 | 0 85 |
| | CEBPB_01 | RNRTKNNGMAKNN | acttatGAAgaa | 1 460836 | 317 | 0 99 | 0 94 |
| | OCT1_05 | MKNATTTGCATAYY | ccaatttCCATgta | 49 364942 | 363 | 0 85 | 0 86 |
| | CEBPB_01 | RNRTKNNGMAKNN | cagTTTCatcg | 1 460836 | 405 | 0 99 | 0 87 |
| | GATA_C | NGATAAGNNNN | ttttccCTACg | 1 411097 | 408 | 0 89 | 0 93 |
| | GATA_C | NGTAAGMNN | tatgcGTATCa | 1 411097 | 414 | 0 88 | 0 89 |
| | USF_Q6 | GYCACGTGNC | gcCACAggca | 6 857788 | 433 | 0 86 | 0 85 |
| | AP2_Q6 | MKCCCSCNGCCC | ttccggGGGaa | 12 284970 | 448 | 0 98 | 0 85 |
| | OCT1_06 | CWNAWTKWSATRYN | gAAATaaaatacg | 8 438364 | 457 | 0 89 | 0 86 |
| | CEBPB_01 | RNRTKNNGMAAKN | aaataacGAAAtca | 1 460836 | 463 | 0 99 | 0 91 |
| | AP2_Q6 | MKCCCSCNGGG | ctccggAGGGcg | 12 284970 | 546 | 0 86 | 0 91 |
| | RFX1_02 | NNGTNRCNNNNRGYAACNN | tggTTTCcttgggagcyt | 7 174515 | 574 | 0 88 | 0 89 |
| | FRX1_01 | NNGTNRCNNRGYAACNN | tggTTTCcttgggagcg | 7 172878 | 574 | 0 88 | 0 88 |
| | LMO2COM_01 | SNNCAGGTGNN | ggcCAGCgaaa | 1 242813 | 640 | 0 88 | 0 94 |
| | LMO2COM_01 | SNNCAGGTGNN | ggcCAGCgaaa | 1 242813 | 640 | 0 88 | 0 91 |
| | MYOD_Q6 | NNCANCTGNY | gcraGCTGca | 1 709698 | 641 | 0 92 | 0 97 |
| | MYOD_Q6 | NNCANCTGNY | gcraGCTGca | 1 709698 | 641 | 0 92 | 0 90 |
| | AP1_C | NTGASTCAN | atGACACAg | 1 430304 | 652 | 0 86 | 0 85 |
| | USF_Q6 | GYCACGTGNC | ctCACCgggg | 6 857788 | 670 | 0 82 | 0 85 |
| | USF_Q6 | GYCACGTGNC | ctCACCgggg | 13 858419 | 680 | 0 82 | 0 89 |
| | USF_C | NCACGTGN | cCACCTgg | 0 507662 | 681 | 0 86 | 0 92 |
| | RFX1_02 | NNGTNRCNNNRGYAACNN | ctggggcgaacGGAAccg | 7 174515 | 685 | 0 88 | 0 89 |
| | AP2_Q6 | MKCCCSCNGGCG | agCCCCgagccc | 12 284970 | 734 | 0 98 | 0 85 |
| | OCT1_Q6 | NNNNATGCMATNAN | aagaatgcAAACagg | 11.430842 | 781 | 0 80 | 0.88 |
| | HNF3B_01 | NNNTRTTTRYTY | atgcaAACAggg | 2 929849 | 785 | 0 99 | 0 92 |
| | CETS1P54_01 | NCMGGAWGYN | gtctTCCAga | 1 244487 | 796 | 0 85 | 0.89 |
| | CDPCR3HD_01 | NATYGATSSS | ttCATCgata | 2 349950 | 814 | 0 93 | 0 93 |
| | CDPCR3HD_01 | NATYGATSSS | catcGATAgc | 2 340950 | 816 | 0 84 | 0.95 |
| | GATA_ | NGATMGNMNN | cGATAGCccctt | 1 411097 | 819 | 0 89 | 0 88 |
| | CETS1P54_01 | NCMGGAWGYN | ccctTCCAgc | 1 244487 | 825 | 0 85 | 0 89 |
| | HNF3B_01 | NNNTRTTTRYTY | cctTGTTgccg | 2.929849 | 907 | 0 99 | 0.88 |
| | CETS1P54_01 | NCMGGAWGYN | tttgTCCTgt | 1 244487 | 1027 | 0 93 | 0 86 |

TABLE 3

| Promoter | Site | Transcripton Factor | Core Similarity | Matrix Similarity | Z_score | Position/TSS (bp) | motif | consensus |
|---|---|---|---|---|---|---|---|---|
| ABCA7 Human | A | GFI-01 | 1,00 | 0,88 | 4,43 | −569 | GCCACTATAATCGGAGACTCTAGA | NNNNNNAAATCANNGNNNNNNNN |
| | B | HNF3B_03 | 0,99 | 0,85 | 4,37 | −547 | GAATGTTGGCCC | NNNTRTTTRYTY |
| | C | CEBP_01 | 0,87 | 0,85 | 2,13 | −498 | CGTTCGTGGAATGA | RNRTKNNGMAAKNN |
| | D | CEBP_01 | 0,87 | 0,85 | 2,13 | −469 | ATCTAGTGGAACCC | RNRTKNNGMAAKNN |
| | E | NF1_Q6 | 1,00 | 0,86 | 2,00 | −402 | GCCTGGCCAGCCCCGGGG | NNTTGGCNNNNNNCCNNN |
| | F | AP4_Q5 | 1,00 | 0,90 | 1,68 | −340 | TGCAGCCGGT | NNCAGCTGNN |
| | G | NFKAPPAB_01 | 1,00 | 0,90 | 9,96 | −260 | GGGACCTGCC | GGGAMTTYCC |
| | H | NF1_Q6 | 1,00 | 0,89 | 2,00 | −106 | CGCCCAATAGC | TRRCCAATSRN |
| ABCA7 Mouse | A | GFI-01 | 1,00 | 0,88 | 2,96 | −842 | TTGCCTACAATCCAGGCAACTATT | NNNNNNAAATCANNGNNNNNNNN |
| | B | HNF3B_03 | 0,99 | 0,85 | 3,25 | −825 | AACTATTGATTC | NNNTRTTTRYTY |
| | C | CEBP_01 | 0,99 | 0,94 | 1,72 | −787 | TGATTCTGAAATTG | RNRTKNNGMAAKNN |
| | D | CEBP_01 | 1,00 | 0,91 | 1,72 | −760 | ATGTTGCTAAAATG | RNRTKNNGMAAKNN |
| | E | NF1_Q6 | 1,00 | 0,88 | 2,61 | −688 | TTCTGGCTGGTGGTGGCAGGA | NNTTGGCNNNNNNCCNNN |
| | F | AP4_Q5 | 1,00 | 0,93 | 2,03 | −386 | CACAGCAGTG | NNCAGCTGNN |
| | G | NFKAPPAB_01 | 1,00 | 0,88 | 11, 12 | −301 | GGGAGCTGCC | GGGAMTTYCC |
| | H | NFY_Q6 | 1,00 | 0,89 | 5,63 | −156 | CCTCCAATGGC | TRRCCAATSRN |

TABLE 4
Oligonucleotides Specific
for the Human ABCA7 Gene

| Name | Sequence (5′-3′) | Orientation |
|---|---|---|
| ABCA7_U2 | CTTCAGCCCGACCGTTG | Sense |
| ABCA7_AJ | AGAATTTCATGTATCGCC | Sense |
| ABCA7_L2 | CGATGGCAGTGGCTTGTTTGG | Antisense |
| ABCA7_L1 | GCGGAAAGCAGGTGTTGTTCAC | Antisense |
| ABCA7_AL | CTGGAGTTGCTGTCAGAG | Sense |
| ABCA7_AK | GGGTAAAAGGTGTATCTGG | Antisense |
| ABCA7_AN | TCACGAGGACCAATAAGATC | Sense |
| ABCA7_AM | TGTCAGTGTCACGGAGTAG | Antisense |
| ABCA7_AP | CCTGGAAGCTGTGTGC | Sense |
| ABCA7_AO | ACGGAGACGCCAGGAC | Antisense |
| ABCA7_AR | GTCCTGGCGTCTCCGTTC | Sense |
| ABCA7_AQ | CTCGTCCAGGATAACAAC | Antisense |
| ABCA7_AT | GTGCTGCCCTACACGG | Sense |
| ABCA7_AS | CAGTGCCCAGCCCTGTAC | Antisense |
| ABCA7_AV | ACCCCAGAGTCTCCATCC | Sense |
| ABCA7_AU | GAGAAGCCTCCGTATCTGAC | Antisense |
| ABCA7_AX | CTGCTCTCCTGCTGTTGC | Sense |
| ABCA7_AW | GCACCATGTCAATGAGCC | Antisense |
| ABCA7_AZ | CCTCAGCATGGGATACTG | Sense |
| ABCA7_AY | GCTTGCGTTTGTTCCCTC | Antisense |
| ABCA7_BA | ACCACGGCTTCTCTCC | Antisense |
| ABCA7_Q | AGCCAGCAACGCAATCCTCC | Sense |
| ABCA7_B | CGCACCATGTCAATGAGCCC | Antisense |
| ABCA7_L3 | TGAAGACGTGCGGTGCG | Antisense |
| ABCA7_L4 | TGTCTCCGGCGATACATGAAATTC | Antisense |
| ABCA7_L5 | ACCTCAGACCCAGACCCTTACGC | Antisense |
| ABCA7_U4 | GGAATGAGGTTCAGAAAGGG | Sense |
| ABCA7_U5 | ATGCAAGTTCCCTGGGAGTTAG | Sense |
| ABCA7_U6 | CTCCTTCCGGTGAATGTTGACG | Sense |

We claim:

1. Nucleic acid comprising a polynucleotide having at least 20 consecutive nucleotides having the nucleotide sequence chosen from the sequences SEQ ID No. 1-5, or a nucleic acid having a complementary sequence.

2. Nucleic acid having at least 80% nucleotide identity with a nucleic acid according to claim 1.

3. Nucleic acid hybridizing, under high stringency hybridization conditions, with a nucleic acid according to claim 1 or 2.

4. Nucleic acid according to one of claims 1 to 3, capable of modulating the transcription of a polynucleotide placed under its control.

5. Nucleic acid according to claim 4, comprising a polynucleotide ranging from the nucleotide at position −1 to the nucleotide at position −1111 relative to the first nucleotide transcribed, located at position 1112 of the nucleotide sequence SEQ ID No. 1.

6. Nucleic acid according to claim 4, capable of activating the transcription of a polynucleotide of interest placed under its control.

7. Nucleic acid according to claim 4, capable of inhibiting the transcription of a polynucleotide of interest placed under its control.

8. Nucleic acid comprising:
a) a nucleic acid according to one of claims 1 to 7; and
b) a polynucleotide encoding a polypeptide or a nucleic acid of interest.

9. Nucleic acid according to claim 8, characterized in that the nucleic acid of interest is an oligonucleotide of the sense or antisense type.

10. Recombinant cloning and/or expression vector comprising a nucleic acid according to one of claims 1 to 9.

11. Host cell transformed with a nucleic acid according to one of claims 1 to 9 or with a recombinant vector according to claim 10.

12. Nonhuman transgenic mammal whose somatic cells and/or germ cells have been transformed with a nucleic acid according to one of claims 1 to 9 or with a recombinant vector according to claim 10.

13. Method for screening a substance or a molecule modulating the transcription of the constitutive polynucleotide of the nucleic acid according to claim 8, characterized in that it comprises the following steps:
a) culturing a host cell transformed according to claim 11;
b) incubating the transformed host cell in the presence of the candidate substance or molecule;
c) detecting the expression of the polynucleotide of interest;
d) comparing the results of the detection obtained in step c) with the results of the detection obtained by culturing the transformed host cell in the absence of the candidate molecule or substance.

14. Kit or box for the in vitro screening of a candidate molecule or substance modulating the transcription of the polypeptide of interest encoded by a constitutive polynucleotide of the nucleic acid according to claim 8, comprising:
a) a host cell transformed according to claim 11;
b) where appropriate, the means necessary for the detection of the transcription of the constitutive polynucleotide of interest of the nucleic acid according to claim 8.

15. Method of in vivo screening of a substance or molecule modulating the transcription of a constitutive polynucleotide of interest of the nucleic acid according to claim 8, characterized in that it comprises the following steps:
a) administering the candidate substance or molecule to a nonhuman transgenic mammal according to claim 12;
b) detecting the expression of the polynucleotide of interest in the transgenic mammal as treated in step a);
c) comparing the results of detection of step b) to the results observed with a nonhuman transgenic mammal according to claim 12 which has not received the administration of the candidate substance or molecule.

16. Kit or box for the in vivo screening of a candidate molecule or substance modulating the transcription of the constitutive polynucleotide of interest of the nucleic acid according to claim 8, comprising:
a) a nonhuman transgenic mammal according to claim 12;
b) where appropriate, the means necessary for the detection of the transcription of said polynucleotide of interest.

17. Substance or molecule modulating the transcription of a constitutive polynucleotide of interest of the nucleic acid according to claim 8.

18. Substance or molecule according to claim 17, characterized in that it is selected according to the method of claim 13 or of claim 15.

19. Pharmaceutical composition comprising, as active ingredient, a substance or a molecule according to either of claims 17 and 18.

20. Pharmaceutical composition according to claim 19, characterized in that it is intended for the treatment and/or prevention of deficiencies in the metabolism of lipids, or in the mechanisms involving the immune system and inflammation.

21. Substance or molecule according to either of claims 17 and 18, as active ingredient for a medicament.

22. Method of detecting an impairment of the transcription of the ABCA7 gene in a subject, comprising the following steps:
a) extracting the total messenger RNA from a biological material obtained from the subject to be tested;
b) quantifying the messenger RNA for ABCA7 present in said biological material;
c) comparing the quantity of messenger RNA for ABCA7 obtained in step b) with the quantity of messenger RNA for ABCA7 expected in a normal subject.

23. Method of detecting an impairment of the transcription of the ABCA7 gene in a subject, comprising the following steps:
a) sequencing, from a biological material obtained from the subject to be tested, a polynucleotide located upstream of the site of initiation of transcription of the ABCA7 gene;
b) aligning the nucleotide sequence obtained in a) with the sequence SEQ ID No. 1;
c) determining the various nucleotides between the sequenced polynucleotide obtained from the biological material of the subject to be tested and the reference sequence SEQ ID No. 1.

24. Kit or box for the detection of an impairment of the transcription of the ABCA7 gene in a subject, comprising the means necessary for quantifying the messenger RNA for ABCA7 in a biological material obtained from said subject to be tested.

25. Kit or box for the detection of an impairment of the transcription of the ABCA7 gene in a subject, comprising the means necessary for the sequencing of a polynucleotide located upstream of the site of initiation of transcription of the ABCA7 gene in the subject to be tested.

26. Method of screening a molecule or substance modulating the transcription of the constitutive polynucleotide of interest of the nucleic acid according to claim 8, comprising the following steps:
a) incubating a nucleic acid according to one of claims 1 to 9 or a recombinant vector according to claim 10 with a candidate molecule or substance to be tested;
b) detecting the complex formed between the candidate molecule or substance and the candidate molecule or substance.

27. Kit or box for the screening of a candidate molecule or substance modulating the transcription of the constitutive polynucleotide of interest of the nucleic acid according to claim 8 comprising:
a) a nucleic acid according to one of claims 1 to 9 or a recombinant vector according to claim 10;
b) where appropriate, the means necessary for the detection of the complex formed between the candidate molecule or substance and said nucleic acid.


SEQUENCE LISTING (12 sequences)

SEQ ID NO: 1 - DNA, 2322 bp, Homo sapiens
aaaacctctgtttgtacgaagagaaggtggccaagagagttggcgtcgatgagggcgtgctttgctttgatgcttttgtggggagagaggaggtcttgggggatggggggatcaaggggaaaatgtccacctcaccattgggaggaggagcaaaagctgaagccacaggtgagtctgggtggaatgaatgatttgaagggccgggacttggggtagagggagaggctgggcttcctggccatttggagaagaggcagttccctcaaatgccccccatgcgctttggctgcactctaccttacagcgcaagtctcgtggcctcagcctggatgtctccccgttggcgaactcctatttatcctcaaagccccaacggcaatgccacctcctgccgcgggagccgtccccacgcctctcactctccccagcgccttcaaagctgtggacccacacgctcccatttcagcttcacctccagcctgaagagtttatttcaactcttcttccagagtgggaaacgggttttcctcaaaatcagggtagccactataatcggagactctagaatgttggccccctccccctcctgccatcctctgcagaagccgaggagcgttcgtggaatgaatgaatgaacgaatgatctagtggaacccctactttacagacggacgagtgtagtcccagagtctggactaaactagagggagcctggccagccccggggacagcggggacagagggaactcctgcaattcggagctgcggtattgcagccggttatacaacgtggggaggcagcctggctccccaaagacagcgcagcctcgttcccggagggcggcctgcctgggacctgccgggcactccgccaccctacggtgatgcagcaagagccgcgcggtccctttaagaaacccggctaggcgaggcccttctgtgatcccgtctcctcccttggcccgcgcagctccgacggagcaggccagtgagtgacgggcaggtcgcccaatagcagcgtgcagaggcaggggcgtgccccggcgctgctacctgcgcgggcaagctcagcgcacttggcttaaggggcggcgcgctccctgcctgctgctgggcggagggaaggcggcaagagctgcggagcccctggaaggtgagaaggactcggagagggaagaaggcccgagactcgagaatgcggggttggggccgggagggatgcaagttccctgggaattagggggtccagcctctgacctccttccggtgaatgttgacgacggctgaattgatcactgattctcaaggggggcatcggacatctgggacccttaagagggcctttgccgatcacacacctgcagccccctgcccgttagaactcctgcactcccccttgccccgtcttacaaatggagaaactgagcccactcccccagatcctaagtcccgcttgatgtaaaggaaagaaccctggcgtaagggtctgggtctgaggtcccagttccggcctggtcacctttagcaacttcctgcccctctgtcagcgtcagattctccatctgtgtcagaggtggaccggcccaaggaaaatagatcaggaatcgctgactccaggagtctctatcccagccccttcgcctgactctttctctggctcccgcggtccctctgagcgattaatgctacataaggtgtgggcagagctggggtcgtgcctccagctgggcaactgcctgtctctctgggtgcctgggtttgctttcttgggcctcggtttccacttctgtagagtggggtgatagtccagcacttcccctgggcgtgtgaaatgtccagcactgccaatattcgttgctgttatcttcggagaacagtgaggggaaaggaatccttgcctgggctgggccaggcaggaggctgggggtcaggacctggaagaggcttccaggtgaggcttggggtggagcctggtgacgaaagcgttaagcccaaactcggtccctggaggattagaggatgatctttaagtccccagctgtcagccctgctcagagcgacagtcctggcagccaatcagatgcgaggacggctgcgggttgcgctcccattggtttactccacccctggggtagcggagcctctttatcgagtgactactgtttgcctcgctctaatcagagcttccaggaaccctgcgctgtgggataaaggaatgaggttcagaaaggggcagggagttgcccgcagccgcaccgcacgtcttcagcccgaccgttgtcctgacctctctgtcccgtcccctgcccagtctcaccatgg

SEQ ID NO: 2 - DNA, 1111 bp, Homo sapiens
aaaacctctgtttgtacgaagagaaggtggccaagagagttggcgtcgatgagggcgtgctttgctttgatgcttttgtggggagagaggaggtcttgggggatggggggatcaaggggaaaatgtccacctcaccattgggaggaggagcaaaagctgaagccacaggtgagtctgggtggaatgaatgatttgaagggccgggacttggggtagagggagaggctgggcttcctggccatttggagaagaggcagttccctcaaatgccccccatgcgctttggctgcactctaccttacagcgcaagtctcgtggcctcagcctggatgtctccccgttggcgaactcctatttatcctcaaagccccaacggcaatgccacctcctgccgcgggagccgtccccacgcctctcactctccccagcgccttcaaagctgtggacccacacgctcccatttcagcttcacctccagcctgaagagtttatttcaactcttcttccagagtgggaaacgggttttcctcaaaatcagggtagccactataatcggagactctagaatgttggccccctccccctcctgccatcctctgcagaagccgaggagcgttcgtggaatgaatgaatgaacgaatgatctagtggaacccctactttacagacggacgagtgtagtcccagagtctggactaaactagagggagcctggccagccccggggacagcggggacagagggaactcctgcaattcggagctgcggtattgcagccggttatacaacgtggggaggcagcctggctccccaaagacagcgcagcctcgttcccggagggcggcctgcctgggacctgccgggcactccgccaccctacggtgatgcagcaagagccgcgcggtccctttaagaaacccggctaggcgaggcccttctgtgatcccgtctcctcccttggcccgcgcagctccgacggagcaggccagtgagtgacgggcaggtcgcccaatagcagcgtgcagaggcaggggcgtgccccggcgctgctacctgcgcgggcaagctcagcgcacttggcttaaggggcggcgcgctccctgcctgctgc

SEQ ID NO: 3 - DNA, 1211 bp, Homo sapiens
tgggcggagggaaggcggcaagagctgcggagcccctggaaggtgagaaggactcggagagggaagaaggcccgagactcgagaatgcggggttggggccgggagggatgcaagttccctgggaattagggggtccagcctctgacctccttccggtgaatgttgacgacggctgaattgatcactgattctcaaggggggcatcggacatctgggacccttaagagggcctttgccgatcacacacctgcagccccctgcccgttagaactcctgcactcccccttgccccgtcttacaaatggagaaactgagcccactcccccagatcctaagtcccgcttgatgtaaaggaaagaaccctggcgtaagggtctgggtctgaggtcccagttccggcctggtcacctttagcaacttcctgcccctctgtcagcgtcagattctccatctgtgtcagaggtggaccggcccaaggaaaatagatcaggaatcgctgactccaggagtctctatcccagccccttcgcctgactctttctctggctcccgcggtccctctgagcgattaatgctacataaggtgtgggcagagctggggtcgtgcctccagctgggcaactgcctgtctctctgggtgcctgggtttgctttcttgggcctcggtttccacttctgtagagtggggtgatagtccagcacttcccctgggcgtgtgaaatgtccagcactgccaatattcgttgctgttatcttcggagaacagtgaggggaaaggaatccttgcctgggctgggccaggcaggaggctgggggtcaggacctggaagaggcttccaggtgaggcttggggtggagcctggtgacgaaagcgttaagcccaaactcggtccctggaggattagaggatgatctttaagtccccagctgtcagccctgctcagagcgacagtcctggcagccaatcagatgcgaggacggctgcgggttgcgctcccattggtttactccacccctggggtagcggagcctctttatcgagtgactactgtttgcctcgctctaatcagagcttccaggaaccctgcgctgtgggataaaggaatgaggttcagaaaggggcagggagttgcccgcagccgcaccgcacgtcttcagcccgaccgttgtcctgacctctctgtcccgtcccctgcccagtctcaccatgg

SEQ ID NO: 4 - DNA, 2291 bp, Mus musculus
aaaacaaaaaaaaaaacaaaaacaaaacaaaaacaaaaacaataaaaacctctgtttctaagagtaaagtacattcctgagtttggccgtgatggagggggcgctgtctagaagcaaggtgcaagccctgcacaaaagttagggagaaggcgagaaacagacacagttgaatgaatgatgtgagatagatcggggctagggtggagaaagaggctgagtctccctcaccagcttccttcgaactcctatgcatctgcaaaaccccaacttctaaggccccctaactcacgcttgccagggtgatctacacccatctccctctatgctttgtgaaataaaccagttttttttttccagagtaggagacatctgagaatcttgcctacaatccaggcaactattgattctaatcttaggatattgggctgccacctgattctgaaattgtctagaccagaggatgttgctaaaatgaatgtgcaggtccttgaagctctactttggagatgagctcacagaggctgtggtacaattctggctggtggcaggagatggcacaggatacaaagaccttgtgcaaaccttccgacctaaacttggtctttgcctgaggtcccacatcatggtaggcaagaatagactccaggaaatggtcctctgacctccacagatatgccatgcatgcatgtgtcctaccctaataagcaaattaattaaatttaaaaacaaaggttacttgtggtggcacacgcctttaatcccagcactcaggaggcagaggcaggcggatctctgtgagaccagcctggtctaagcagtgatttccaggcctaccacagcagtgtgagccactctcaaaattaaaaagtatttttaaaaaggagtccttggggagaggagacaggaatgtcttgctgtggggagctgccatttcaagatgtgaactcacaggtgacccgttgtccccctctttgtcgtgtcccagtgaagccaaactgatgcagcaggaatcctgttgtccctttaagaaacccggctcggagaggcgggctgtggtcccgcctcctccaatggcaaagtcgcctgagtagcaggtgcaatatccaatagtagcgttagggggcggggctgggtgctccttagggcaccgggttgcgaagggcgtcgtccgcaattgagcggggctccacttaaaggggccgcgctcccccgccgaggccgagaggagcgaaagtggatggagtttgggggcctcagaacggtgagaaaatccccgagagggtggaaggtggagcctggagatctggggatgctgtggggtgagggtgggctgagccacgttccctgataatttggggttccaggtgcctactctcccttgcccttccttatccttccggggagtgtgggaaaaatggaccaccgatcctcacagcggtcatctggtcacctcgagggacctctgccaacctacacctccagtgtcccactttccaaatgaggcctgtcaccccccaccccccagatctcaaatttcactttatgaaagaaaaaagtccccgagtggaagccgccaatttccatgtagatggttaaactttggcaatttccctctctgtcagcctcagtttccctatcggtatcatgaagcaggccacaggcatacagttccggggggaaataaaataacgaaatcaggaatggcgtgctcaaggagcctgtccctgactcctcctagccggcggtcttctgtaccctccttttgactccggagggcgggccctccttcttctctggtttccttgggagcgtgactttgcccctttttgagcctcagttcccatctcttaaaaaatagaaggccagctgcaaatgacacagactccgggtctcaccgggggccacctggggcgaacggaaccgagaccccggtctggtatgaggctgaccgtggagccccgagccccaagccccaagctttaaacccaagctccgccccctaagaatgcaaacagggtcttccagaccccagccttcatcgatagcccttccagccaatcagctacgaggacggctgcgcgccgggttcccattggtcacttccctagtgaatttctttctatggtgccttgtttgccgggctctttgcgggagatttattgaggctcagcccgatgttcggaaggatgaggatcagagacccgcagacatttgtctggagccacacagctcactctcagccttttctttgtcctgtcctctccgctgtttccggtccagagtcatcatgg

SEQ ID NO: 5 - DNA, 1220 bp, Mus musculus
aaaacaaaaaaaaaaacaaaaacaaaacaaaaacaaaaacaataaaaacctctgtttctaagagtaaagtacattcctgagtttggccgtgatggagggggcgctgtctagaagcaaggtgcaagccctgcacaaaagttagggagaaggcgagaaacagacacagttgaatgaatgatgtgagatagatcggggctagggtggagaaagaggctgagtctccctcaccagcttccttcgaactcctatgcatctgcaaaaccccaacttctaaggccccctaactcacgcttgccagggtgatctacacccatctccctctatgctttgtgaaataaaccagttttttttttccagagtaggagacatctgagaatcttgcctacaatccaggcaactattgattctaatcttaggatattgggctgccacctgattctgaaattgtctagaccagaggatgttgctaaaatgaatgtgcaggtccttgaagctctactttggagatgagctcacagaggctgtggtacaattctggctggtggcaggagatggcacaggatacaaagaccttgtgcaaaccttccgacctaaacttggtctttgcctgaggtcccacatcatggtaggcaagaatagactccaggaaatggtcctctgacctccacagatatgccatgcatgcatgtgtcctaccctaataagcaaattaattaaatttaaaaacaaaggttacttgtggtggcacacgcctttaatcccagcactcaggaggcagaggcaggcggatctctgtgagaccagcctggtctaagcagtgatttccaggcctaccacagcagtgtgagccactctcaaaattaaaaagtatttttaaaaaggagtccttggggagaggagacaggaatgtcttgctgtggggagctgccatttcaagatgtgaactcacaggtgacccgttgtccccctctttgtcgtgtcccagtgaagccaaactgatgcagcaggaatcctgttgtccctttaagaaacccggctcggagaggcgggctgtggtcccgcctcctccaatggcaaagtcgcctgagtagcaggtgcaatatccaatagtagcgttagggggcggggctgggtgctccttagggcaccgggttgcgaagggcgtcgtccgcaattgagcggggctccacttaaaggggccgcgctcccccgccgaggcc

SEQ ID NO: 6 - DNA, 1273 bp, Homo sapiens
tgggcggagggaaggcggcaagagctgcggagcccctggaaggtgagaaggactcggagagggaagaaggcccgagactcgagaatgcggggttggggccgggagggatgcaagttccctgggaattagggggtccagcctctgacctccttccggtgaatgttgacgacggctgaattgatcactgattctcaaggggggcatcggacatctgggacccttaagagggcctttgccgatcacacacctgcagccccctgcccgttagaactcctgcactcccccttgccccgtcttacaaatggagaaactgagcccactcccccagatcctaagtcccgcttgatgtaaaggaaagaaccctggcgtaagggtctgggtctgaggtcccagttccggcctggtcacctttagcaacttcctgcccctctgtcagcgtcagattctccatctgtgtcagaggtggaccggcccaaggaaaatagatcaggaatcgctgactccaggagtctctatcccagccccttcgcctgactctttctctggctcccgcggtccctctgagcgattaatgctacataaggtgtgggcagagctggggtcgtgcctccagctgggcaactgcctgtctctctgggtgcctgggtttgctttcttgggcctcggtttccacttctgtagagtggggtgatagtccagcacttcccctgggcgtgtgaaatgtccagcactgccaatattcgttgctgttatcttcggagaacagtgaggggaaaggaatccttgcctgggctgggccaggcaggaggctgggggtcaggacctggaagaggcttccaggtgaggcttggggtggagcctggtgacgaaagcgttaagcccaaactcggtccctggaggattagaggatgatctttaagtccccagctgtcagccctgctcagagcgacagtcctggcagccaatcagatgcgaggacggctgcgggttgcgctcccattggtttactccacccctggggtagcggagcctctttatcgagtgactactgtttgcctcgctctaatcagagcttccaggaaccctgcgctgtgggataaaggaatgaggttcagaaaggggcagggagttgcccgcagccgcaccgcacgtcttcagcccgaccgttgtcctgacctctctgtcccgtcccctgcccagtctcaccatggccttctggacacagctgatgctgctgctctggaagaatttcatgtatcgccggagacagccg

SEQ ID NO: 7 - protein, 22 aa, Homo sapiens
MAFWTQLMLLLWKNFMYRRRQP

SEQ ID NO: 8 - DNA, 7795 bp, Homo sapiens
tgggcggagggaaggcggcaagagctgcggagcccctggaaggtgagaaggactcggagagggaagaaggcccgagactcgagaatgcggggttggggccgggagggatgcaagttccctgggaattagggggtccagcctctgacctccttccggtgaatgttgacgacggctgaattgatcactgattctcaaggggggcatcggacatctgggacccttaagagggcctttgccgatcacacacctgcagccccctgcccgttagaactcctgcactcccccttgccccgtcttacaaatggagaaactgagcccactcccccagatcctaagtcccgcttgatgtaaaggaaagaaccctggcgtaagggtctgggtctgaggtcccagttccggcctggtcacctttagcaacttcctgcccctctgtcagcgtcagattctccatctgtgtcagaggtggaccggcccaaggaaaatagatcaggaatcgctgactccaggagtctctatcccagccccttcgcctgactctttctctggctcccgcggtccctctgagcgattaatgctacataaggtgtgggcagagctggggtcgtgcctccagctgggcaactgcctgtctctctgggtgcctgggtttgctttcttgggcctcggtttccacttctgtagagtggggtgatagtccagcacttcccctgggcgtgtgaaatgtccagcactgccaatattcgttgctgttatcttcggagaacagtgaggggaaaggaatccttgcctgggctgggccaggcaggaggctgggggtcaggacctggaagaggcttccaggtgaggcttggggtggagcctggtgacgaaagcgttaagcccaaactcggtccctggaggattagaggatgatctttaagtccccagctgtcagccctgctcagagcgacagtcctggcagccaatcagatgcgaggacggctgcgggttgcgctcccattggtttactccacccctggggtagcggagcctctttatcgagtgactactgtttgcctcgctctaatcagagcttccaggaaccctgcgctgtgggataaaggaatgaggttcagaaaggggcagggagttgcccgcagccgcaccgcacgtcttcagcccgaccgttgtcctgacctctctgtcccgtcccctgcccagtctcaccatggccttctggacacagctgatgctgctgctctggaagaatttcatgtatcgccggagacagccggtccagctcctggtcgaattgctgtggcctctcttcctcttcttcatcctggtggctgttcgccactcccacccgcccctggagcaccatgaatgccacttcccaaacaagccactgccatcggcgggcaccgtgccctggctccagggtctcatctgtaatgtgaacaacacctgctttccgcagctgacaccgggcgaggagcccgggcgcctgagcaacttcaacgactccctggtctcccggctgctagccgatgcccgcactgtgctgggaggggccagtgcccacaggacgctggctggcctagggaagctgatcgccacgctgagggctgcacgcagcacggcccagcctcaaccaaccaagcagtctccactggaaccacccatgctggatgtcgcggagctgctgacgtcactgctgcgcacggaatccctggggttggcactgggccaagcccaggagcccttgcacagcttgttggaggccgctggggacctggcccaggagctcctggcgctgcgcagcctggtggagcttcgggcactgctgcagagaccccgagggaccagcggccccctggagttgctgtcagaggccctctgcagtgtcaggggacctagcagcacagtgggcccctccctcaactggtacgaggctagtgacctgatggagctggtggggcaggagccagaatccgccctgccagacagcagcctgagccccgcctgctcggagctgattggagccctggacagccacccgctgtcccgcctgctctggagacgcctgaagcctctgatcctcgggaagctactctttgcaccagatacaccttttacccggaagctcatggcccaggtgaaccggaccttcgaggagctcaccctgctgagggatgtccgggaggtgtgggagatgctgggaccccggatcttcaccttcatgaacgacagttccaatgtggccatgctgcagcggctcctgcagatgcaggatgaaggaagaaggcagcccagacctggaggccgggaccacatggaggccctgcgatcctttctggaccctgggagcggtggctacagctggcaggacgcacacgctgatgtggggcacctggtgggcacgctgggccgagtgacggagtgcctgtccttggacaagctggaggcggcaccctcagaggcagccctggtgtcgcgggccctgcaactgctcgcggaacatcgattctgggccggcgtcgtcttcttgggacctgaggactcttcagaccccacagagcacccaaccccagacctgggccccggccacgtgcgcatcaaaatccgcatggacattgacgtggtcacgaggaccaataagatcagggacaggttttgggaccctggcccagccgcggaccccctgaccgacctgcgctacgtgtggggcggcttcgtgtacctgcaagacctggtggagcgtgcagccgtccgcgtgctcagcggcgccaacccccgggccggcctctacctgcagcagatgccctatccgtgctatgtggacgacgtgttcctgcgtgtgctgagccggtcgctgccgctcttcctgacgctggcctggatctactccgtgacactgacagtgaaggccgtggtgcgggagaaggagacgcggctgcgggacaccatgcgcgccatggggctcagccgcgcggtgctctggctaggctggttcctcagctgcctcgggcccttcctgctcagcgccgcactgctggttctggtgctcaagctgggagacatcctcccctacagccacccgggcgtggtcttcctgttcttggcagccttcgcggtggccacggtgacccagagcttcctgctcagcgccttcttctcccgcgccaacctggctgcggcctgcggcggcctggcctacttctccctctacctgccctacgtgctgtgtgtggcttggcgggaccggctgcccgcgggtggccgcgtggccgcgagcctgctgtcgcccgtggccttcggcttcggctgcgagagcctggctctgctggaggagcagggcgagggcgcgcagtggcacaacgtgggcacccggcctacggcagacgtcttcagcctggcccaggtctctggccttctgctgctggacgcggcgctctacggcctcgccacctggtacctggaagctgtgtgcccaggccagtacgggatccctgaaccatggaattttccttttcggaggagctactggtgcggacctcggccccccaagagtccagccccttgccccaccccgctggacccaaaggtgctggtagaagaggcaccgcccggcctgagtcctggcgtctccgttcgcagcctggagaagcgctttcctggaagcccgcagccagccctgcgggggctcagcctggacttctaccagggccacatcaccgccttcctgggccacaacggggccggcaagaccaccaccctgtccatcttgagtggcctcttcccacccagtggtggctctgccttcatcctgggccacgacgtccgctccagcatggccgccatccggccccacctgggcgtctgtcctcagtacaacgtgctgtttgacatgctgaccgtggacgagcacgtctggttctatgggcggctgaagggtctgagtgccgctgtagtgggccccgagcaggaccgtctgctgcaggatgtggggctggtctccaagcagagtgtgcagactcgccacctctctggtgggatgcaacggaagctgtccgtggccattgcctttgtgggcggctcccaagttgttatcctggacgagcctacggctggcgtggatcctgcttcccgccgcggtatttgggagctgctgctcaaataccgagaaggtcgcacgctgatcctctccacccaccacctggatgaggcagagctgctgggagaccgtgtggccgtggtggcaggtggccgcttgtgctgctgtggctccccactcttcctgcgccgtcacctgggctccggctactacctgacgctggtgaaggcccgcctgcccctgaccaccaatgagaaggctgacactgacatggagggcagtgtggacaccaggcaggaaaagaagaatggcagccagggcagcagagtcggcactcctcagctgctggccctggtacagcactgggtgcccggggcacggctggtggaggagctgccacacgagctggtgctggtgctgccctacacgggtgcccatgacggcagcttcgccacactcttccgagagctagacacgcggctggcggagctgaggctcactggctacgggatctccgacaccagcctcgaggagatcttcctgaaggtggtggaggagtgtgctgcggacacagatatggaggatggcagctgcgggcagcacctatgcacaggcattgctggcctagacgtaaccctacggctcaagatgccgccacaggagacagcgctggagaacggggaaccagctgggtcagccccagagactgaccagggctctgggccagacgccgtgggccgggtacagggctgggcactgacccgccagcagctccaggccctgcttctcaagcgctttctgcttgcccgccgcagccgccgcggcctgttcgcccagatcgtgctgcctgccctctttgtgggcctggccctcgtgttcagcctcatcgtgcctcctttcgggcactacccggctctgcggctcagtcccaccatgtacggtgctcaggtgtccttcttcagtgaggacgccccaggggaccctggacgtgcccggctgctcgaggcgctgctgcaggaggcaggactggaggagcccccagtgcagcatagctcccacaggttctcggcaccagaagttcctgctgaagtggccaaggtcttggccagtggcaactggaccccagagtctccatccccagcctgccagtgtagccggcccggtgcccggcgcctgctgcccgactgcccggctgcagctggtggtccccctccgccccaggcagtgaccggctctggggaagtggttcagaacctgacaggccggaacctgtctgacttcctggtcaagacctacccgcgcctggtgcgccagggcctgaagactaagaagtgggtgaatgaggtcagatacggaggcttctcgctggggggccgagacccaggcctgccctcgggccaagagttgggccgctcagtggaggagttgtgggcgctgctgagtcccctgcctggcggggccctcgaccgtgtcctgaaaaacctcacagcctgggctcacagcctggatgctcaggacagtctcaagatctggttcaacaacaaaggctggcactccatggtggcctttgtcaaccgagccagcaacgcaatcctccgtgctcacctgcccccaggcccggcccgccacgcccacagcatcaccacactcaaccaccccttgaacctcaccaaggagcagctgtctgaggctgcactgatggcctcctcggtggacgtcctcgtctccatctgtgtggtctttgccatgtcctttgtcccggccagcttcactcttgtcctcattgaggagcgagtcacccgagccaagcacctgcagctcatggggggcctgtcccccaccctctactggcttggcaactttctctgggacatgtgtaactacttggtgccagcatgcatcgtggtgctcatctttctggccttccagcagagggcatatgtggcccctgccaacctgcctgctctcctgctgttgctactactgtatggctggtcgatcacaccgctcatgtacccagcctccttcttcttctccgtgcccagcacagcctatgtggtgctcacctgcataaacctctttattggcatcaatggaagcatggccacctttgtgcttgagctcttctctgatcagaagctgcaggaggtgagccggatcttgaaacaggtcttccttatcttcccccacttctgcttgggccgggggctcattgacatggtgcggaaccaggccatggctgatgcctttgagcgcttgggagacaggcagttccagtcacccctgcgctgggaggtggtcggcaagaacctcttggccatggtgatacaggggcccctcttccttctcttcacactactgctgcagcaccgaagccaactcctgccacagcccagggtgaggtctctgccactcctgggagaggaggacgaggatgtagcccgtgaacgggagcgggtggtccaaggagccacccagggggatgtgttggtgctgaggaacttgaccaaggtataccgtgggcagaggatgccagctgttgaccgcttgtgcctggggattccccctggtgagtgttttgggctgctgggtgtgaatggagcagggaagacgtccacgtttcgcatggtgacgggggacacattggccagcaggggcgaggctgtgctggcaggccacagcgtggcccgggaacccagtgctgcgcacctcagcatgggatactgccctcaatccgatgccatctttgagctgctgacgggccgcgagcacctggagctgcttgcgcgcctgcgcggtgtcccggaggcccaggttgcccagaccgctggctcgggcctggcgcgtctgggactctcatggtacgcagaccggcctgcaggcacctacagcggagggaacaaacgcaagctggcgacggccctggcgctggttggggacccagccgtggtgtttctggacgagccgaccacaggcatggaccccagcgcgcggcgcttcctttggaacagccttttggccgtggtgcgggagggccgttcagtgatgctcacctcccatagcatggaggagtgtgaagcgctctgctcgcgcctagccatcatggtgaatgggcggttccgctgcctgggcagcccgcaacatctcaagggcagattcgcggcgggtcacacactgaccctgcgggtgcccgccgcaaggtcccagccggcagcggccttcgtggcggccgagttccctgggtcggagctgcgcgaggcacatggaggccgcctgcgcttccagctgccgccgggagggcgctgcgccctggcgcgcgtctttggagagctggcggtgcacggcgcagagcacggcgtggaggacttttccgtgagccagacgatgctggaggaggtattcttgtacttctccaaggaccaggggaaggacgaggacaccgaagagcagaaggaggcaggagtgggagtggaccccgcgccaggcctgcagcaccccaaacgcgtcagccagttcctcgatgaccctagcactgccgagactgtgctctgagcctccctcccctgcggggccgcggggaggccctgggaatggcaagggcaaggtagagtgcctaggagccctggactcaggctggcagaggggctggtgccctggagaaaataaagagaaggctggagagaagccgtggtggtgaaa

SEQ ID NO: 9 - DNA, 20 bp, Artificial Sequence
  Description of Artificial Sequence Primer
agccagcaacgcaatcctcc

SEQ ID NO: 10 - DNA, 20 bp, Artificial Sequence
  Description of Artificial Sequence Primer
cgcaccatgtcaatgagccc

SEQ ID NO: 11 - DNA, 22 bp, Artificial Sequence
  Description of Artificial Sequence Primer
gcggaaagcaggtgttgttcac

SEQ ID NO: 12 - DNA, 21 bp, Artificial Sequence
  Description of Artificial Sequence Primer
cgatggcagtggcttgtttgg